(19) United States
(12) Patent Application Publication
(10) Pub. No.: US 2003/0190642 A1
(43) Pub. Date: Oct. 9, 2003

(54) Thymus expressed human cytochrome p450(p450tec)

(76) Inventors: Glenville Jones, Kingston (CA); Martin P. Petkovich, Kingston (CA); Jay A. White, Kingston Ontario (CA); Heather A. Ramshaw, Napanee Ontario (CA); Wayne A. Stangle, Ottawa (CA)

Correspondence Address: BERESKIN AND PARR, SCOTIA PLAZA, 40 KING STREET WEST-SUITE 4000 BOX 401, TORONTO, ON, M5H 3Y2, CA

(21) Appl. No.: 10/257,378

(22) PCT Filed: Apr. 20, 2001
(86) PCT No.: PCT/CA01/00547

(51) Int. Cl. C12Q 1/68; C07H 21/04; C12N 9/02; C12P 21/02; C12N 5/06

(52) U.S. Cl. 435/6; 435/69.1; 435/189; 435/320.1; 435/325; 536/23.2

(57) ABSTRACT

The present invention relates to a novel human gene encoding a polypeptide that is a member of the cytochrome P450 family. More specifically, the present invention relates to a polynucleotide encoding a novel human polypeptide named cytochrome P450TEC (Thymus Expressed Cytochrome). This invention also relates to P450TEC polypeptides, as well as vectors, host cells, and antibodies directed to P450TEC polypeptides, and the recombinant methods for producing the same. Also provided are therapeutic methods for treating P450TEC-related disorders. The invention further relates to screening methods for identifying agonists and antagonists of P450TEC activity.

### FIELD OF THE INVENTION

The present invention relates to a novel human gene and to the polypeptide encoded thereby which is a member of the cytochrome P450 family. The invention also relates to fragments of the gene and polypeptides and to methods and uses therefor. More specifically, the present invention relates to a polynucleotide encoding a novel human polypeptide named cytochrome P450TEC (Thymus Expressed Cytochrome). This invention also relates to P450TEC polypeptides, as well as vectors, host cells, and antibodies directed to P450TEC polypeptides, and the recombinant methods for producing the same. Also provided are therapeutic methods for treating P450TEC-related disorders and to diagnostic methods of such disorders. The invention further relates to screening methods for identifying modulators, such as agonists and antagonists, of P450TEC activity.

### BACKGROUND OF THE INVENTION

The cytochromes P450 comprise a large gene superfamily that encodes over 500 distinct heme-thiolate proteins that catalyze the oxidation of drugs and numerous other compounds in the body (Nelson et al., Pharmacogenetics 6:1-42 (1996); Guengerich, J. Biol. Chem. 266:10019-10022 (1991)). Since there are at least 500 different cytochrome P450 enzymes, it is of considerable interest in the pharmaceutical and other fields to identify which of these enzymes are most important in the metabolism of individual compounds. There are now numerous examples of adverse drug-drug interactions and side effects that can now be understood in terms of the cytochrome P450 enzymes.

P450 proteins are ubiquitous in living organisms, and have been identified in bacteria, yeast, plants and animals (Nelson et al., Pharmacogenetics 6:1-42 (1996); and Nelson, Arch. Biochem. Biophys. 369:1-10 (1999)). The P450 enzymes catalyze the metabolism of a wide variety of drugs, xenobiotics, carcinogens, mutagens, and pesticides, and are responsible for the bioactivation of numerous endogenous compounds including steroids, prostaglandins, bile acids and fatty acids (Nelson et al., Pharmacogenetics 6:1-42 (1996); Guengerich, J. Biol. Chem. 266:10019-10022 (1991); and Nebert et al., DNA 8:1-13 (1989)).

Cytochrome P450 metabolism of xenobiotics can result in detoxification of toxic compounds by their conjugation into excretable forms or can result in activation of compounds into metabolites that are toxic, mutagenic, or carcinogenic. Many steroids are deactivated by cytochrome P450-catalyzed oxidation.

In 1979, Roberts et al. (J. Biol. Chem. 254:6296-6302 (1979)) first postulated that the catabolism of retinoic acid (RA) was mediated by a P450 enzyme. Several P450s have since been shown to metabolize RA, including P450 proteins from human, zebrafish and mouse. For example, human P450RAI, which is induced by RA, metabolizes RA to more polar derivatives including 4-hydroxy retinoic acid (4-OH-RA) and 4-oxo retinoic acid (4-oxo RA) (White et al., J. Biol. Chem. 271:29922 29927 (1996)).

Active forms of RA, which are derived from vitamin A, are involved in regulating gene expression during development, in regeneration, and in the growth and differentiation of epithelial tissues, and have anticarcinogenic and antiturnoral properties. Since RA is useful as an antitumor agent, it is desirable to maintain high tissue levels of RA. Thus, cytochrome P450 inhibitors that block RA metabolism, resulting in increased tissue levels of RA, may be useful therapeutic agents in the treatment of cancers, such as prostate cancer (Wouters et al., Cancer Res. 52:2841-2846 (1992); and De Coster et al., J. Ster. Biochem. Mol. Biol. 56:133-143 (1996)).

The expression of P450RAI appears to be dependent on the continuous presence of RA in some tissues and cells. Thus, P450RAI regulation by RA appears to form an autoregulatory feedback loop that functions to limit the local concentrations of RA. Essentially, when normal physiological levels of RA are exceeded, P450RAI induction normalizes RA levels (Butler and Fontana, Cancer Res. 52:6164-6167 (1992);Wouter et al., Cancer Res. 52:2841-2846 (1992); and White et al., J. Biol. Chem. 272:18538-18541 (1997)).

Cytochrome P450 proteins are not only involved in the catabolism of RA, but have also been shown to play a role in the metabolism of fatty acids. In particular, P450 inhibitors were observed to block arachidonic acid-induced platelet aggregation and the formation of aggregation factors from arachidonic acid by platelet microsomal enzymes (Cinti and Feinstein, Biochem. Biophys. Res. Commun. 73:171-179 (1976)).

Two cytochrome P450s from the teleost Fundulus heteroclitus, CYP2N1 and CYP2N2, have been shown to function as active arachidonic acid epoxygenases and hydroxylases (Oleksiak et al., J. Biol. Chem. 275:2312-2321 (2000)). Arachidonic acid is a long chain polyunsaturated fatty acid (PUFA) of the omega-6 class (5, 8, 11, 14-eicosatetraenoic acid, i.e., C20:4). Arachidonic acid is the most abundant C20 PUFA in the human body. It is particularly prevalent in organ, muscle and blood tissues, serving a major role as a structural lipid associated predominantly with phospholipids in blood, liver, muscle and other major organ systems.

In addition to its primary role as a structural lipid, arachidonic acid is the direct precursor for a number of circulating eicosanoids: the thromboxanes, leukotrienes, and prostaglandins. Thus, cytochrome P450 epoxygenases and hydroxylases play an important role in the conversion of arachidonic acid to eicosanoids.

The various prostaglandins are grouped into several categories (A-I), which are distinguished by varying substituents on the five-carbon ring introduced into the twenty-carbon fatty acid precursor during prostaglandin synthesis. These groups can be further subdivided based upon the number, and position, of double bonds in the prostaglandins' carbon chains. Examples of prostaglandins are prostaglandin E2(PGE2), prostacyclin I2 (PG2), thromboxane A2 (TxA2), and leukotrienes B4 (LTB4) and C4 (LTC4). (For a review, see, e.g., Goodman and Gilman's The Pharmacological Basis of Therapeutics, Goodman and Gilman, eds., Pergamon Press, New York, pp. 600-611 (1990); and Stryer, Biochemistry (3rd edition), W. H. Freeman and Co., New York, pp. 991-992 (1988).)

Eicosanoids have a broad spectrum of biological activities including regulatory effects on lipoprotein metabolism, blood theology, vascular tone, leucocyte function and platelet activation. For example, E series prostaglandins can affect smooth vascular muscle, e.g., arterioles, precapillaries, sphincters and postcapillary venules, and can be potent vasodilators. Prostaglandins, and related derivatives, can affect the functioning of blood cells, particularly neutrophils and platelets. Uterine contractions can also be affected by PGE, PGF and PGI action. Prostaglandins can also affect renal physiology and central nervous system and afferent nerve function. Various endocrine tissues typically respond to prostaglandins. Furthermore, prostaglandins can modulate inflammatory responses and ameliorate toxemic disorders. Prostaglandins are believed to act on their target cells by way of cellular surface receptors that are thought to be coupled to second messenger systems by which prostaglandin action is mediated.

It is clear that cytochrome P450 epoxygenases and hydroxylases play an important role in the conversion of arachidonic acid to eicosanoids. However, relatively few vertebrate P450s have been identified. Thus, the search continues to exist for P450 polypeptides that play a role in arachidonic acid catalysis.

### SUMMARY OF THE INVENTION

The present invention relates to a novel polynucleotide and the encoded polypeptide of P450TEC. Moreover, the present invention relates to vectors, host cells, antibodies, and recombinant methods for producing P450TEC polypeptides and polynucleotides. Also provided are diagnostic methods for detecting disorders related to the P450TEC genes and polypeptides, and therapeutic methods for treating such disorders. The invention also relates to screening methods for identifying binding partners of P450TEC. The invention further relates to screening methods for identifying agonists and antagonists of P450TEC activity.

In one embodiment, the present inventors have cloned and characterized for the first time human P450TEC. In one embodiment the P450TEC metabolizes arachidonic acid. In another embodiment, P450TEC is a human hydroxylase, preferably a microsomal hydroxylase. In another embodiment the P450TEC is isolated from thymus, cerebral or renal tissue, most preferably from the thymus. These findings have important implications in terms of increased understanding of cytochrome P450TEC activity, and the arachidonic acid metabolic pathway and application to associated disease states.

Accordingly, the present invention provides an isolated polynucleotide comprising a nucleotide sequence encoding a P450TEC, preferably a human P450TEC and to variants, homologs, analogs thereof and to fragments thereof. Complimentary polynucleotide sequences to the polynucleotides of the invention are also encompassed within the scope of the invention.

In a preferred embodiment, an isolated nucleic acid molecule is provided having a nucleic acid sequence as shown in SEQ ID No 16. Most preferably, the nucleic acid molcule is purified and isolated. In one embodiment the nucleic acid mocule or polynucleotide of the invention comprises: (a) a nucleic acid sequence as shown in SEQ ID NO 16 wherein T can also be U; (b) nucleic acid sequences complementary to (a); (c) nucleic acid sequences which are homologous to (a) or (b); or, (d) a fragment of (a) to (c) that is at least 10, preferably at least 15 bases, most preferably 20 to 30 bases, and which will hybridize to (a) to (c) under stringent hybridization conditions. In a further embodiment, the invention provides polynucleotides that consist of the isolated polynucleotides noted herein. In another embodiment, the invention provides nucleic acid molecules that are variants, homologs, analogs or fragments of those noted above.

In another embodiment the invention provides an isolated nucleic acid molecule comprising a polynucleotide having a sequence selected from the group consisting of:

(i) a polynucleotide encoding a polypeptide comprising amino acids from about 1 to 544 of SEQ ID NO.17;

(ii) a polynucleotide consisting of the nucleotide sequence of SEQ ID NO. 16;

(iii) a polynucleotide consisting of the nucleotide sequence of residues 40-1672 of SEQ ID No.16;

(iv) a polynucleotide encoding the amino acid sequence encoded by the cDNA clone contained in ATCC Deposit No. PTA-1785;

(v) a polynucleotide fragment of SEQ ID No. 16 or a polynucleotide fragment of the cDNA sequence contained in ATCC Deposit No. PTA-1785;

(vi) a polunucleotide encoding a polypeptide domain of SEQ ID NO. 17 or the cDNA sequence contained in ATCC Deposit No. PTA-1785;

(vii) a polynucleotide encoding a polypetpide epitope of SEQ ID NO. 17 or the cDNA sequence contained in ATCC Deposit No. PTA-1785;

(viii) a polynucleotide encoding a polypeptide of SEQ ID NO. 17 or the cDNA sequence included in ATCC Deposit No. PTA-1785 having biological activity;

(ix) a polynucleotide that differs from any of the nucleic acid molceules of (i) to (viii) due to the degeneracy of the genetic code;

(x) a polynucleotide that is a variant of SEQ ID. NO. 16;

(xi) a polynucleotide that is an allelic variant of SEQ ID NO.16;

(xii) a polynucleotide that encodes a species homologue of SEQ ID NO. 16;

(xiii) a polynucleotide that is complimentary to any of the polynucleotides of (i) to (xii); and

(xiv) a polynucleotide of (i)-(xiii) wherein T can also be U.

The present invention also includes the P450TEC polypeptide. In one embodiment, the invention provides a polypeptide having an amino acid sequence as shown in SEQ ID NO 17 and to variants, homologs, and analogs, insertions, deletions, substitutions, and mutations thereto. The invention also comprises polypeptides comprising fragments of the amino acid sequence of SEQ ID NO 17 or to their respective variants, homologs, analogs, insertions, deletions, substitutions and mutations. In another embodiment the fragments preferably comprise at least 10, most preferably at least 14 amino acid residues and are most preferably antigenic, immunogenic and or are an eptiope of P450TEC. In another embodiment the invention provides polypeptides encoded by a polynucleotide having the sequence of SEQ ID NO 16, or to variants, homologs, analogs or fragments thereof.

In yet another embodiment the invention provides an isolated polypeptide comprising an amino acid sequence selected from the group consisting of:

(a) a polypeptide fragment of SEQ ID NO:17 or the encoded sequence included in ATCC Deposit No. PTA-1785;

(b) a polypeptide fragment of SEQ ID NO:17 or the encoded sequence included in ATCC Deposit No. PTA-1785 having biological activity;

(c) a polypeptide domain of SEQ ID NO:17 or the encoded sequence included in ATCC Deposit No. PTA-1785;

(d) a polypeptide epitope of SEQ ID NO:17 or the encoded sequence included in ATCC Deposit No. PTA-1785;

(e) a mature form of a secreted protein;

(f) a full length secreted protein;

(g) a variant of SEQ ID NO:17;

(h) an allelic variant of SEQ ID NO:17; or

(i) a species homologue of SEQ ID NO:17.

Accordingly, in one embodiment the invention relates to vectors, host cells comprising the polynucleotides of the invention or that can express the polypeptides of the invention. Antibodies to the polypetides of the invention are also encompassed within the scope of this invention. The invention further provides recombinant methods for producing P450TEC polypeptides and polynucleotides of the invention. In one embodiment, the invention provides a polynucleotide of the invention operationally linked to an expression control sequence in a suitable expression vector. In another embodiment, the expression vector comprising a polynucleotide of the invention is capable of being activated to express the peptide which is encoded by the polynucleotide and is capable of being transformed or transfected into a suitable host cell. Such transformed or transfected cells are also encompassed with the scope of this invention.

The invention also provides a method of preparing a polypeptide of the invention utilizing a polynucleotide of the invention. In one embodiment, a method for preparing the polypeptide, preferably P450TEC, comprising: transforming a host cell with a recombinant expression vector comprising a polynucleotide of the invention; (b) selecting transformed host cells from untransformed host cells; (c) culturing a selected transformed host cell under conditions which allow expression of the protein; and (d) isolating the protein.

In yet another embodiment, the invention also includes diagnostic methods for detecting and screening for disorders related to P450TEC gene expression and polypeptides and to therapeutic methods for treating such disorders.

As such, the invention also includes a method for detecting a disease associated with P450TEC expression in an animal. “A disease associated with P450TEC expression” as used herein means any disease or condition which can be affected or characterized by the level of P450TEC expression or activity. This includes, without limitation, diseases affected by, high, normal, reduced or non-existent expression of P450TEC or expression of mutated P450TEC. A disease associated with P450TEC expression includes but is not limited to diseases associated arachidonic acid metabolism. The method comprises assaying for P450TEC from a sample, such as a biopsy, or other cellular or tissue sample, from an animal susceptible of having such a disease. In one embodiment, the method comprises contacting the sample with an antibody of the invention which binds P450TEC, and measuring the amount of antibody bound to P450TEC in the sample, or unreacted antibody. In another embodiment, the method involves detecting the presence of a nucleic acid molecule having a sequence encoding aP450TEC, comprising contacting the sample with a nucleotide probe which hybridizes with the nucleic acid molecule, preferably mRNA or cDNA to form a hybridization product under conditions which permit the formation of the hybridization product, and assaying for the hybridization product.

The invention further includes a kit for detecting a disease associated with P450TEC expression and/or activity in a sample comprising an antibody of the invention, preferably a monoclonal antibody. Preferably directions for its use is also provided. The kit may also contain reagents which are required for binding of the antibody to a P450TEC protein in the sample.

The invention also provides a kit for detecting the presence of a polynucleotide having a sequence encoding a polypeptide of, related to or analogous to a polypeptide of the invention, comprising a nucleotide probe which hybridizes with the nucleic acid molecule, reagents required for hybridization of the nucleotide probe with the nucleic acid molecule, and directions for its use. In another embodiment the kit comrpises an antibody to P450TEC, that can be labelled or otherwise detected when bound to P450TEC.

The invention also includes screening methods for identifying binding partners of P450TEC. In addition, the invention relates to screening methods for identifying modulators, such as agonists and antagonists, of P450TEC activity. In one embodiment such modulators of P450TEC activity or expression can include antibodies to P450TEC and antisense polynucleotides to the P450TEC gene or fragment thereof.

The invention further provides a method of treating or preventing a disease associated with P450TEC expression or activity comprising administering an effective amount of an agent that activates, simulates or inhibits P450 expression and/or activity, as the situation requires, to an animal in need thereof. In a preferred embodiment, P450TEC, a therapeutically active fragment thereof, or an agent which activates or simulates P450TEC expression is administered to the animal in need thereof to treat a disease or condition associated with P450TEC deficiency or that could benefit from P450TEC expression or activity. In another embodiment the disease is associated with expression of P450TEC that needs to be suppressed or inhibited and the method of treatment comprises administration of an effective amount of an agent that inhibits P450TEC expression and/or activity such as an antibody to P450TEC, a mutation thereof, or an antisense nucleic acid molecule to all or part of the polynucleotide encoding P450TEC or regulatory sequence thereof.

In another embodiment the invention provides pharmaceutical compositions comprising a modulator of P450TEC activity and a pharmaceutical acceptable carrier. In another embodiment, the pharmaceutical composition of the invention comprises P450TEC(preferably a soluble form thereof) or a therapeutically effective fragment thereof and a pharmaceutically acceptable carrier. In another embodiment the pharmaceutical compositions of the invention comprise both a modulator of P450TEC activity and P450TEC(preferably a soluble form thereof) or a therapeutically effective fragment thereof. In a further embodiment the pharmaceutical compositions of the invention can further comprise any one or more of: (a) arachidonic acid and b) NADPH.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

FIGS. 1A-B. FIG. 1A shows the DNA coding sequence (SEQ ID NO:16) of P450TEC. The ORF starts at a predicted ATG start codon at nucleotide 40 and ends at a stop codon at nucleotide 1672. FIG. 1B shows the deduced amino acid sequence (SEQ ID NO:17) of P450TEC.

FIG. 2 is an alignment of the amino acid sequence of the P450TEC protein (SEQ ID NO:17) and the translation products of the piscine CYP2N1 (SEQ ID NO:53) and CYP2N2 (SEQ ID NO:54) genes, two homologs of P450TEC. Identical amino acids between the three polypeptides are boxed, while conservative amino acids have dots between them. By examining the regions of amino acids with boxes and/or dots between them, the skilled artisan can readily identify conserved domains between the polypeptides.

FIG. 3 shows an analysis of predicted epitope regions within the P450TEC amino acid sequence (SEQ ID NO:17). In the “Parker Antigenicity Profile” graph, the positive peaks indicate predicted locations of the highly antigenic regions of the P450TEC protein, i.e., regions from which epitope-bearing peptides of the invention can be obtained. The Parker method (Parker et al, Biochem. 25:5425 5432 (1986)) predicts the location of antigenetic determinants by finding the area of greatest local hydrophobicity. Peaks 1-12 approximately correspond to residues: M 1-R19, R54-P67, S85-I101, S135-V159, K163-G191, K198-I224, I231-F254, F284-L342, N371-Q411, H423-P437, R45 I -Q494, and A512-F528, respectively. The regions defined by this graph are contemplated by the present invention.

FIG. 4 shows an analysis of the functional domains and cytochrome benchmarks of the P450TEC amino acid sequence (SEQ ID NO:17). The domains and benchmarks numbered 1-14 approximately encompass amino acid residues M1 to 159, P60 to F72, W86 to R90, V113 to G115, S157 to V159, A352 to T356, P372, G387, E409 to R412, Y434 to G439, W456 to F467, F483 to G492, M508 to F511 and R543-R544, respectively. These domains and benchmarks constitute the predicted functional domains of the P450TEC protein and are contemplated by the present invention. The reference for benchmark predictions can be found in Cytochrome P450 nomenclature (Nelson, D. R., Methods in Molecular Biology, Vol. 107: Cytochrome P450 Protocols, Cytochrome P450 Nomenclature, pp. 15-24, Phillips, I. R. and Shephard, E. A., eds., Humana Press Inc., Totowa, N.J. (1998)).

FIG. 5 (Panels A-C) shows a multiple tissue Northern dot blot analysis of mRNAs obtained from 76 different human tissues or cell lines. Panel A shows that P450TEC is highly expressed in adult and fetal thymus.

The tissue expression of P450TEC was detected using an α-[³²P]-dATP labeled probe consisting of nucleotides 1400 to 1779 of SEQ ID NO:16. The filter was hybridized overnight, washed with 0.1×SSC, 0.5% SDS and exposed to X-ray film for 24 hours. Panel B: The same blot was re-hybridized with an α-[³²P]-dATP labeled human ubiquitin probe control as supplied by the manufacturer, illustrating the similarities in RNA loading. Panel C shows the location of the various human tissue mRNAs on the blot used in Panels A and B.

FIG. 6 shows a multiple tissue Northern blot analysis of mRNAs obtained from 12 different human tissues. Upper panel: P450TEC is predominantly expressed in thymus, heart and kidney. The blot was probed with α[³²P]-dATP labeled probe consisting of nucleotides 1400 to 1779 of SEQ ID NO:16. The blot was hybridized for 2 hours, washed under low stringency conditions with 2×SSC, 0.5% SDS and exposed to X-ray film for I week. Transcripts according to P450TEC were observed between 4.4 Kb and 7.5 Kb. Bottom panel: The same blot was re-hybridized with an α[32P]- dATP labeled human β-actin probe control as supplied by the manufacturer to control for the amount of mRNA loaded.

FIG. 7 are representative CO-reduced difference spectra of microsomes. After addition of 1-2 mg of sodium dithionite, microsomes were saturated with CO and spectrum was measured between 400-500 nm. FIG. 7A is a CO-reduced difference spectrum of P450TEC expressed in baculovirus-Sf9 insect cell microsomes, FIG. 7B is a CO-reduce difference spectrum of uninfected Sf9 insect cell microsomes.

FIG. 8 are representative HPLC (reversed-phase) elution profiles showing [1-14C] arachidonic acid metabolism by baculovirus-Sf9-expressed P450TEC microsomes. FIG. 8A is the profile of P450TEC microsomes without NADPH-CYP oxidoreductase+b5 and NADPH and in the presence of 20 μM [1-14C] arachidonic acid. FIG. 8B is the elution profile of CYP 2C9 microsomes (Gentest, Woburn, Mass., USA)+NADPH and in the presence of 20 μM [1-14C] arachidonic acid. FIG. 8C is the eltuion profile of P450 TEC microsomes without NADPH-CYP oxidoreductase+b5 in the presence of NADPH and 20 μM [1-14C] arachidonic acid. FIG. 8D is the elution profile of P450TEC microsomes in the presence of NADPH-CYP oxidoreductase+b5, NADPH and 20 μM [1-14C] arachidonic acid. FIG. 8E is the elution profile of P450TEC microsomes in the presence of NADPH-CYP oxidoreductase+b5, NADPH and 1 μM [1-14C] arachidonic acid.

FIG. 9 is a linear graph showing time dependent arachidonic metabolite(s) formation by P450TEC microsomes. Microsomes were incubated with 5 μM [1-14C] arachidonic acid in the presence of NADPH-CYP oxidoreductase+b5 and NADPH.

FIG. 10 is an HPLC (reverse-phase) elution profile showing arachidonic acid metabolism by P450TEC using LC-MS solvent gradient (See text).

FIG. 11 are the LC-MS elution profile showing arachidonic acid metabolism by P450TEC. FIG. 11A is the elution profile of P450TEC microsomes in the presence of NADPH-CYP oxidoreductase+b5, and NADPH and FIG. 11 B is the elution profile of P450TEC microsomes in the presence of NADPH-CYP oxidoreductase+b5, NADPH and arachidonic acid.

FIG. 12 is the MS-spectra of peak eluted at 19.78 min of FIG. 11B showing characteristic of arachidonic acid (m/z 303.4).

FIG. 13 is the MS-spectra of peak eluted at 6.24 min of FIG. 11B showing characteristic of arachidonic acid metabolite (m/z 319.4).

FIG. 14 is the MS/MS spectra of the peak eluted at 6.24 min (MS/MS 319( full scan).

FIG. 15 Western blot analysis illustrating duplicate filters probed with polyclonal antibody recognizing P450TEC-specific peptide (99847) (FIG. 15A) and monoclonal antibody binding to histidine tag (anti-H6) (FIG. 15B) and visualized with HRP-conjugated secondary antibodies and ECL. Positions of protein markers is indicated on the left (size listed in kDa).

### DETAILED DESCRIPTION OF THE PREFERRED EMBODIMENTS

I. Definitions

The following definitions are provided to facilitate understanding of certain terms used throughout this specification.

In the present invention, “isolated” refers to material removed from its original environment (e.g., the natural environment if it is naturally occurring), and thus is altered “by the hand of man” from its natural state. For example, an isolated polynucteotide could be part of a vector or a composition of matter, or could be contained within a cell, and still be “isolated” because that vector, composition of matter, or particular cell is not the original environment of the polynucleotide. However, isolated polynucleotides according to the present invention do not include chromosomes.

In the present invention, a “secreted” P450TEC protein refers to a protein capable of being directed to the ER, secretory vesicles, or the extracellular space as a result of a signal sequence, as well as a P450TEC protein released into the extracellular space without necessarily containing a signal sequence. If the P450TEC secreted protein is released into the extracellular space, the P450TEC secreted protein can undergo extracellular processing to produce a “mature” P450TEC protein. Release into the extracellular space can occur by many mechanisms, including exocytosis and proteolytic cleavage. It would be understood by those skilled in the art that P450TEC can be made into a secretory protein by known genetic engineering techniques, e.g., by adding a suitable signal sequence to the peptide that can preferably be processed into a mature P450TEC.

As used herein, a P450TEC “polynucleotide” refers to a molecule having a nucleic acid sequence contained in SEQ ID NO:16 or the cDNA contained within the clone deposited with the ATCC. For example, the P450TEC polynucleotide can contain the nucleotide sequence of the full length cDNA sequence, including the 5′ and 3′ untranslated sequences, the coding region, with or without the signal sequence, the secreted protein coding region, as well as fragments, epitopes, domains, and variants of the nucleic acid sequence. Moreover, as used herein, a P450TEC “polypeptide” refers to a molecule having the translated amino acid sequence generated from the polynucleotide as broadly defined.

In the present invention, the full length P450TEC cDNA sequence identified as SEQ ID NO:16 was obtained from a human thymus cDNA library. A representative clone containing the sequence for SEQ ID NO:16 was deposited with the American Type Culture Collection (“ATCC”) on Apr. 26, 2000, and was given the ATCC Deposit Number PTA-1785. The ATCC is located at 10801 University Boulevard, Manassas, Va. 20110-2209, USA. The ATCC deposit was made pursuant to the terms of the Budapest Treaty on the international recognition of the deposit of microorganisms for purposes of patent procedure.

A P450TEC “polynucleotide” also refers to isolated polynucleotides which encode the P450TEC polypeptides, and polynucleotides closely related thereto.

A P450TEC “polynucleotide” also refers to isolated polynucleotides which encode the amino acid sequence shown in SEQ ID NO:17, or a biochemically active fragment thereof.

A P450TEC “polynucleotide” also includes those polynucleotides capable of hybridizing, under stringent hybridization conditions, to sequences contained in SEQ ID NO:16, the complement thereof, or the cDNA within the deposited clone. “Stringent hybridization conditions” refers to an overnight incubation at 42° C. in a solution comprising 50% formamide, 5×SSC (750 mM NaCl, 75 mM sodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at about 65° C.

Of course, a polynucleotide which hybridizes only to polyA+sequences (such as any 3′ terminal polyA+′ tract of a cDNA), or to a complementary stretch of T (or U) residues, would not be included in the definition of “polynucleotide,” since such a polynucleotide would hybridize to any nucleic acid molecule containing a poly (A+) stretch or the complement thereof (e.g., practically any double stranded cDNA clone).

The P450TEC polynucleotide can be composed of any polyribonucleotide or polydeoxyribonucleotide, which may be unmodified RNA or DNA or modified RNA or DNA. For example, P450TEC polynucleotides can be composed of single- and double-stranded DNA, DNA that is a mixture of single- and double-stranded regions, single- and double-stranded RNA, and RNA that is a mixture of single- and double-stranded regions, hybrid molecules comprising DNA and RNA that may be single stranded or, more typically, double-stranded or a mixture of single- and double-stranded regions. In addition, the P450TEC polynucleotides can be composed of triple-stranded regions comprising RNA or DNA or both RNA and DNA. P450TEC polynucleotides may also contain one or more modified bases or DNA or RNA backbones modified for stability or for other reasons. “Modified” bases include, for example, tritiated bases and unusual bases such as inosine. A variety of modifications can be made to DNA and RNA; thus, “polynucleotide” embraces chemically, enzymatically, or metabolically modified forms. Modified forms also encompass analogs of the polynucleotide sequence of the invention, wherein the modification does not alter the utility of the sequences decribed herein. In one embodiment the modified seqeunce or analog may have improved properties over unmodified sequence.

P450TEC polypeptides can be composed of amino acids joined to each other by peptide bonds or modified peptide bonds, i.e., peptide isosteres, and may contain amino acids other than the 20 gene encoded amino acids. The P450TEC polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in the P450TEC polypeptide, including the peptide backbone, the amino acid sidechains and the amino or carboxyl termini. It will be appreciated that the same type of modification may be present in the same or varying degrees at several sites in a given P450TEC polypeptide. Also, a given P450TEC polypeptide may contain many types of modifications. P450TEC polypeptides may be branched, for example, as a result of ubiquitination, and they may be cyclic, 30 with or without branching. Cyclic, branched, and branched cyclic P450TEC polypeptides may result from posttranslation natural processes or may be made by synthetic methods. Modifications include acetylation, acylation, ADP ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross linking, cyclization, disulfide bond formation, demethylation, formation of covalent cross-links, reduction of disulfide bonds into free cysteine, formation of pyroglutamate, formylation, gamma carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins—Structure And Molecular Properties, 2nd Ed., T. E. Creighton, W. H. Freeman and Company, New York (1993); Posttranslational Covalent Modification of Proteins, B. C. Johnson, Ed., Academic Press, New York, pp. 1-12 (1983); Seifter et al., Meth Enzymol 182:626-646 (1990); Rattan et al., Ann NY Acad Sci 663:48-62 (1992).)

“SEQ ID NO:16” refers to a P450TEC polynucleotide sequence while “SEQ ID NO:17 ” refers to a P450TEC polypeptide sequence.

A P450TEC polypeptide “having biological activity” refers to polypeptides exhibiting activity similar, but not necessarily identical to, an activity of a P450TEC polypeptide, including mature forms, as measured in a particular biological assay, with or without dose dependency. In the case where dose dependency does exist, it need not be identical to that of the P450TEC polypeptide, but rather substantially similar to the dose-dependence in a given activity as compared to the P450TEC polypeptide (i.e., the candidate polypeptide will exhibit greater activity or not more than about 25 fold less and, preferably, not more than about tenfold less activity, and most preferably,; not more than about three-fold less activity relative to the P450TEC polypeptide.)

II. P450TEC Polynucleotides and Polypeptides

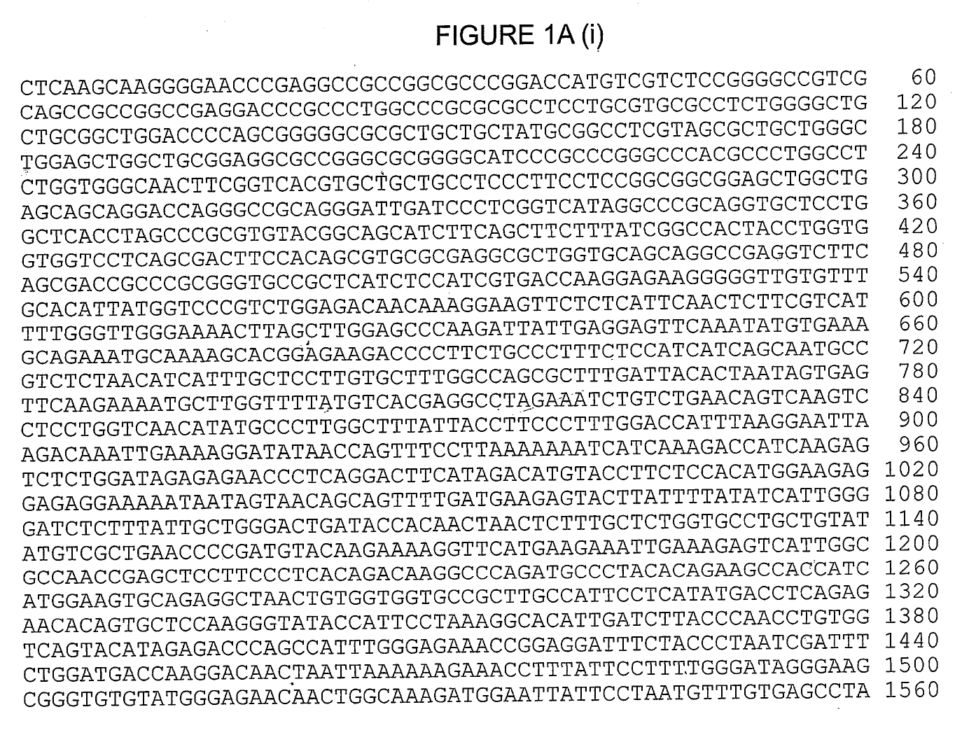

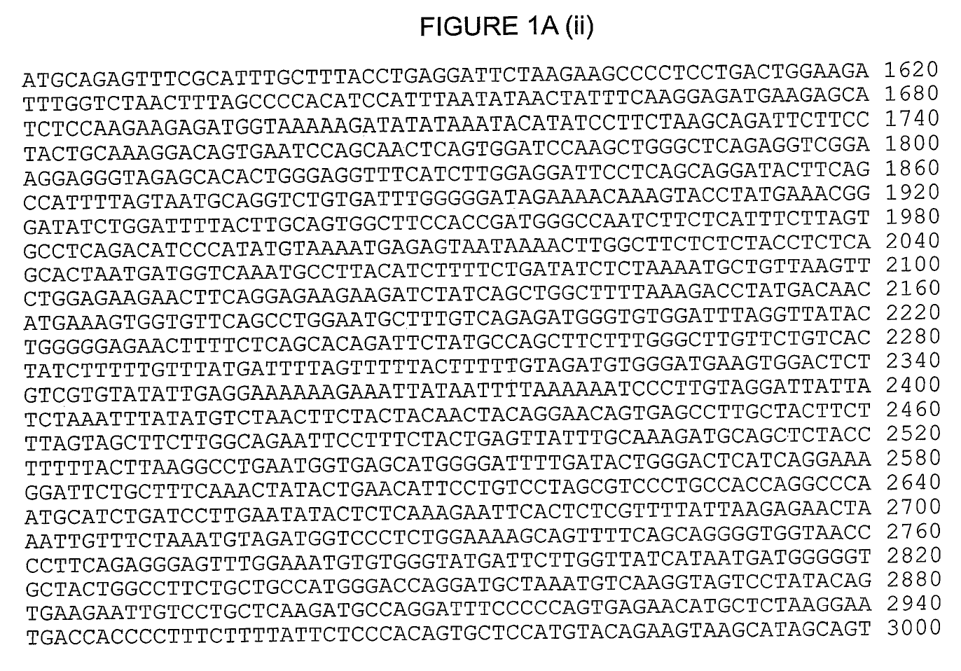

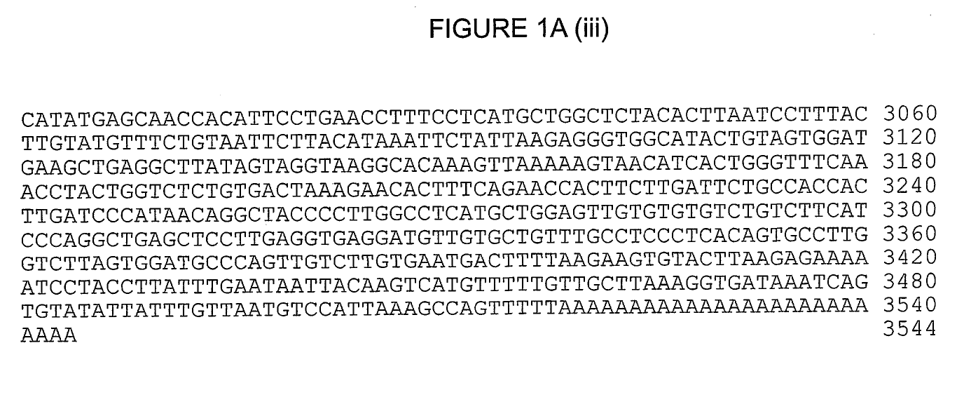

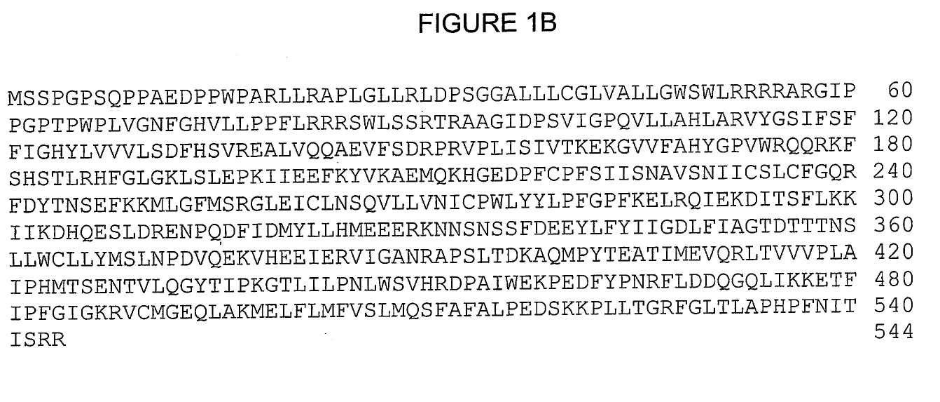

Clone 46c1 was isolated from a human thymus cDNA library. The nucleotide sequence determined by sequencing the 46c1 clone, which is shown in FIG. 1A (SEQ ID NO:16), is 3544 nucleotides in length and contains an open reading frame encoding a polypeptide of 544 amino acid residues. The amino acid sequence of the P450TEC protein is shown in FIG. 1B (SEQ ID NO:17). The open reading frame begins at a N-terminal methionine located at nucleotide position 40, and ends at a stop codon at nucleotide position 1672. Subsequent Northern analysis also showed lower P450TEC expression in heart, kidney, left and right cerebellum, corpus callosum, pituitary and aorta.

The P450TEC gene is located on genomic clone B4P3 (Accession No. AC000016), which has been mapped to chromosome 4 at approximately 4q25.

SEQ ID NO:16 is useful for designing nucleic acid hybridization probes that will detect nucleic acid sequences contained in SEQ ID NO:16 or the cDNA contained in the deposited clone. These probes will also hybridize to nucleic acid molecules in biological samples, thereby enabling a variety of forensic and diagnostic methods of the invention. Similarly, polypeptides identified from SEQ ID NO:17 may be used to generate antibodies which bind specifically to P450TEC.

Nevertheless, DNA sequences generated by sequencing reactions can contain sequencing errors. The errors exist as misidentified nucleotides, or as insertions or deletions of nucleotides in the generated DNA sequence. The erroneously inserted or deleted nucleotides cause frame shifts in the reading frames of the predicted amino acid sequence. In these cases, the predicted amino acid sequence diverges from the actual amino acid sequence, even though the generated DNA sequence may be greater than 99.9% identical to the actual DNA sequence (for example, one base insertion or deletion in an open reading frame of over 1000 bases).

Accordingly, for those applications requiring precision in the nucleotide sequence or the amino acid sequence, the present invention provides not only the generated nucleotide sequence identified as SEQ ID NO:16 and the predicted translated amino acid sequence identified as SEQ ID NO:17, but also a sample of plasmid DNA containing a human cDNA of P450TEC deposited with the ATCC. The nucleotide sequence of the deposited P450TEC clone can readily be determined by sequencing the deposited clone in accordance with known methods. The predicted P450TEC amino acid sequence can then be verified from such deposits. Moreover, the amino acid sequence of the protein encoded by the deposited clone can also be directly determined by peptide sequencing or by expressing the protein in a suitable host cell containing the deposited human P450TEC cDNA, collecting the protein, and determining its sequence.

The present invention also relates to the P450TEC gene corresponding to SEQ ID NO:16 or the deposited clone. The P450TEC gene can be isolated in accordance with known methods using the sequence information disclosed herein. Such methods include preparing probes or primers from the disclosed sequence and identifying or amplifying the P450TEC gene from appropriate sources of genomic material.

Also provided in the present invention are species homologs of P450TEC. Species homologs may be isolated and identified by making suitable probes or primers from the sequences provided herein and screening a suitable nucleic acid source for the desired homologue.

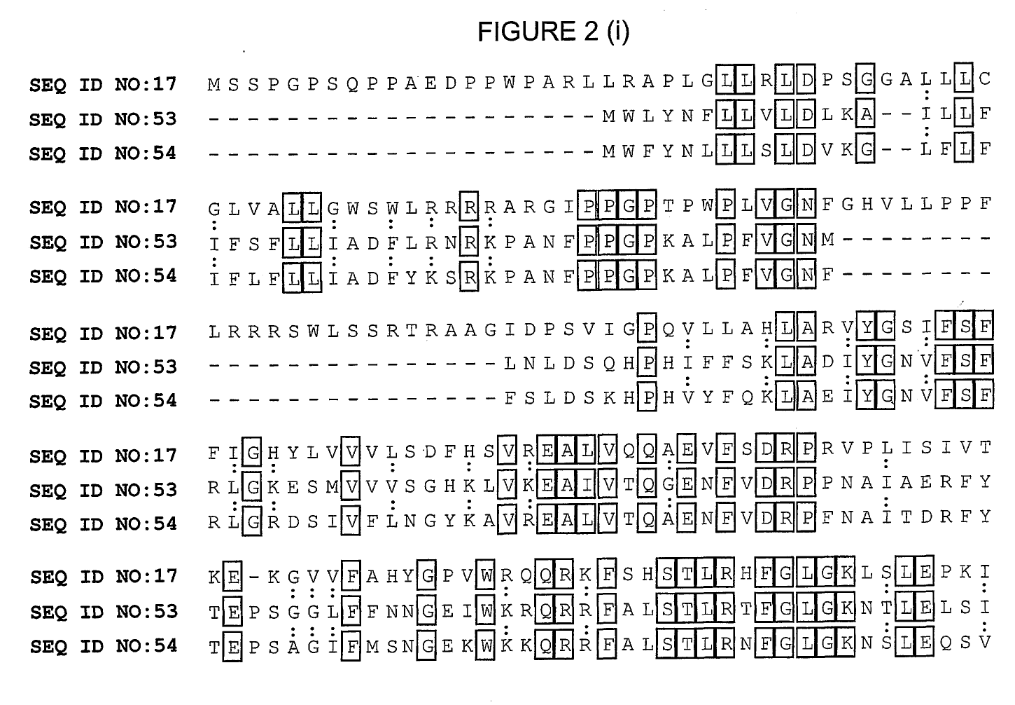

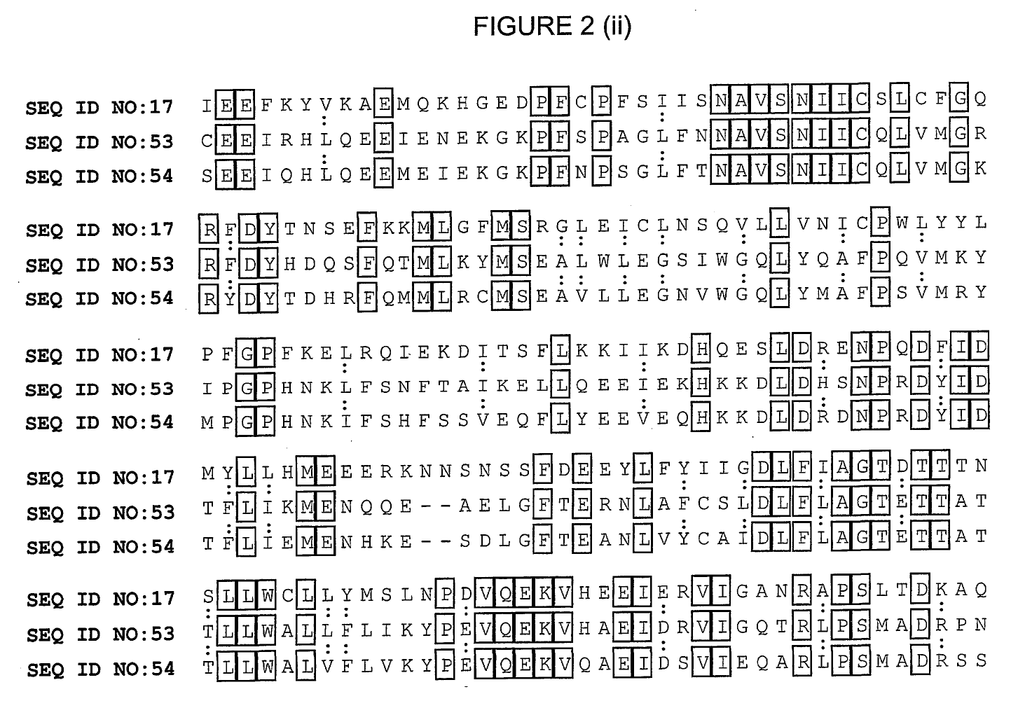

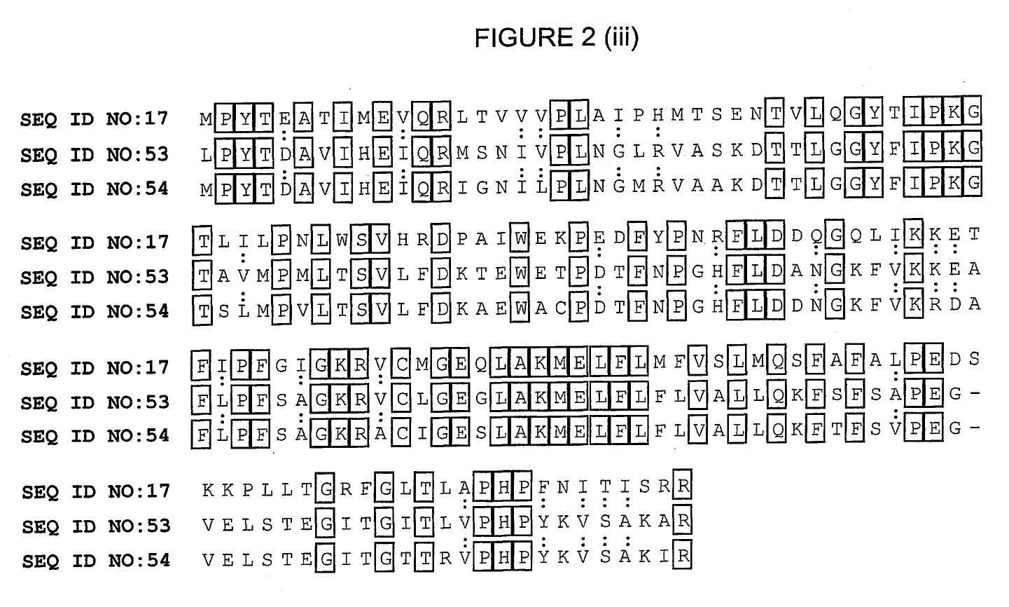

The P450TEC protein shown in FIG. 1B is about 41% identical and 57% similar to two piscine proteins, CYP2N1 (SEQ ID NO:53) and CYP2N2 (SEQ ID NO:54) (see FIG. 2). These proteins have been shown to metabolize arachidonic acid to epoxyeicosatrienoic acids (EETs) (Oleksiak et al., J Biol. Chem. 275:2312-2321 (2000)).

EETs, such as prostaglandins, thromboxanes and leukotrienes, have been shown to activate phosphorylase a, regulate coronary artery, intestine and cerebral vascular tone, modulate Ca2+transport, activate Ca2+-activated K+channels, and modulate the secretion of neuropeptides (Wu et al., J. Biol. Chem. 272:12551-12559 (1997); Kutsky et al., Prostaglandins 26:13-21 (1983); Yoshida et al., Arch. Biochem. Biophys. 353:265-275 (1990); Harder 30 et al., J. Vasc. Res. 32:79-92 (1995); Moffat et al., Am. J. Physiol. 264:H1154 H1160 (1993); Proctor et al., Blood Vessels 26:53-64 (1989); Junier et al., Endocrinol. 126:1534-1540 (1990); Capdevila et al., Endocrinol. 113:421-423 (1983); and Gebremedhin et al., Am. J. Physiol. 263:H519-H525 (1992)).

The EETs have also been shown to affect general physiological processes such as cellular proliferation and tyrosine kinase activity (Harris et al., J. Cell. Physiol. 144:429-437 (1990); Hoebel and Graier, Eur. J. Pharmacol. 346:115-117 (1998)). In addition, prostaglandins are known to stimulate inflammation, regulate blood flow to particular organs, control ion transport across membranes and modulate synaptic transmission.

Thus, it P450TEC may have some activity that is similar to the known activity of the two piscine homologs, namely, the conversion of arachidonic acid to epoxyelcosatrienoic acids.

P450TEC can hydroxylate arachidonic acid. Such metabolites are preferably the monohydroxylated arachidonic acid, such as 5−, 8−, 9−, 11−, 12−, 15−, 19−and 20−HETE, but are not necessarily limited to such. Such metabolites, also have effects relating to calcium, sodium and potassium ion transport and related conditions, vasoconstriction, chemotaxis, platelet aggregation, inflammation, autoimmune disorders. Effects in the immune, cardiovascular and renal systems and in the gastrointestinal tract have also been reported. A person skilled in the art would be familiar with other HETE related effects that have been identified in the art.

The cytochrome P450s are heme-binding proteins that contain the putative family signature, F(XX)G(XXX)C(X)G (X means any residue; conserved residues are in bold). (Nelson, D. R., Methods in Molecular Biology, Vol. 107: Cytochrome P450 Protocols, Cytochrome P450 Nomenclature, pp. 15-24, Phillips, I. R. and Shephard, E. A., eds., Humana Press Inc., Totowa, N.J. (1998)). The heme-binding signature in P450TEC can be found at amino acids 483-492 and contains the motif FGIGKRVCMG (see FIG. 1B and SEQ ID NO:17). P450TEC also contains an oxygen binding domain at amino acids 352-356.

Heme-binding proteins, such as myoglobin, hemoglobin and cytochromes, play an important role in several cellular processes, such as respiration and detoxification. For example, the capacity of myoglobin or hemoglobin to bind oxygen depends on the presence of a heme group. Heme consists of an organic part and an iron atom. The iron atom in heme alternates between a ferrous (+2) and a ferric (+3) state; however, only heme containing an iron atom in the +2 oxidation state binds oxygen. (For a review, see, e.g., Stryer, Biochemistry (3rd edition), W. H. Freeman and Co., New York, pp. 144 and 404-405 (1988).)

Cytochrome P450s play an important role in the detoxification of toxic substances (xenobiotics), such, as phenobarbital, codeine and morphine, by oxidation. It is the ability of P450s to bind heme and oxygen that enables them to function as oxidative enzymes. (For a review, see, e.g., Darnell et al., Molecular Cell Biology (2nd editition), W. H. Freemand and Co., New York, pp.397 and 981 982 (1990).) Thus, peptides of P450TEC containing the heme binding motif or the oxygen-binding domain are also contemplated by the inventors.

By “P450TEC polypeptide(s)” is meant all forms of P450TEC proteins and polypeptides described herein. The P450TEC polypeptides can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art.

The P450TEC polypeptides may be in the form of a membrane bound protein, a secreted protein, including the mature form, or may be a part of a larger protein, such as a fusion protein (see below). It is often advantageous to include an additional amino acid sequence which contains secretory or leader sequences, pro-sequences, sequences which aid in purification, such as multiple histidine residues, or an additional sequence for stability during recombinant production.

P450TEC polypeptides are preferably provided in an isolated form, and preferably are substantially purified. A recombinantly produced version of a P450TEC polypeptide, including the secreted polypeptide, can be substantially purified by the one-step method described in Smith and Johnson, Gene 67:31-40 (1988). P450TEC polypeptides also can be purified from natural or recombinant sources using antibodies of the invention raised against the P450TEC protein in methods which are well known in the art.

Ill. Polynucleotide and Polypeptide Variants

“Variant” refers to a polynucleotide or polypeptide differing from the P450TEC polynucleotide or polypeptide, but retaining essential properties thereof. Generally, variants are overall closely similar, and, in many regions, identical to the P450TEC polynucleotide or polypeptide.

By a polynucleotide having a nucleotide sequence at least, for example, 90% “identical” to a reference nucleotide sequence of the present invention, it is intended that the nucleotide sequence of the polynucleotide is identical to the reference sequence except that the polynucleotide sequence may include up to ten point mutations per each 100 nucleotides of the reference nucleotide sequence encoding the P450TEC polypeptide. In other words, to obtain a polynucleotide having a nucleotide sequence at least 90% identical to a reference nucleotide sequence, up to 10% of the nucleotides in the reference sequence may be deleted or substituted with another nucleotide, or a number of nucleotides up to 10% of the total nucleotides in the reference sequence may be inserted into the reference sequence. The query sequence may be an entire sequence shown of SEQ ID NO:16, the ORF (open reading frame), or any fragment specified as described herein.

As a practical matter, whether any particular nucleic acid molecule or polypeptide is at least 90%, 95%, 96%, 97%, 98% or 99% identical to a nucleotide sequence of the presence invention can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al., Comp. App. Biosci. 6:237-245 (1990). In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identity are: Matrix=Unitary, k-tuple=4, Mismatch Penalty=1, Joining Penalty=30, Randomization Group Length=0, Cutoff Score=1, Gap Penalty=5, Gap Size Penalty 0.05, Window Size=500 or the length of the subject nucleotide sequence, whichever is shorter.

If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score.

For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 unpaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for. No other manual corrections are to be made for the purposes of the present invention.

By a polypeptide having an amino acid sequence at least, for example, 90% “identical” to a query amino acid sequence of the present invention, it is intended that the amino acid sequence of the subject polypeptide is identical to the query sequence except that the subject polypeptide sequence may include up to ten amino acid alterations per each 100 amino acids of the query amino acid sequence. In other words, to obtain a polypeptide having an amino acid sequence at least 90% identical to a query amino acid sequence, up to 10% of the amino acid residues in the subject sequence may be inserted, deleted, or substituted with another amino acid. These alterations of the reference sequence may occur at the amino or carboxyl terminal positions of the reference amino acid sequence or anywhere between those terminal positions, interspersed either individually among residues in the reference sequence or in one or more contiguous groups within the reference sequence.

As a practical matter, whether any particular polypeptide is at least 90%, 95%, 96%, 97%, 98% or 99% identical to, for instance, the amino acid sequences shown in SEQ ID NO:17 or to the amino acid sequence encoded by the deposited DNA clone can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al., Comp. App. Biosci. 6:237-245 (1990). In a sequence alignment the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C terminal residues of the subject sequence.

For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for. No other manual corrections are to be made for the purposes of the present invention. The P450TEC variants may contain alterations in the coding regions, non-coding regions, or both. Especially preferred are polynucleotide variants containing alterations which produce silent substitutions, additions, or deletions, but do not alter the properties or activities of the encoded polypeptide. Nucleotide variants produced by silent substitutions due to the degeneracy of the genetic code are preferred. Moreover, variants in which 5-10, 1-5, or 1-2 amino acids are substituted, deleted, or added in any combination are also preferred. P450TEC polynucleotide variants can be produced for a variety of reasons, e.g., to optimize codon expression for a particular host (change codons in the human mRNA to those preferred by a bacterial host such as E. coli).

Naturally occurring P450TEC variants are called “allelic variants,” and refer to one of several alternate forms of a gene occupying a given locus on a chromosome of an organism (Genes II, Lewin, B., ed., John Wiley & Sons, New York (1985)). These allelic variants can vary at either the polynucleotide and/or polypeptide level. Alternatively, non-naturally occurring variants may be produced by mutagenesis techniques or by direct synthesis.

Using known methods of protein engineering and recombinant DNA technology, variants may be generated to improve or alter the characteristics of the P450TEC polypeptides. For instance, one or more amino acids can be deleted from the N-terminus or C-terminus of the secreted protein without substantial loss of biological function. The authors of Ron et al., J. Biol. Chem. 268: 2984-2988 (1993), reported variant KGF proteins having heparin binding activity even after deleting 3, 8, or 27 amino-terminal amino acid residues. Similarly, Interferon gamma exhibited up to ten times higher activity after deleting 8-10 amino acid residues from the carboxyl terminus of this protein (Dobeli et al., J. Biotechnology 7:199-216 (1988)).

Moreover, ample evidence demonstrates that variants often retain a biological activity similar to that of the naturally occurring protein. For example, Gayle and coworkers J. Biol. Chem 268:22105 22111 (1993) conducted extensive mutational analysis of human cytokine IL-1α. They used random mutagenesis to generate over 3,500 individual IL-1α mutants that averaged 2.5 amino acid changes per variant over the entire length of the molecule. Multiple mutations were examined at every possible amino acid position. The investigators found that “[m]ost of the molecule could be altered with little effect on either [binding or biological activity].” (See, Abstract.) In fact, only 23 unique amino acid sequences, out of more than 3,500 nucleotide sequences examined, produced a protein that significantly differed in activity from wild-type.

Furthermore, even if deleting one or more amino acids from the N terminus or C-terminus of a polypeptide results in modification or loss of one or more biological functions, other biological activities may still be retained. For example, the ability of a deletion variant to induce and/or to bind antibodies which recognize the secreted form will likely be retained when less than the majority of the residues of the secreted form are removed from the N-terminus or C-terminus. Whether a particular polypeptide lacking N- or C terminal residues of a protein retains such immunogenic activities can readily be determined by routine methods described herein and otherwise known in the art.

Thus, the invention further includes P450TEC polypeptide variants which show substantial biological activity. Such variants include deletions, insertions, inversions, repeats, and substitutions selected according to general rules known in the art so as to have little effect on activity. For example, guidance concerning how to make phenotypically silent amino acid substitutions is provided in Bowie, J. U. et al., Science 247:1306-1310 (1990), wherein the authors indicate that there are two main strategies for studying the tolerance of an amino acid sequence to change.

The first strategy exploits the tolerance of amino acid substitutions by natural selection during the process of evolution. By comparing amino acid sequences in different species, conserved amino acids can be identified. These conserved amino acids are likely important for protein function. In contrast, the amino acid positions where substitutions have been tolerated by natural selection indicates that these positions are not critical for protein function. Thus, positions tolerating amino acid substitution could be modified while still maintaining biological activity of the protein.

The second strategy uses genetic engineering to introduce amino acid changes at specific positions of a cloned gene to identify regions critical for protein function. For example, site directed mutagenesis or alanine-scanning mutagenesis (introduction of single alanine mutations at every residue in the molecule) can be used. (Cunningham and Wells, Science 244:1081-1085 (1989).) The resulting mutant molecules can then be tested for biological activity.

As the authors state, these two strategies have revealed that proteins are surprisingly tolerant of amino acid substitutions. The authors further indicate which amino acid changes are likely to be permissive at certain amino acid positions in the protein. For example, most buried (within the tertiary structure of the protein) amino acid residues require nonpolar side chains, whereas few features of surface side chains are generally conserved. Moreover, tolerated conservative amino acid substitutions involve replacement of the aliphatic or hydrophobic amino acids Ala, Val, Leu and lie;

replacement of the hydroxyl residues Ser and Thr; replacement of the acidic residues Asp and Glu; replacement of the amide residues Asn and Gin, replacement of the basic residues Lys, Arg, and His; replacement of the aromatic residues Phe, Tyr, and Trp, and replacement of the small-sized amino acids Ala, Ser, Thr, Met, and Gly.

Besides conservative amino acid substitution, variants of P450TEC include (i) substitutions with one or more of the non-conserved amino acid residues, where the substituted amino acid residues may or may not be one encoded by the genetic code, or (ii) substitution with one or more of amino acid residues having a substituent group, or (iii) fusion of the mature polypeptide with another compound, such as a compound to increase the stability and/or solubility of the polypeptide (for example, polyethylene glycol), or (iv) fusion of the polypeptide with additional amino acids, such as an IgG Fc fusion region peptide, or leader or secretory sequence, or a sequence facilitating purification. Such variant polypeptides are deemed to be within the scope of those skilled in the art from the teachings herein.

For example, P450TEC polypeptide variants containing amino acid substitutions of charged amino acids with other charged or neutral amino acids may produce proteins with improved characteristics, such as less aggregation. Aggregation of pharmaceutical formulations both reduces activity and increases clearance due to the aggregate's immunogenic activity. (Pinckard et al., Clin. Exp. Immunol. 2:331-340 (1967); Robbins et al., Diabetes 36: 838-845 (1987); Cleland et al., Crit. Rev. Therapeutic Drug CarrierSystenis 10:307-377 (1993).)

Although, variants are described in detail with respect polynucleotides and polypeptides having a particular % of identical to a reference seqeunce, it should be noted that polynucleotides and polypeptides that are at least 60% identical to a refereence nucleotide or peptide sequence of the present invention are also intended to be encompassed within the scope of this invention. For instance, polynculeotides and polypeptides that are at least 70%, 75%, 80%, 85% identical to the reference sequence are intended to be encompassed within the scope of the present invention.

IV. Polynucleotide and Polypeptide Fragments

In the present invention, a “polynucleotide fragment” refers to a short polynucleotide having a nucleic acid sequence contained in the deposited clone or shown in SEQ ID NO:16. The short nucleotide fragments are preferably at least about 15 nt, and more preferably at least about 20 nt, still more preferably at least about 30 nt, and even more preferably, at least about 40 nt in length. A fragment “at least 20 nt in length,” for example, is intended to include 20 or more contiguous bases from the CDNA sequence contained in the deposited clone or the nucleotide sequence shown in SEQ ID NO:16. These nucleotide fragments are useful as diagnostic probes and primers as discussed herein. Of course, larger fragments (e.g., 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600 nucleotides) are also useful as diagnostic probes and primers.

Moreover, representative examples of P450TEC polynucleotide fragments include, for example, fragments having a sequence from about nucleotide number 1-50, 51-100, 101-150, 151-200, 201-250, 251-300, 301-350, 351-400, 401-450, 451-500, 501-550, 551-600, 601-650, 651-700, 701-750, 751-800, 801-850, 851-900, 901-950, 951-1000, 1001-1050, 1051-1100, 1151-1200, 1201 1250, 1251-1300, 1301-1350, 1351-1400,1401-1450, 1451-1500, 1501-1550, 1551-1600 or 1601 to the end of SEQ ID NO:16 or the cDNA contained in the deposited clone. In this context “about” includes the particularly recited ranges, larger or smaller by several (5, 4, 3, 2, or 1) nucleotides, at either terminus or at both termini. Preferably, these fragments encode a polypeptide which has biological activity. More preferably, these polynucleotides can be used as probes or primers as discussed herein.

In the present invention, a “polypeptide fragment” refers to a short amino acid sequence contained in SEQ ID NO:17 or encoded by the cDNA contained in the deposited clone. Protein fragments may be “free-standing,” or comprised within a larger polypeptide of which the fragment forms a part or region, most preferably as a single continuous region. Representative examples of polypeptide fragments of the invention, include, for example, fragments from about amino acid number 1-20, 21-40, 41-60, 61-80, 81-100, 101-120, 121-140, 141-160, 161-180, 181-200, 201-220, 221-240, 241 260, 261-280, 281-300, 301-320, 321-340, 341-360, 361-380, 381-400, 401-420, 421-440, 441-460, 461-480, 481-500, 501-520 or 521 to the end of the coding region. Moreover, polypeptide fragments can be about 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 340, 360, 380, 400, 410, 420, 430, 440, 450, 460, 470, or 480 amino acids in length, In this context “about” includes the particularly recited ranges, larger or smaller by several (5, 4, 3, 2, or 1) amino acids, at either extreme or at both extremes.

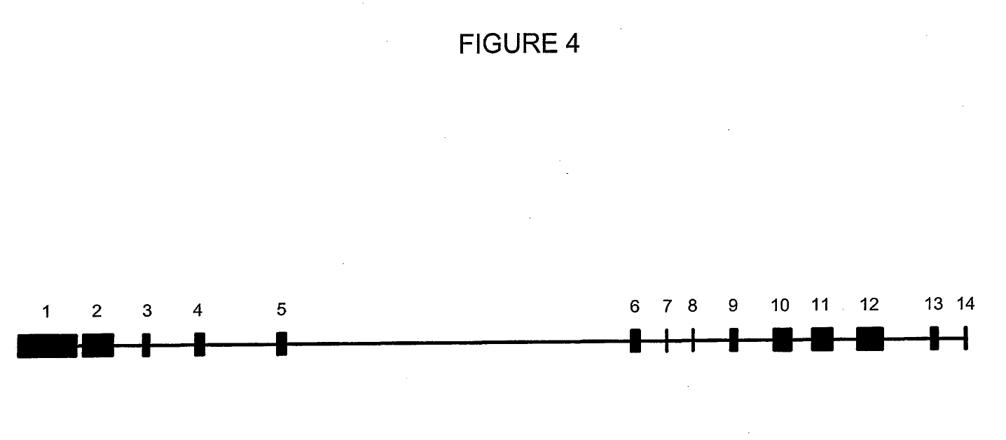

More in particular, the invention provides polynucleotides encoding polypeptide fragments comprising, or alternatively consisting of, the amino acid sequence of residues M1 to I59, P60 to F72, W86 to R90, V113 to G115, S157 to V159, A352 to T356, E409 to R412, Y434 to G439, W456 to F467, F483 to G492, M508 to F511 of the P450TEC sequence shown in SEQ ID NO:17 (see FIG. 4). Polynucleotides encoding these polypeptides are also encompassed by the invention.

Preferred polypeptide fragments include the secreted P450TEC protein as well as the mature form. Further preferred polypeptide fragments include the secreted P450TEC protein or the mature form having a continuous series of deleted residues from the amino or the carboxy terminus, or both. For example, any number of amino acids, ranging from 1-59, can be deleted from the amino terminus of either the secreted P450TEC polypeptide or the mature form. Similarly, any number of amino acids, ranging from 1-33, can be deleted from the carboxy terminus of the secreted P450TEC protein or mature form. Furthermore, any combination of the above amino and carboxy terminus deletions are preferred. Similarly, polynucleotide fragments encoding these P450TEC polypeptide fragments are also preferred.

As mentioned above, even if deletion of one or more amino acids from the N-terminus of a protein results in modification of loss of one or more biological functions of the protein, other biological activities may still be retained. Thus, the ability of shortened P450TEC muteins to induce and/or bind to antibodies which recognize the complete or mature forms of the polypeptides generally will be retained when less than the majority of the residues of the complete or mature polypeptide are removed from the N-terminus. Whether a particular polypeptide lacking N-terminal residues of a complete polypeptide retains such immunologic activities can readily be determined by routine methods described herein and otherwise known in the art. It is not unlikely that a P450TEC mutein with a large number of deleted N-terminal amino acid residues may retain some biological or immunogenic activities. In fact, peptides composed of as few as five P450TEC amino acid residues may often evoke an immune response.

Accordingly, the present invention further provides polypeptides having one or more residues deleted from the amino terminus of the P450TEC amino acid sequence shown in SEQ ID NO:17, up to the lie residue at position number 59 and polynucleotides encoding such polypeptides.

Also as mentioned above, even if deletion of one or more amino acids from the C-terminus of a protein results in modification of loss of one or more biological functions of the protein, other biological activities may still be retained. Thus, the ability of the shortened P450TEC mutein to induce and/or bind to antibodies which recognize the complete or mature forms of the polypeptide generally will be retained when less than the majority of the residues of the complete or mature polypeptide are removed from the C-terminus. Whether a particular polypeptide lacking C terminal residues of a complete polypeptide retains such immunologic activities can readily be determined by routine methods described herein and otherwise known in the art. It is not unlikely that a P450TEC mutein with a large number of deleted C terminal amino acid residues may retain some biological or immunogenic activities.

Accordingly, the present invention further provides polypeptides having one or more residues deleted from the carboxy terminus of the amino acid sequence of the P450TEC polypeptide shown in SEQ ID NO:17, up to the Phe residue at position number 511, and polynucleotides encoding such polypeptides.

The invention also provides polypeptides having one or more amino acids deleted from both the amino and the carboxyl termini of a P450TEC polypeptide, which may be described generally as having residues m-n of SEQ ID NO:17, where m is an integer from 2 to 59 and n is an integer from 511 to 544.

Many polynucleotide sequences, such as EST sequences, are publicly available and accessible through sequence databases. Some of these sequences are related to SEQ ID NO:16 and may have been publicly available prior to conception of the present invention. Preferably, such related polynucleotides are specifically excluded from the scope of the present invention.

For example, the following sequences are related to SEQ ID NO:16, GenBank Accession Nos.: AI216236 (SEQ ID NO:2), AW242436 (SEQ ID NO:18), AI798940 (SEQ ID NO:19), AW274541 (SEQ ID NO:20), AA868720 (SEQ ID NO:21), AW055161 (SEQ ID NO:22), AI952800 (SEQ ID NO:23), AA768918 (SEQ ID NO:24), AI423173 (SEQ ID NO:25), AA988114 (SEQ ID NO:26), AI418926 (SEQ ID NO:27), AI096969 (SEQ ID NO:28), AI359764 (SEQ ID NO:29), AW300154 (SEQ ID NO:30), AA427961 (SEQ ID NO:31), AI023027 (SEQ ID NO:32), AW027423 (SEQ ID NO:33), H15514 (SEQ ID NO:34), AA406316 (SEQ ID NO:35), W19602 (SEQ ID NO:36), AI051004 (SEQ ID NO:37), AA749349 (SEQ ID NO:38), AW183742 (SEQ ID NO:39), AI399966 (SEQ ID NO:40), R67298 (SEQ ID NO:41), AA730070 (SEQ ID NO:42), AW137850 (SEQ ID NO:43), N33755 (SEQ ID NO:44), A1460189 (SEQ ID NO:45), AA418010 (SEQ ID NO:46), C20977 (SEQ ID NO:47), N99120 (SEQ ID NO:48), N44749 (SEQ ID NO:49), AA495783 (SEQ ID NO:50), AA905360 (SEQ ID NO:51) and F12172 (SEQ ID NO:52). Thus, in one embodiment the present invention is directed to polynucleotides comprising one or more of the polynucleotide fragments described herein exclusive of the above-recited ESTs.

In another embodiment, the present invention is also directed to polynucleotides comprising the polynucleotide fragments described herein exclusive of AF090434 (SEQ ID NO:53) and AF090435 (SEQ ID NO:54).

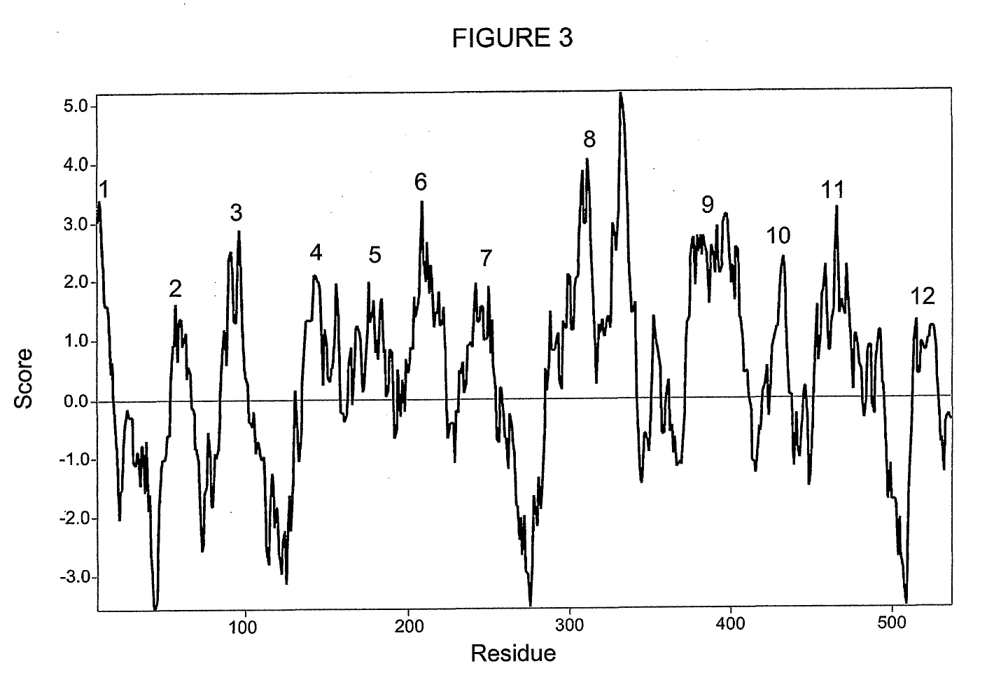

Also preferred are P450TEC polypeptide and polynucleotide fragments characterized by structural or functional domains. As set out in FIGS. 3 and 4, such preferred regions include Parker antigenic index regions and functional domains, respectively. Moreover, polynucleotide fragments encoding these domains are also contemplated.

Other preferred fragments are biologically active P450TEC fragments. Biologically active fragments are those exhibiting activity similar, but not necessarily identical, to an activity of the P450TEC polypeptide. The biological activity of the fragments may include an improved desired activity, or a decreased undesirable activity. Thus, polypeptide fragments of SEQ ID NO:17 falling within conserved domains, predicted functional domains or cytochrome benchmarks are specifically contemplated by the present invention. (See FIG. 4.)

These domains include: the N-terminal membrane-anchor sequence from about amino acid 1 to about amino acid 59; the N-terminal hydrophobic proline rich region from about amino acid 60 to about amino acid 72; a C-helix region benchmark from about amino acid 86 to about amino acid 90; an ancient eukaryotic P450 tripeptide from about amino acid 113 to about amino acid 115; an E-helix domain benchmark from about amino acid 157 to about amino acid 159; an I-helix region—oxygen binding domain from about amino acid 352 to about amino acid 356; an I-helix/J-helix junction benchmark at about amino acid 372; a J-helix benchmark at about amino acid 387; a K-helix benchmark from about amino acid 409 to about amino acid 412; the ferredoxin binding domain from about amino acid 434 to about amino acid 439; the meander region from about amino acid 456 to about amino acid 467; a heme-thiolate ligand binding domain from about amino acid 483 to about amino acid 492; a conserved tetrapeptide from about amino acid 508 to about amino acid 511. The P450TEC C terminus is at amino acid 544. Any alteration within these domains could be expected to change the functional activity or functional activities of the P450TEC protein. Thus, in another embodiment, the present invention is directed to an isolated polynucleotide which encodes fragments from about amino acid number 1 to 59, 60 to 72, 86 to 90, 113 to 115, 157 to 159, 352 to 356, 372, 387, 409 to 412, 434 to 439, 456 to 467, 483 to 492, and 508 to 511 of the amino acid sequence of SEQ ID NO:17. Preferably, said isolated polynucleotide is not AF090434 (SEQ ID NO:53) and AF090435 (SEQ ID NO:54).

V. Epitopes & Antibodies

In the present invention, “epitopes” refer to P450TEC polypeptide fragments having antigenic or immunogenic activity in an animal. A preferred embodiment of the present invention relates to a P450TEC polypeptide fragment comprising an epitope, as well as the polynucleotide encoding this fragment. A region of a protein molecule to which an antibody can bind is defined as an “antigenic epitope”. In contrast, an “immunogenic epitope” is defined as a part of a protein that elicits an antibody response. (See, for instance, Geysen et al., Proc. Nati. Acad. Sci. USA 81:3998-4002 (1983)).

Fragments which function as epitopes may be produced by any conventional means. (See, e.g., Houghten, R. A., Proc. NatI. Acad. Sci. USA 82:5131-5135 (1985), further described in U.S. Pat. No. 4,631,211.)

In the present invention, antigenic epitopes preferably contain a sequence of at least seven, more preferably at least nine, and most preferably between about 15 to about 30 amino acids. Antigenic epitopes are useful to raise antibodies, including monoclonal antibodies, that specifically bind the epitope. (See, for instance, Wilson et al., Cell 37:767-778 (1984); Sutcliffe, J. G. et al., Science 219:660-666 (1983).)

Similarly, immunogenic epitopes can be used to induce T cells according to methods well known in the art. (See, for instance, U.S. patent application No. 08/935,377, the entire contents of which are incorporated herein by reference; see also Sutcliffe et al., supra; Wilson et al., supra; Chow, M. et al., Proc. Nati. Acad. Sci. USA 82:910-914; and Bittle, F. J. et al., J. Gen. Virol. 66:2347-2354 (1985).) A preferred immunogenic epitope includes the secreted protein. The immunogenic epitopes may be presented together with a carrier protein, such as an albumin, to an animal system (such as rabbit or mouse) or, if it is long enough (at least about 25 amino acids), without a carrier. However, immunogenic epitopes comprising as few as 8 to 10 amino acids have been shown to be sufficient to raise antibodies capable of binding to, at the very least, linear epitopes in a denatured polypeptide (e.g., in Western blotting.) Using the Parker method, SEQ ID NO:17 was found to be antigenic at amino acids: M1-R19, R54-P67, S85-l101, S135 V159, K163-G191, K198-1224,1231-F254, F284-L342, N371-Q411, H423-P437,R451-Q494, and A512-F528 (see FIG. 3). Thus, these regions could be used as epitopes to produce antibodies against the protein encoded by SEQ ID NO:17.

As used herein, the term “antibody” (Ab) or “monoclonal antibody” (mAb) is meant to include intact molecules as well as antibody fragments (such as, for example, Fab and F(ab′)2 fragments) which are capable of specifically binding to protein. Such fragments lack the Fc fragment of intact antibody and are typically produced by proteolytic cleavage using enzymes such as papain (to produce Fab fragments) or pepsin (to produce F(ab′)2 fragments). Fab and F(ab′)2 fragments clear more rapidly from the circulation and may have less non-specific tissue binding than an intact antibody. (Wahl et al., I Nucl. Med. 24:316-325 (1983).) Thus, these fragments are preferred, as well as the products of a Fab or other immunoglobulin expression library. Moreover, antibodies of the present invention include chimeric, single chain, and humanized antibodies. Alternatively, target protein-binding fragments can be produced through the application of recombinant DNA technology or through synthetic chemistry.

Standard reference works setting forth general principles of immunology include Current Protocols in Immunology, John Wiley & Sons, New York; Klein, J., Immunology: The Science of Self-Nonself Discrimination, John Wiley & Sons, New York (1982); Kennett, R., et al., eds., Monoclonal Antibodies, Hybridonia: A New Dimension in Biological Analyses, Plenum Press, New York (1980); Campbell, A., “Monoclonal Antibody Technology” in Burden, R., et al., eds., Laboratory Techniques in Biochemistry and Molecular Biology, Vol. 13, Elsevere, Amsterdam (1984).

Antibodies generated against a target epitope can be obtained by direct injection of the epitope or polypeptide into an animal or by administering the polypeptides to an animal, preferably a nonhuman. The antibody so obtained will then bind the polypeptide itself. In this manner, even a sequence encoding only a fragment of the polypeptide can be used to generate antibodies binding the whole native polypeptide. Such antibodies can then be used to isolate the polynucleotide encoding the polypeptide from an expression library using the method of the present invention.

For preparation of monoclonal antibodies, any technique which provides antibodies produced by continuous cell line cultures can be used. Examples include the hybridoma technique (Kohler and Milstein, Nature 256:495-497 (1975)), the trioma technique, the human B cell hybridoma technique (Kozbor et al., Immunol. Today 4:72 (1983)), and the EBV-hybridoma technique to produce human monoclonal antibodies (Cole et al., in Monoclonal Antibodies and Cancer Therapy, Alan R. Liss, Inc., pp. 77-96 (1985)).

Techniques described for the production of single chain antibodies (U.S. Pat. 4,946,778) can be adapted to produce single chain antibodies to immunogenic polypeptide products of interest.

The antibodies useful in the present invention may be prepared by any of a variety of methods. For example, cells expressing the target protein or an antigenic fragment thereof can be administered to an animal in order to induce the production of sera containing polyclonal antibodies. In another method, a preparation of target protein is prepared and purified to render it substantially free of natural contaminants. Such a preparation is then introduced into an animal in order to produce polyclonal antisera of greater specific activity.

In a highly preferred method, antibodies useful in the present invention are monoclonal antibodies (or target protein-binding fragments thereof). Such monoclonal antibodies can be prepared using hybridoma technology (Kohler et al., Nature 256:495 (1975); Kohler et al., Eur. J. Immunol. 6:511 (1976); Kohler et al., Eur. J. Immunol. 6:292 (1976); Hammerling et al., In: Monoclonal Antibodies and T-Cell Hybridornas, Elsevier, N.Y., pp. 563-681 (1981)). In general, such procedures involve immunizing an animal (preferably a mouse) with a target protein antigen or, more preferably, with a target protein-expressing cell. Suitable cells can be recognized by their capacity to bind an anti-target protein antibody. Such cells may be cultured in any suitable tissue culture medium; however, it is preferable to culture cells in Earle's modified Eagle's medium supplemented with 10% fetal bovine serum (inactivated at about 56° C.), and supplemented with about 10 g/l of nonessential amino acids, about 1,000 U/ml of penicillin, and about 100 g/ml of streptomycin. The splenocytes of immunized mice are extracted and fused with a suitable myeloma cell line. Any suitable myeloma cell line may be employed in accordance with the present invention; however, it is preferable to employ the parent myeloma cell line (SP20), available from the American Type Culture Collection, Mannassas, Va. After fusion, the resulting hybridoma cells are selectively maintained in HAT medium, and then cloned by limiting dilution as described by Wands et al. (Gastroenterology 80:225-232 (1981)). The hybridoma cells obtained through such a selection are then assayed to identify clones which secrete antibodies capable of binding the target protein antigen.

Alternatively, additional antibodies capable of binding to the target protein antigen may be produced in a two-step procedure through the use of anti-idiotypic antibodies. Such a method makes use of the fact that antibodies are themselves antigens, and that, therefore, it is possible to obtain an antibody which binds to a second antibody. In accordance with this method, target-protein specific antibodies are used to immunize an animal, preferably a mouse. The splenocytes of such an animal are then used to produce hybridoma cells, and the hybridoma cells are screened to identify clones which produce an antibody whose ability to bind to the target protein-specific antibody can be blocked by the target protein antigen. Such antibodies comprise anti-idiotypic antibodies to the target protein-specific antibody and can be used to immunize an animal to induce formation of further target protein-specific antibodies.

In a preferred embodiment, the antibody or antibody fragment is conjugated with a toxic agent which kills cells that express a target protein. Toxic agents useful in the invention include toxins (e.g. an enzymatically active toxin of bacterial, fungal, plant or animal origin or fragments thereof). Examples of suitable toxins include diphtheria toxin, ricin, and cholera toxin.

Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha sarcin, Aleurites fordii proteins, dianthin proteins, Phytolaca americana proteins (PAPI, PAPII and PAP-S), Momordica charantia inhibitor, curin, crotin, Sapaonaria officinalis inhibitor, gelonin, mitogellin, restrictocin, phenomycin, enomycin and the tricothecenes.

Further suitable labels for the target protein-specific antibodies of the present invention are provided below. Examples of suitable enzyme labels include malate dehydrogenase, staphylococcal nuclease, delta-5-steroid isomerase, yeast-alcohol dehydrogenase, alpha-glycerol phosphate dehydrogenase, triose phosphate isomerase, peroxidase, alkaline phosphatase, asparaginase, glucose oxidase, beta-galactosidase, ribonuclease, urease, catalase, glucose-6-phosphate dehydrogenase, glucoamylase, and acetylcholine esterase.

Examples of suitable radioisotopic labels include 3H, 111 In, 125I, 131I 32P, 35S, 14C, 51Cr, 57To, 58Co, 59Fe, 75Se, 152Eu, 90Y, 67CU, 217Ci, 211At, 212Pb, 47SC, 109Pd, etc. Examples of suitable non-radioactive isotopic labels include 157Gd, 55Mn, 162Dy, 52Tr, and 56Fe.

Examples of suitable fluorescent labels include an 152Eu label, a fluorescein label, an isothiocyanate label, a rhodamine label, a phycoerythrin label, a phycocyanin label, an allophycocyanin label, an o-phthaldehyde label, and a fluorescamine label.

Examples of chemiluminescent labels include a luminal label, an isoluminal label, an aromatic acridinium ester label, an imidazole label, an acridinium salt label, an oxalate ester label, a luciferin label, a luciferase label, and an aequorin label.

Examples of nuclear magnetic resonance contrasting agents include heavy metal nuclei such as Gd, Mn, and iron.

Typical techniques for binding the above-described labels to antibodies are provided by Kennedy et al., Clin. Chim. Acta 70:1-31 (1976), and Schurs et al., Clin. Chinz. Acta 81:1-40 (1977). Coupling techniques mentioned in the latter are the glutaraldehyde method, the periodate method, the dimaleimide method, the m maleimidobenzyl-N-hydroxy-succinimide ester method, all of which methods are incorporated by reference herein.

Conjugates of the antibody and cytotoxic agent are made using a variety of bifunctional protein coupling agents such as N-succinimidyl-3 (2-pyridyidithiol) proprionate (SPDP), iminothiolane (IT), bifunctional derivatives of imidoesters (such as dimethyl adipimidate HCL), active esters (such as disucciruimidyl suberate), aldehydes (such as dimethyl adipimidate HCL), active esters (such as disuccinimidyl suberate), aldehydes (such as glutaraldehyde), bis-azido compounds (such as bis-p (azidobenzoyl) hexanediamine), bisdiazonium derivatives (such as bis-p(diazoniumbenzoyl)-ethylenediamine), diisocyantes (such as toluene 2,6-diisocyanate), and bisactive fluorine compounds (such as 1,5-difluoro-2,4-dinitrobenzene). For example, a ricin immunotoxin can be prepared as described in Vitetta et al., Science 238: 1098 (1987). 14C-labeled 1-isothiocyanatobenzyl-3 methyldiethylene triaminepentaacetic acid (MX-DTPA) is an exemplary chelating agent for conjugation of radionucleotide to the antibody. See WO 94/11026.

For in vivo use of antibodies in humans, it may be preferable to use “humanized” chimeric monoclonal antibodies. Such antibodies can be produced using genetic constructs derived from hybridoma cells producing the monoclonal antibodies described above. Methods for producing chimeric antibodies are known in the art. (See, for review, Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).)

VI. Disease State Diagnosis and Prognosis

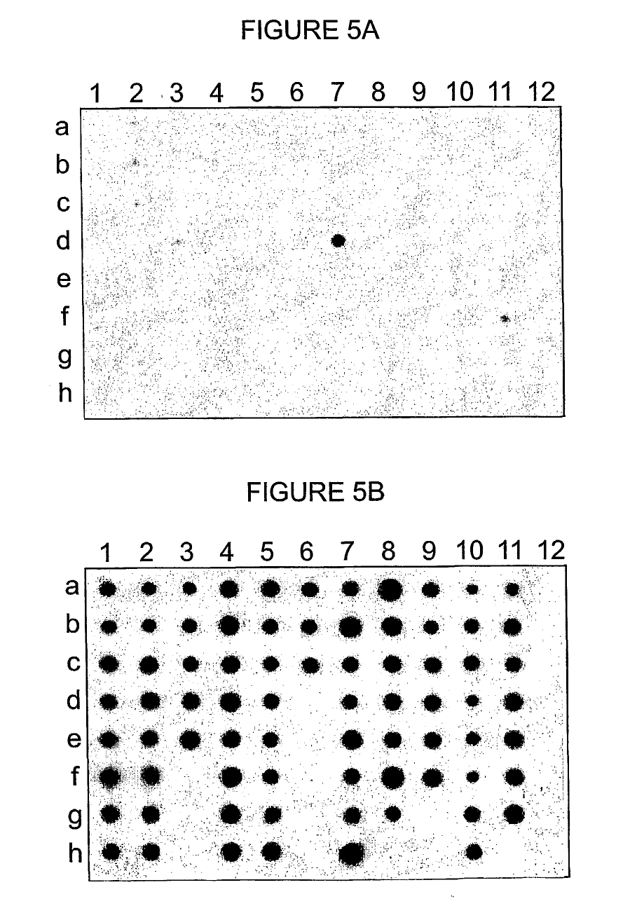

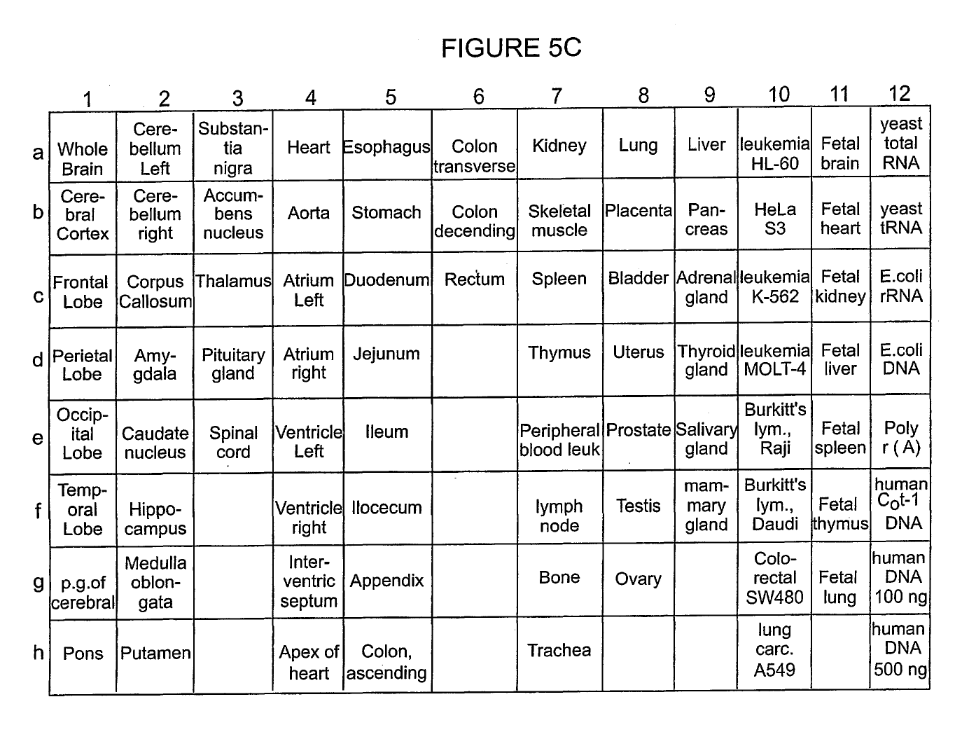

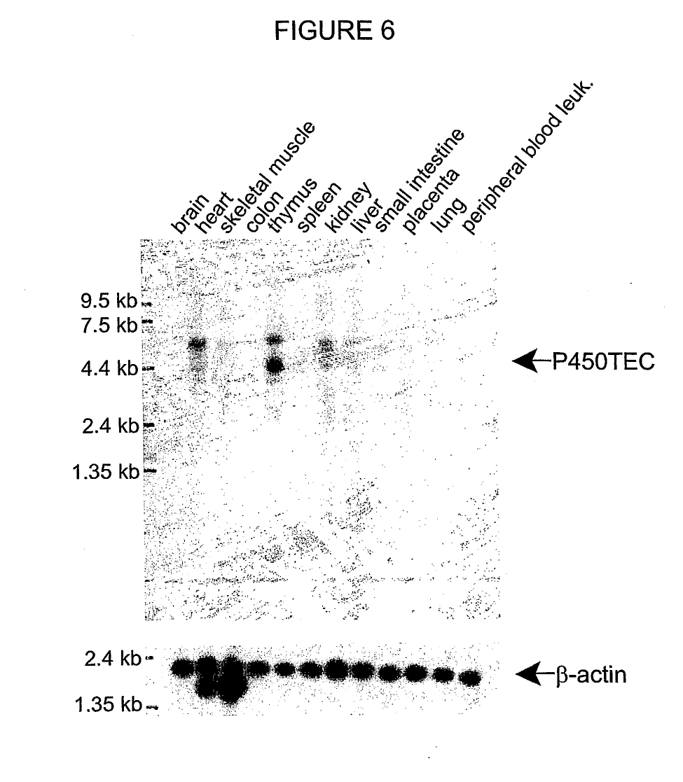

It is believed that certain maladies may cause mammals to express significantly altered levels of the P450TEC protein and mRNA encoding the P450TEC protein when compared to a corresponding “standard” mammal, i.e., a mammal of the same species not having the malady or condition. P450TEC is highly expressed in adult and fetal thymus (see FIGS. 5 and 6). The thymus is a primary lymphatic organ where lymphocytes of the immune system proliferate and mature. Thus, P450TEC polypeptides or polynucleotides may be useful in treating deficiencies or disorders of the immune system or the thymus, by activating or inhibiting maturation or the proliferation of immune or thymic cells. For example, a mammal suffering from a condition that causes abnormal thymic hypertrophy is expected to express altered levels of P450TEC by the thymus.

Further, it is believed that altered levels of the P450TEC protein can be detected in certain body fluids (e.g., blood, sera, plasma, urine, and spinal fluid) from mammals with such a condition when compared to sera from mammals of the same species not having the condition. Thus, the present invention provides a diagnostic method useful during diagnosis of one of the many conditions of the thymus, such as thymomas (neoplasm of thymic epithelial cells) and thymic follicular hyperplasia, as encountered in myasthenia gravis, Graves' disease, Addison's disease, SLE, scleroderma and rheumatoid arthritis. The method involves assaying the expression level of the gene encoding the P450TEC protein in mammalian cells or body fluid and comparing the gene expression level with a standard P450TEC gene expression level, whereby an alteration in the gene expression level over the standard is indicative of said conditions.

Where a diagnosis has already been made according to conventional methods, the present invention is useful as a prognostic indicator, whereby patients exhibiting altered P450TEC gene expression will experience a worse clinical outcome relative to patients expressing the gene at a normal level.

Additionally, the presence of P450TEC or mRNA level can be measured to qualitatively determine cell or tissue type. The P450TEC gene was discovered in a thymus cDNA library. Since P450TEC is highly expressed in adult and fetal thymus, P450TEC protein and mRNA expression can be used as a marker to detect thymic cells that are present in a cell culture.

By “assaying the expression level of the gene encoding the P450TEC protein” is intended qualitatively or quantitatively measuring or estimating the level of the P450TEC protein or the level of the mRNA encoding the P450TEC protein in a first biological sample either directly (e.g., by determining or estimating absolute protein level or mRNA level) or relatively (e.g., by comparing to the P450TEC protein level or mRNA level in a second biological sample).

Preferably, the P450TEC protein level or mRNA level in the first biological sample is measured or estimated and compared to a standard P450TEC protein level or mRNA level, the standard being taken from a second biological sample obtained from an individual not having the condition. As will be appreciated in the art, once a standard P450TEC protein level or mRNA level is known, it can be used repeatedly as a standard for comparison. By “biological sample” is intended any biological sample obtained from an individual, cell line, tissue culture, or other source which contains P450TEC protein or mRNA. Biological samples include mammalian body fluids (such as sera, plasma, urine, synovial fluid and spinal fluid) which contain secreted mature P450TEC protein, and thymic, heart, kidney, left and right cerebellum, corpus callosum, aorta and pituitary tissue. Methods for obtaining tissue biopsies and body fluids from mammals are well known in the art. Where the biological sample is to include mRNA, a tissue biopsy is the preferred source.

Preferred mammals include monkeys, apes, cats, dogs, cows, pigs, horses, rabbits and humans. Particularly preferred are humans.

Total cellular RNA can be isolated from a biological sample using any suitable technique such as the single-step guanidinium thiocyanate-phenol chloroform method described in Chomczynski and Sacchi, Anal. Biochem.162:156-159 (1987). Levels of mRNA encoding the P450TEC protein are then assayed using any appropriate method. These include Northern blot analysis (Harada et al., Cell 63:303-312 (1990)),S1 nuclease mapping (Fujita et al., Cell 49:357-367 (1987)), the polymerase chain reaction (PCR), reverse transcription in combination with the polymerase chain reaction (RT-PCR) (Makino et al., Technique 2:295-301 (1990)), and reverse transcription in combination with the ligase chain reaction (RT-LCR).

Assaying P450TEC protein levels in a biological sample can occur using antibody-based techniques. For example, P450TEC protein expression in tissues can be studied with classical immunohistological methods (Jalkanen, M., et al., J. Cell. Biol. 101: 976-985 (1985); Jalkanen, M., et al., J. Cell. Biol. 105:30873096 (1987)).

Other antibody-based methods useful for detecting P450TEC protein gene expression include immunoassays, such as enzyme linked immunosorbent assay (ELISA) and the radioimmunoassay (RIA).

Suitable labels are known in the art and include enzyme labels, such as, glucose oxidase, and radioisotopes, such as iodine (125I, 121I), carbon (14C), sulfur (35S), tritium (3H), indium (112In), and technetium (99mTc), and fluorescent labels, such as fluorescein and rhodarnine, and biotin.

In addition to assaying secreted protein levels in a biological sample, proteins can also be detected in vivo by imaging. Antibody labels or markers for in vivo imaging of protein include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the antibody by labeling of nutrients for the relevant hybridoma.

A protein-specific antibody or antibody fragment which has been labeled with an appropriate detectable imaging moiety, such as a radioisotope (for example, 131I, 112In, 99mTc), a radio-opaque substance, or a material detectable by nuclear magnetic resonance, is introduced (for example, parenterally, subcutaneously, or intraperitoneally) into the mammal. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of 99mTc. The labeled antibody or antibody fragment will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).)

VII. P450TEC Fusion Proteins

Any P450TEC polypeptide can be used to generate fusion proteins. For example, the P450TEC polypeptide, when fused to a second protein, can be used as an antigenic tag. Antibodies raised against the P450TEC polypeptide can be used to indirectly detect the second protein by binding to the P450TEC polypeptide. Moreover, because secreted proteins target cellular locations based on trafficking signals, the P450TEC polypeptide can be used as a targeting molecule once fused to other proteins.

Examples of domains that can be fused to P450TEC polypeptides include not only heterologous signal sequences, but also other heterologous functional regions. The fusion does not necessarily need to be direct, but may occur through linker sequences.

In certain preferred embodiments, P450TEC fusion polypeptides may be constructed which include additional N-terminal and/or C-terminal amino acid residues. In particular, any N-terminally or C-terminally deleted P450TEC polypeptide disclosed herein may be altered by inclusion of additional amino acid residues at the N-terminus to produce a P450TEC fusion polypeptide. In addition, P450TEC fusion polypeptides are contemplated which include additional N terminal and/or C-terminal amino acid residues fused to a P450TEC polypeptide comprising any combination of N- and C terminal deletions set forth above.

Moreover, fusion proteins may also be engineered to improve characteristics of the P450TEC polypeptide. For instance, a region of additional amino acids, particularly charged amino acids, may be added to the N-terminus of the P450TEC polypeptide to improve stability and persistence during purification from the host cell or subsequent handling and storage. Also, peptide moieties may be added to the P450TEC polypeptide to facilitate purification. Such regions may be removed prior to final preparation of the P450TEC polypeptide. The addition of peptide moieties to facilitate handling of polypeptides are familiar and routine techniques in the art.

Moreover, P450TEC polypeptides, including fragments, and specifically epitopes, can be combined with parts of the constant domain of immunoglobulins (IgG), resulting in chimeric polypeptides. These fusion proteins facilitate purification and show an increased half life in vivo. One reported example describes chimeric proteins consisting of the first two domains of the human CD4-polypeptide and various domains of the constant regions of the heavy or light chains of mammalian immunoglobulins. (EP A394,827; Traunecker et al., Nature 331:84-86 (1988).) Fusion proteins having disulfide-linked dimeric structures (due to the IgG) can also be more efficient in binding and neutralizing other molecules, than the monomeric secreted protein or protein fragment alone. (Fountoulakis et al., J. Biochem. 270:3958-3964 (1995).)

Similarly, EP-A-0 464 533 (Canadian counterpart 2045869) discloses fusion proteins comprising various portions of constant region of immunoglobulin molecules together with another human protein or part thereof. In many cases, the Fc part in a fusion protein is beneficial in therapy and diagnosis, and thus can result in, for example, improved pharmacokinetic properties. (EP-A 0232 262.) Alternatively, deleting the Fc part after the fusion protein has been expressed, detected, and purified, would be desired. For example, the Fc portion may hinder therapy and diagnosis if the fusion protein is used as an antigen for immunizations. In drug discovery, for example, human proteins, such as hlL-5, have been fused with Fc portions for the purpose of high-throughput screening assays to identify antagonists of hIL-5. (See, D. Bennett et al., J. Molecular Recognition 8:52-58 (1995); K. Johanson et al., J. Biol. Chem. 270:9459-9471 (1995).)

Moreover, the P450TEC polypeptides or fragments can be fused to other proteins, e.g., ferredoxin reductase, other flavoproteins or other proteins which may function as a cytochrome P450TEC reductase or facilitate such an activity to create a multiprotein fusion complex. Such a multiprotein fusion complex may function as an enzymatically active covalently linked P450TEC-reductase complex. A multiprotein complex can be synthesized by the means of chemical crosslinking or assembled via novel intramolecular interactions, e.g., by the use of specific antibodies stabilizing the complex.

Moreover, the P450TEC polypeptides can be fused to lipophilic molecules, including but not limited to fatty acids, whereby the lipophilic molecules are used to stabilize P450TEC interactions with other proteins, natural membranes or artificial membranes, or to create new interactions with other proteins, natural membranes or artificial membranes. Fusion of the P450TEC polypeptides to lipophilic molecules can also be used to change its solubility.

Moreover, the P450TEC polypeptide can be fused to hydrophilic molecules, including but not limited to polyethylene glycol and modified oligosaccharides and polysaccharides, whereby the hydrophilic molecules are used to stabilize P450TEC interactions with other proteins, natural membranes, or artificial membranes, or to create new interactions with other proteins, natural membranes, or artificial membranes. Fusion of the P450TEC polypeptides to hydrophilic molecules can also be used to change its solubility.

Moreover, P450TEC polypeptide variants which contain non-standard amino acids or additional chemical modifications which have use in purification, stabilization or identification of the resulting modified P450TEC protein, or influence its other properties such as enzymatic activity or interaction with other proteins, membranes, solid supports or chromatographic resin are contemplated. This includes, but is not limited to, biotinylated derivatives or fusions of P450TEC polypeptides.

Moreover, modifications of the P450TEC nucleotide sequence include those where relevant regions of the P450TEC gene or polypeptide are inserted into another gene sequence to create a chimeric protein with a desired activity (enzymatic or otherwise). Such chimeric proteins can be obtained by, for example, replacing regions of other cytochrome P450 genes or polypeptides with a relevant P450TEC region whereby such a modification confers a new functional property to the resulting chimeric protein, including but not limited to new specificity, changed enzymatic kinetics, new or changed interactions with reductase or other relevant molecules or membranes, changed solubility and changed stability.

Moreover, the P450TEC polypeptides can be fused to marker sequences, such as a peptide which facilitates purification of P45OTEC. In preferred embodiments, the marker amino acid sequence is a hexa-histidine peptide (Histag), such as the tag provided in a pQE vector (QIAGEN, Inc., 9259 Eton Avenue, Chatsworth, Calif., 91311), among others, many of which are commercially available. As described in Gentz et al., Proc. And. Acad. Sci. USA 86:821-824 (1989), for instance, hexa-histidine provides for convenient purification of the fusion protein. Another peptide tag useful for purifcation, the “HA” tag, corresponds to an epitope derived from the influenza hemagglutinin protein. (Wilson et al., Cell 37:767 (1984).) The P450TEC polypeptide can also be fused to glutathione S-transferase (GST).

Thus, any of these above fusions can be engineered using the P450TEC polynucleotides or the P450TEC polypeptides.

VIII. Vectors, Host Cells, and Protein Production

The present invention also relates to vectors containing the P450TEC polynucleotide, host cells, and the production of P450TEC polypeptides by recombinant techniques. The vector may be, for example, a phage, plasmid, viral, or retroviral vector. Retroviral vectors may be replication competent or replication defective. In the latter case, viral propagation generally will occur only in complementing host cells.

P450TEC polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The P450TEC polynucleotide insert should be operatively linked to an appropriate promoter, such as the phage lambda PL promoter, the E. coli lac, trp, phoA and tac promoters, the SV40 early and late promoters and promoters of retroviral LTRs, to name a few. Other suitable promoters will be known to the skilled artisan. The expression constructs will further contain sites for transcription initiation, termination, and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS, 293, and Bowes melanoma cells; and plant cells. Appropriate culture mediums and conditions for the above-described host cells are known in the art.

Among vectors preferred for use in bacteria include pHE-4 (and variants thereof); pQE70, pQE60 and pQE-9, available from QIAGEN, Inc.; pBluescript vectors, Phagescript vectors, pNH8A, pNH16a, pNH18A, pNH46A, available from Stratagene Cloning Systems, Inc.; ptrc99a, pKK223-3, pKK233-3, pDR540, pRIT5 available from Pharmacia Biotech, Inc.; and pET28A+and pET24A+from Novogen, Inc. Among preferred eukaryotic vectors are pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan. Preferred vectors are poxvirus vectors, particularly vaccinia virus vectors such as those described in U.S. patent application No. 08/935,377, the entire contents of which are incorporated herein by reference.

Introduction of the construct into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al., Basic Methods in Molecular Biology (1986). It is specifically contemplated that P450TEC polypeptides may in fact be expressed by a host cell lacking a recombinant vector.

P450TEC polypeptides can be recovered and purified from recombinant cell cultures by well-known methods including amonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) may be employed for purification.

P450TEC polypeptides can also be recovered from: products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect, and mammalian cells. Depending upon the host employed in a recombinant production procedure, the P450TEC polypeptides may be glycosylated or may be nonglycosylated. In addition, P450TEC polypeptides may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked.

In addition to encompassing host cells containing the vector constructs discussed herein, the invention also encompasses primary, secondary, and immortalized host cells of vertebrate origin, particularly mammalian origin, that have been engineered to delete or replace endogenous genetic material (e.g.

P450TEC coding sequence), and/or to include genetic material (e.g., heterologous polynucleotide sequences) that is operably associated with P450TEC polynucleotides of the invention, and which activates, alters, and/or amplifies endogenous P450TEC polynucleotides. For example, techniques known in the art may be used to operably associate heterologous control regions (e.g., promoter and/or enhancer) and endogenous P450TEC polynucleotide sequences via homologous recombination (see, e.g., U.S. Pat. No. 5,641,670, issued Jun. 24, 1997; International Publication No. WO 96/29411, published Sep. 26, 1996; International Publication No. WO 94/12650, published Aug. 4, 1994; Koller et al., Proc. Natl. Acad. Sci. USA 86:8932 8935 (1989); and Zijlstra et al., Nature 342:435-438 (1989), the disclosures of each of which are incorporated by reference in their entireties).

IX. Uses of P450TEC Polynucleotides

The P450TEC polynucleotides identified herein can be used in numerous ways as reagents. The following description should be considered exemplary and utilizes known techniques.

There exists an ongoing need to identify new chromosome markers, since few chromosome marking reagents, based on actual sequence data (repeat polymorphisms), are presently available.

Briefly, sequences can be mapped to chromosomes by preparing PCR primers (preferably 15-25 bp) from the sequence shown in SEQ ID NO:16 (FIG. 1A). Primers can be selected using computer analysis so that primers do not span more than one predicted exon in the genomic DNA. These primers are then used for PCR screening of somatic cell hybrids containing individual human chromosomes. Only those hybrids containing the human P450TEC gene corresponding to SEQ ID NO:16 will yield an amplified fragment.

Similarly, somatic hybrids provide a rapid method of PCR mapping the polynucleotides to particular chromosomes. Three or more clones can be assigned per day using a single thermal cycler. Moreover, sublocalization of the P450TEC polynucleotides can be achieved with panels of specific chromosome fragments. Other gene mapping strategies that can be used include in situ hybridization, prescreening with labeled flow-sorted chromosomes, and preselection by hybridization to construct chromosome specific cDNA libraries.

Precise chromosomal location of the P450TEC polynucleotides can also be achieved using fluorescence in situ hybridization (FISH) of a metaphase chromosomal spread. This technique uses polynucleotides as short as 500 or 600 bases; however, polynucleotides 2,000-4,000 bp are preferred. For a review of this technique, see Verma et al., “Human Chromosomes: a Manual of Basic Techniques,” Pergamon Press, New York (1988).

For chromosome mapping, the P450TEC polynucleotides, can be used individually (to mark a single chromosome or a single site on that chromosome) or in panels (for marking multiple sites and/or multiple chromosomes). Preferred polynucleotides correspond to the noncoding regions of the cDNAs because the coding sequences are more likely conserved within gene families, thus increasing the chance of cross hybridization during chromosomal mapping.

Once a polynucleotide has been mapped to a precise chromosomal location, the physical position of the polynucleotide can be used in linkage analysis. Linkage analysis establishes coinheritance between a chromosomal location and presentation of a particular disease. (Disease mapping data are found, for example, in V. McKusick, Mendelian Inheritance in Man (available online through Johns Hopkins University Welch Medical Library).) Assuming 1 megabase mapping resolution and one gene per 20 kb, a cDNA precisely localized to a chromosomal region associated with the disease could be one of 50-500 potential causative genes.

Thus, once coinheritance is established, differences in the P450TEC polynucleotide and the corresponding gene between affected and unaffected individuals can be examined. First, visible structural alterations in the chromosomes, such as deletions or translocations, are examined in chromosome spreads or by PCR. If no structural alterations exist, the presence of point mutations are ascertained. Mutations observed in some or all affected individuals, but not in normal individuals, indicates that the mutation may cause the disease. However, complete sequencing of the P450TEC polypeptide and the corresponding gene from several normal individuals is required to distinguish the mutation from a polymorphism. If a new polymorphism is identified, this polymorphic polypeptide can be used for further linkage analysis. The presence of a polymorphism can also be indicative of a disease or a predisposition to a disease. Thus, a method of diagnosis of a P450TEC-related disease or a predisposition to a P450TEC-related disease, by identifying a polymorphism in a P450TEC gene, is also contemplated by the inventors. In addition, a diagnostic kit for identification of polymorphisms in the P450TEC gene by screening the P450TEC gene from a human for polymorphisms is also an object of the present invention.

Furthermore, increased or decreased expression of the gene in affected individuals as compared to unaffected individuals can be assessed using P450TEC polynucleotides. Any of these alterations (altered expression, chromosomal rearrangement, or mutation) can be used as a diagnostic or prognostic marker.

In addition to the foregoing, a P450TEC polynucleotide can be used to control gene expression through triple helix formation or antisense DNA or RNA. Both methods rely on binding of the polynucleotide to DNA or RNA. For these techniques, preferred polynucleotides are usually 20 to 40 bases in length and complementary to either the region of the gene involved in transcription (triple helix—see Lee et al., Nucl. Acids Res. 6:3073 (1979); Cooney et al., Science 241:456 (1988); and Dervan et al., Science 251:1360 (1991)) or to the mRNA itself (antisense—Okano, J. Neurochem. 56:560 (1991); Oligodeoxy-nucleotides as Antisense Inhibitors of Gene Expression, CRC Press, Boca Raton, Fla. (1988).) Triple helix formation optimally results in a shut-off of RNA transcription from DNA, while antisense RNA hybridization blocks translation of an mRNA molecule into polypeptide. Both techniques are effective in model systems, and the information disclosed herein can be used to design antisense or triple helix polynucleotides in an effort to treat disease.

P450TEC polynucleotides are also useful in gene therapy. One goal of gene therapy is to insert a normal gene into an organism having a defective gene, in an effort to correct the genetic defect. P450TEC offers a means of targeting such genetic defects in a highly accurate manner. Thus, for example, cells removed from a patient can be engineered with a P450TEC polynucleotide (DNA or RNA) encoding a P450TEC polypeptide ex vivo, with the engineered cells then being infused back into a patient to be treated with the polypeptides. Such methods are well-known in the art. For example, cells can be engineered by procedures known in the art by use of a retroviral particle containing RNA encoding the polypeptides of the present invention.

Another goal of gene therapy is to insert a new gene that was not present in the host genome, thereby producing a new trait in the host cell.

The P450TEC polynucleotides are also useful for identifying individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identifying personnel. This method does not suffer from the current limitations of “DogTags” which can be lost, switched, or stolen, making positive identification difficult. The P450TEC polynucleotides can be used as additional DNA markers for RFLP.

The P450TEC polynucleotides can also be used as an alternative to RFLP, by determining the actual base-by-base DNA sequence of selected portions of an individual's genome. These sequences can be used to prepare PCR primers for amplifying and isolating such selected DNA, which can then be sequenced. Using this technique, individuals can be identified because each individual will have a unique set of DNA sequences. Once an unique ID database is established for an individual, positive identification of that individual, living or dead, can be made from extremely small tissue samples.

Forensic biology also benefits from using DNA-based identification techniques as disclosed herein. DNA sequences taken from very small biological samples such as tissues, e.g., hair or skin, or body fluids, e.g., blood, saliva, semen, urine, etc., can be amplified using PCR. In one prior art technique, gene sequences amplified from polymorphic loci, such as DQa class 11 HLA gene, are used in forensic biology to identify individuals. (Erlich, H., PCR Technology, Freeman and Co. (1992).) Once these specific polymorphic loci are amplified, they are digested with one or more restriction enzymes, yielding an identifying set of bands on a Southern blot probed with DNA corresponding to the DQa class 11 HLA gene. Similarly, P450TEC polynucleotides can be used as polymorphic markers for forensic purposes.

There is also a need for reagents capable of identifying the source of a particular tissue. Such need arises, for example, in forensics when presented with tissue of unknown origin. Appropriate reagents can comprise, for example, DNA probes or primers specific to particular tissue prepared from P450TEC sequences. Panels of such reagents can identify tissue by species and/or by organ type. In a similar fashion, these reagents can be used to screen tissue cultures for contamination.

Because P450TEC is found expressed in adult and fetal thymus, heart, kidney, left and right cerebellum, corpus callosum, pituitary and aorta, P450TEC polynucleotides are useful as hybridization probes for differential identification of the tissue(s) or cell type(s) present in a biological sample. Similarly, polypeptides and antibodies directed to P450TEC polypeptides are useful to provide immunological probes for differential identification of the tissue(s) or cell type(s). In addition, for a number of disorders of the above tissues or cells, significantly higher or lower levels of P450TEC gene expression may be detected in certain tissues (e.g., cancerous and wounded tissues) or bodily fluids (e.g., serum, plasma, urine, synovial fluid or spinal fluid) taken from an individual having such a disorder, relative to a “standard” P450TEC gene expression level, i.e., the P450TEC expression level in healthy tissue from an individual not having the disorder.

Thus, the invention provides a diagnostic method of a disorder, which involves: (a) assaying P450TEC gene expression level in cells or body fluid of an individual; (b) comparing the P450TEC gene expression level with a standard P450TEC gene expression level, whereby an increase or decrease in the assayed P450TEC gene expression level compared to the standard expression level is indicative of a disorder.

In the very least, the P450TEC polynucleotides can be used as molecular weight markers on Southern gels, as diagnostic probes for the presence of a specific mRNA in a particular cell type, as a probe to “subtract-out” known sequences in the process of discovering novel polynucleotides, for selecting and making oligomers for attachment to a “gene chip” or other support, to raise anti-DNA antibodies using DNA immunization techniques, and as an antigen to elicit an immune response.

X. Uses of P450TEC Polypeptides

P450TEC polypeptides can be used in numerous ways. The following description should be considered exemplary and utilizes known techniques.

P450TEC polypeptides can be used to assay protein levels in a biological sample using antibody-based techniques. For example, protein expression in tissues can be studied with classical immunohistological methods. (Jalkanen, M., et al., J. Cell. Biol. 101:976-985 (1985); Jalkanen, M. et al., J. Cell. Biol. 105:3087 3096 (1987).) Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes, such as iodine (125I, 121I), carbon (14C), sulfur (35S), tritium (3H), indium (12In), and technetium (99mTc), and fluorescent labels, such as fluorescein and rhodamine, and biotin.

Moreover, P450TEC polypeptides can be used to treat disease. For example, patients can be administered P450TEC polypeptides in an effort to replace absent or decreased levels of the P450TEC polypeptide (e.g., insulin), to supplement absent or decreased levels of a different polypeptide (e.g., hemoglobin S for hemoglobin B), to inhibit the activity of a polypeptide (e.g., an oncogene), to activate the activity of a polypeptide (e.g., by binding to a receptor), to reduce the activity of a membrane bound receptor by competing with it for free ligand (e.g., soluble TNF receptors used in reducing inflammation), or to bring about a desired response (e.g., blood vessel growth).

Similarly, antibodies directed to P450TEC polypeptides can also be used to treat disease. As described in detail in the “Epitopes and Antibodies” section supra, the polypeptides of the present invention can be used to raise polyclonal and monoclonal antibodies, which are useful in assays for detecting P450TEC protein expression from a recombinant cell, as a way of assessing transformation of the host cell, or as antagonists capable of inhibiting P450TEC protein function. Similarly, administration of an antibody can activate the polypeptide, such as by binding to a polypeptide bound to a membrane (receptor). Further, such polypeptides can be used in the yeast two-hybrid system to “capture” P450TEC protein binding proteins which are also candidate agonist and antagonist according to the present invention. The yeast two hybrid system is described in Fields and Song, Nature 340.245-246 (1989).

Small molecules that are specific substrates or metabolites of P450TEC protein can also be used in the diagnosis or analysis of disease states involving P450TEC or to monitor progress of therapy.

P450TEC or its derivatives, P450TEC fusions, complexes and chimeric proteins can also be used in the analysis of individual chemicals or complex mixtures of chemicals including, but not limited to, the screening for improved or changed small molecules. These molecules may have use in development of new therapeutic agents or new diagnostic methods for P450TEC-related disorders.

P450TEC or its derivatives, P450TEC fusions, complexes and chimeric proteins in an isolated state or as a part of complex mixtures can also be used to synthesize or modify small molecules. These molecules can in turn be used as therapeutic or diagnostic agents. Furthermore, these molecules can be used in the development of additional new molecules for therapeutic or diagnostic use.

P450TEC or its derivatives, P450TEC homologs, chimeras and protein fusions can be expressed in natural host cells or organisms, or in experimentally created cells or organisms for the purpose of producing, analyzing or modifying therapeutically and diagnostically important small molecules.

P450TEC or its derivatives and P450TEC fusions can be expressed in cells or organisms to modify the normal or diseased function and state of such hosts. In particular, this encompasses, but is not limited to, the use of P450TEC polypeptides and derivatives for gene-therapy of humans or animals. P450TEC polypeptides can also be used in experimental animals to reproduce physiological states, which are useful in the study and analysis of human disease, health or development.

P450TEC polypeptides or derivatives and P450TEC fusions can be expressed in natural host cells or organisms, or in experimentally created cells or organisms and used in the extraction, conversion, localization or bioremedation of small molecules in natural or artificial environments. This use includes, but is not limited to, the removal or neutralization of environmental or industrial pollutants by cultivating transgenic or genetically modified plants or microorganisms in water or soil, or by assembling so-called bioreactors that host such organisms.

At the very least, the P450TEC polypeptides can be used as molecular weight markers on SDS-PAGE gels or on molecular sieve gel filtration columns using methods well known to those of skill in the art. Moreover, P450TEC polypeptides can be used to test the following biological activities.

XI. Metabolism of Arachidonic Acid

P450TEC possesses homology to two piscine proteins, CYP2N1 (SEQ ID NO:53) and CYP2N2 (SEQ ID NO:54) (see FIG. 2), which have been shown to metabolize arachidonic acid to epoxyeicosatrienoic acids (EETs) (Oleksiak et al., J. Biol. Chem. 275:2312-2321 (2000)). The P450TEC polypeptide also metabolizes arachidonic acid. In a preffered embodiment, the P450 TEC of the invention hydroxylses arachidonic acid, most preferably to a HETE metabolite.

EETs, such as prostaglandins, thromboxanes and leukotrienes, have been shown to activate phosphorylase a, regulate coronary artery, intestine and cerebral vascular tone, modulate Ca2+transport, activate Ca2+-activated K+channels, and modulate the secretion of neuropeptides. The EETs have also been shown to affect general physiological processes such as cellular proliferation and tyrosine kinase activity. In addition, prostaglandins are known to stimulate inflammation, regulate blood flow to particular organs, control ion transport across membranes and modulate synaptic transmission.

P450TEC can hydroxylate arachidonic acid. Such metabolites are preferably the monohydroxylated arachidonic acid, such as 5−, 8−, 9−, 11−, 12−, 15−, 19−and 20−HETE, but are not necessarily limited to such. Such metabolites, also have effects relating to calcium, sodium and potassium ion transport and related conditions, vasoconstriction, chemotaxis, platelet aggregation, inflammation, autoimmune disorders. Effects in the immune, cardiovascular and renal systems and in the gastrointestinal tract have also been reported. A person skilled in the art would be familiar with other HETE related effects that have been identified in the art.

Thus, the P450TEC polypeptide metabolizes arachidonic acid to eicosatrienoic acids and affect several physiological processes, such as, inflammation, ion transport, and synaptic transmission.

XII. Heme-binding, Oxygen-binding and Detoxification

All cytochrome P450s are heme-binding proteins that contain the putative family signature, F(XX)G(XXX)C(X)G (X means any residue; conserved residues are in bold). The heme-binding signature in P450TEC can be found at amino acids 483-492 of SEQ ID NO:17 and contains the motif FGIGKRVCMG (see FIG. 1B).

Heme-binding proteins, such as the cytochrome P450s, play an important role in the detoxification of toxic substances or xenobiotics. For example, toxic substances can be detoxified by oxidation. Cytochrome P450s can function as oxidative enzymes to detoxify toxic substances, such as phenobarbital, codeine and morphine. The capacity of cytochrome P450s to bind oxygen depends on the presence of a heme group and the oxygen binding domain. Thus, the ability of P450s to bind heme and molecular oxygen enables them to detoxify toxic substances by oxidation.

Thus, P450TEC polypeptides are also useful as oxidative enzymes to detoxify toxic substances or xenobiotics, such as phenobarbital, codeine and morphine.

XIII. Immune Activity

P450TEC is highly expressed in adult and fetal thymus (see FIGS. 5 and 6). The thymus is a primary lymphatic organ where lymphocytes of the immune system proliferate and mature. Thus, P450TEC polypeptides or polynucleotides or anti-P450TEC antibodies are expected to be useful in treating deficiencies or disorders of the immune system, by activating or inhibiting the proliferation and maturation of immune cells.

Immune cells develop through a process called hematopoiesis, producing myeloid (platelets, red blood cells, neutrophils, and macrophages) and lymphoid (B and T lymphocytes) cells from pluripotent stem cells. The etiology of these immune deficiencies or disorders may be genetic, somatic, such as cancer or some autoimmune disorders, acquired (e.g., by chemotherapy or toxins), or infectious. Moreover, P450TEC polynucleotides or polypeptides can be used as a marker or detector of a particular immune system disease or disorder.

P450TEC polynucleotides or polypeptides or anti-P450TEC antibodies may also be useful in treating or detecting autoimmune disorders. Many autoimmune disorders result from inappropriate recognition of self as foreign material by immune cells. This inappropriate recognition results in an immune response leading to the destruction of the host tissue. Therefore, the administration of P450TEC polypeptides or polynucleotides that can inhibit an immune response, particularly the proliferation and differentiation of T-cells, may be an effective therapy in preventing autoimmune disorders.

Examples of autoimmune disorders that can be treated or detected by P450TEC include, but are not limited to: Addison's Disease, hemolytic anemia, antiphospholipid syndrome, rheumatoid arthritis, dermatitis, allergic encephalomyelitis, glomerulonephritis, Goodpasture's Syndrome, Graves' Disease, Multiple Sclerosis, Myasthenia Gravis, Neuritis, Ophthalmia, Bullous Pemphigoid, Pemphigus, Polyendocrinopathies, Purpura, Reiter's Disease, Stiff-Man Syndrome, Autoimmune Thyroiditis, Systemic Lupus Erythematosus, Autoimmune Pulmonary Inflammation, Guillain-Barre Syndrome, insulin dependent diabetes mellitis, and autoimmune inflammatory eye disease.

P450TEC polynucleotides or polypeptides or anti-P450TEC antibodies may also be used to treat and/or prevent organ rejection or graft versus-host disease (GVHD). Organ rejection occurs by host immune cell destruction of the transplanted tissue through an immune response. Similarly, an immune response is also involved in GVHD, but, in this case, the foreign transplanted immune cells destroy the host tissues. The administration of P450TEC polypeptides or polynucleotides that inhibits an immune response, particularly the proliferation, differentiation, or chemotaxis of T-cells, may be an effective therapy in preventing organ rejection or GVHD.

XIV. Hyperproliferative Disorders

P450TEC polypeptides or polynucleotides or anti-P450TEC antibodies can be used to treat or detect hyperproliferative disorders, including thymic neoplasms. P450TEC polypeptides or polynucleotides or anti-P450TEC antibodies may inhibit the proliferation of the disorder through direct or indirect interactions. Alternatively, P450TEC polypeptides or polynucleotides or anti P450TEC antibodies may proliferate other cells which can inhibit the hyperproliferative disorder.

For example, by increasing an immune response, particularly increasing antigenic qualities of the hyperproliferative disorder or by proliferating, differentiating, or mobilizing T-cells, hyperproliferative disorders can be treated. This immune response may be increased by either enhancing an existing immune response, or by initiating a new immune response. Alternatively, decreasing an immune response may also be a method of treating hyperproliferative disorders, such as a chemotherapeutic agent.

Examples of hyperproliferative disorders that can be treated or detected by P450TEC polynucleotides or polypeptides or anti-P450TEC antibodies include, but are not limited to neoplasms located in the: thymus, kidney, heart, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital.

XV. Antagonists, Agonists and Antisense Methods

This invention further provides methods for screening compounds to identify agonists and antagonists to the P450TEC polypeptides of the present invention. An agonist is a compound which has similar biological functions, or enhances the functions, of the polypeptides, while antagonists block such functions.

Labeled arachidonic acid would be incubated with P450TEC polypeptide, e.g. radioactivity, in the presence of the compound. The ability of the compound to block the catalysis of arachidonic acid by P450TEC could then be measured.

Examples of potential P450TEC antagonists include antibodies, drugs, small molecules, or in some cases, oligonucleotides, which bind to the polypeptides.

Antisense constructs prepared using antisense technology are also potential antagonists. Thus, the present invention is further directed to inhibiting P450TEC in vivo by the use of antisense technology. Antisense technology can be used to control gene expression through triple-helix formation or antisense DNA or RNA, both of which methods are based on binding of a polynucleotide to DNA or RNA. For example, the 5′ coding portion of the polynucleotide sequence, which encodes for the [mature] polypeptides of the present invention, is used to design an antisense RNA oligonucleotide of from about 10 to 40 base pairs in length. The antisense RNA oligonucleotide hybridizes to the mRNA in vivo and blocks translation of the mRNA molecule into the polypeptides (antisense—Okano, J. Neurochem. 56:560 (1991); Oligodeoxynucleotides as Antisense Inhibitors of Gene Expression, CRC Press, Boca Raton, Fla. (1988)). A DNA oligonucleotide is 30 designed to be complementary to a region of the gene involved in transcription (triple-helix, see Lee et al., Nucl. Acids Res. 6:3073 (1979); Cooney et al, Science 241:456 (1988); and Dervan et al., Science 251:1360 (1991)), thereby preventing transcription and the production of the P450TEC polypeptides. The oligonucleotides described above can also be delivered to cells such that the antisense RNA or DNA may be expressed in vivo to inhibit production of the chemokine polypeptides.

Another potential P450TEC antagonist is a peptide derivative of the polypeptides which are naturally or synthetically modified analogs of the polypeptides that have lost biological function yet still recognize and bind to arachidonic acid to thereby effectively block its metabolism. Examples of peptide derivatives include, but are not limited to, small peptides or peptide-like molecules.

The antagonists may be employed to treat disorders which are either P450TEC-induced or enhanced or modulated, for example, acute and chronic inflammatory diseases, including inflammation associated with infection (e.g., septic shock, sepsis, or systemic inflammatory response syndrome (SIRS)), ischemia-reperfusion injury, endotoxin lethality, arthritis, complement-mediated hyperacute rejection, nephritis, cytokine or chemokine induced lung injury, inflammatory bowel disease, Crohn's disease, or resulting from over production of cytokines (e.g., TNF or IL-1.) The antagonists may also be employed to treat one of the many conditions of the thymus, such as thymomas (neoplasm of thymic epithelial cells) and thymic follicular hyperplasia, as encountered in myasthenia gravis, Graves' disease, Addison's disease, SLE, scleroderma. and rheumatoid arthritis.

Instead of reducing arachidonic acid metabolism by inhibiting P450TEC expression at the nucleic acid level, activity of the P450TEC protein may be directly inhibited by binding to an agent, such as, for example, a suitable small molecule or drug. The present invention thus includes a method of screening drugs for their effect on activity (i.e. as a modulator, preferably an inhibitor) of a P450TEC protein. In particular, modulators of P450TEC activity, such as drugs or peptides, can be identified in a biological assay by expressing P450TEC in a cell, adding a substrate and detecting activity of P450TEC on the substrate in the presence or absence of a modulator. Thus, the P450TEC protein can be exposed to a prospective inhibitor or modulating drug and the effect on protein activity can be determined. For screening drugs for use in humans, P450TEC itself is particularly useful for testing the effectiveness of such drugs. Prospective drugs can also be tested for inhibition of the activity of other P450 cytochromes, which are desired not to be inhibited. In this way, drugs that selectively inhibit P450TEC over other P450s can be identified.

Polynucleotides that encode P450TEC protein or that encode proteins having a biological activity similar to that of a P450TEC protein, can be used to generate either transgenic animals or “knock-out” animals. These animals are useful in the development and screening of therapeutically useful reagents. A transgenic animal (e.g. a mouse) is an animal having cells that contain a transgene, which was introduced into the animal or an ancestor of the animal at a prenatal, e.g., an embryonic stage. A transgene is a DNA molecule that has integrated into the genome of a cell from which a transgenic animal develops.

In one embodiment, a human P450TEC cDNA, comprising the nucleotide sequence shown in SEQ ID NO:16, or an appropriate variant, fragment or subsequence thereof, can be used to generate transgenic animals that contain cells which express human P450TEC protein. Methods for generating transgenic animals, such as rats, hamsters, rabbits, sheep and pigs, and particularly mice, have become conventional in the art and are described, for example, in U.S. Pat. Nos. 4,736,866 and 4,870,009.

In a preferred embodiment, plasmids containing recombinant molecules of the present invention are microinjected into mouse embryos. In particular, the plasmids are microinjected into the male pronuclei of fertilized one-cell mouse embryos, the injected embryos at the 2-4 cell stage are transferred to pseudo-pregnant foster females, and the embryos in the foster females are allowed to develop to tern. (Hogan et al., A Laboratory Manual, Cold Spring Harbor, N.Y., Cold Spring Harbor Laboratory (1986).)

Alternatively, an embryonal stem cell line can be transfected with an expression vector comprising a polynucleotide encoding a protein having P450TEC activity, and cells containing the polynucleotide can be used to form aggregation chimeras with embryos from a suitable recipient mouse strain. The chimeric embryos can then be implanted into a suitable pseudo-pregnant female mouse of the appropriate strain and the embryo brought to term. Progeny harboring the transfected DNA in their germ cells can be used to breed uniformly transgenic mice.

Transgenic animals that include a copy of a P450TEC transgene introduced into the germ line of the animal at an embryonic stage can also be used to examine the effect of increased P450TEC expression in various tissues.

Conversely, “knock-out” animals that have a defective or altered P450TEC gene can be constructed. For example, with established techniques, a portion of the murine homolog of P450TEC DNA (e.g. an exon) can be deleted or replaced with another gene, such as a gene encoding a selectable marker, that can be used to monitor integration. The altered P450TEC DNA can then be transfected into an embryonal stem cell line where it will homologously recombine with the endogenous P450TEC gene in certain cells. Clones containing the altered gene can be selected. Cells containing the altered gene are injected into a blastocyst of an animal, such as a mouse, to form aggregation chimeras and chimeric embryos are implanted as described above for transgenic animals. Transmission of the altered gene into the germline of a resultant animal can be confirmed using standard techniques and the animal can be used to breed animals having an altered P450TEC gene in every cell (Lemoine and Cooper, Gene Therapy, Human Molecular Genetics Series, BIOS Scientific Publishers, Oxford, U.K. (1996)). Accordingly, a knockout animal can be made which cannot express a functional P450TEC protein. Such a knockout animal can be used, for example, to test the effectiveness of an agent in the absence of a P450TEC protein, if lack of P450TEC expression does not result in lethality.

Having generally described the invention, the same will be more readily understood by reference to the following examples, which are provided by way of illustration and are not intended as limiting. The examples are carried out using standard techniques, which are well known and routine to those of skill in the art, except where otherwise described in detail.

### EXAMPLES

### Example 1

### Isolation of the P450TEC cDNA Clone

The human expressed sequence tagged (EST) database at the National Center for Biotechnology Information (NCBI), available over the Internet at http://www.ncbi.nlm.nih.gov/BLAST/ was searched using an amino acid sequence encoding a typical heme binding motif found in all cytochrome P450s. The database was queried using the following sequence: PF(G/S)xGx(A/R/H)xCxGxx(F/L/I)A (SEQ ID NO:1). The TBLASTN algorithm of the Advanced BLAST program was used to search all 6 possible reading frames for translation of all the human EST sequences against the query sequence (SEQ ID NO:1). Parameters for all searching were the defaults of Blosum 62 which use a gap existence cost of 11, per residue cost of 1 and lambda ratio of 0.85. The EXPECT option, which is the statistical significance threshold for reporting matches against database sequences, was set at 1000 and the Advanced Option-E, which is the cost to extend a gap, was set at 10000. Subject amino acid sequences which showed similarity to SEQ ID NO:1 were retrieved from the GenBank database and their nucleotide sequences used to search GenBank for nucleotide sequences showing similarity to the EST nucleotide query sequence using the BLASTN algorithm to look for genomic or full length cDNAs.

One of the subject nucleotide sequence obtained from GenBank (AI216236, identified here as SEQ ID NO:2) also showed similarity to a genomic human DNA sequence from GenBank, Accession No. AC000016. In order to check for protein sequences that showed similarity to the 6 possible reading frames for translation of SEQ ID NO:2, the BLASTX program was run on the non redundant GenBank database. Several sequences with the highest degree of amino acid similarity were identified and included P450 18; Drosophila melanogaster, P450 16a; and mouse and human 2D6. None of the sequences had an identity value higher than 60%. EST clone A1216236 was obtained from Research Genetics, Alabama, and sequenced (Cortec DNA Service Laboratories, Inc., Kingston, ON) (SEQ ID NO:4).

A human thymus 5′ Stretch Plus cDNA library (Clontech, CA) was screened using a α-[³²P]-dATP labeled portion of EST clone AI216236 (SEQ ID NO:2), as per manufacturer's protocol for hybridization screening of ITriplEx libraries at high stringency. One clone, 2256 bp (SEQ ID NO:5) in length was isolated and sequenced (Cortec, ON). The clone was only 40 amino acids longer in the 5′ direction than SEQ ID NO:2. The clone extended 1750 bp further into the 3′ untranslated region. SMART™ RACE cDNA Amplification Kit (Clontech, CA) and thymus polyA+RNA (Ambion, Inc., TX) were used to amplify clones longer than SEQ ID NO:5. A primer within SEQ ID NO:5, 5′ AAGGACAGTGMTCCAGCMCT-3′ (SEQ ID NO:6), and the SMART II oligonucleotide, 5′-MGCAGTGGTAACMCGCAGAGTACGCGGG 3′ (SEQ ID NO:7) were used to prepare 5′-RACE-Ready cDNA as per manufacturer's directions (Clontech, CA). The 5′-RACE was also performed using the recommended procedure. Another primer from SEQ ID NO:5, 5′-GGTTTCTCCCAAATGGCTGGGTCTCT-3′ (SEQ ID NO:8) and the Smart Universal Primer Mix, Long 5° CTAATACGACTCACTATAGGGCAAGC AGTGGTAACAACCAGAT-3′ (SEQ ID NO:9) and Short 5′-CTAATACG ACTCACTATAGGGC-3′ (SEQ ID NO:10) were used to amplify cDNA clones from the 5′ RACE-Ready cDNA made from thymus RNA. The thermal cycling was performed in a PE GeneAmp 2400 System. The cycling conditions were:

1 minute at 94° C.; 5 cycles of (5 seconds at 94° C., 3 minutes at 72° C.); 5 cycles of (5 seconds at 94° C., 10 seconds at 70° C., 3 minutes at 72° C.); 35 cycles of (5 seconds at 94° C., 10 seconds at 68° C., 3 minutes at 72° C.); hold at 4° C. The reactions were run on a 1.2% agarose gel. Multiple products were observed from the cycling reactions. A nested PCR reaction was performed using a primer based on genomic sequence, 5′ of SEQ ID NO:5 but within the open reading frame, 5′-CCACCACAGTTAGCCTCTGCACTTCC-3′ (SEQ ID NO: 11) and the nested universal primer, 5′-AAGCAGTGGTAACAACGCAGAGT-3′ (SEQ ID NO:12) supplied in the SMART Kit. The thermocycling conditions were: 1 min at 94° C.; 35 cycles of (5 sec at 94° C., 10 sec at 68° C., 3 min at 72° C.); hold at 4° C. The reaction was run on a 1.2% agarose gel for analysis. One specific band was observed. The nested 5′-RACE PCR reaction was shotgun cloned into pTAdv vector using the AdvanTAge PCR Cloning Kit (Clontech, CA), as per manufacturer's directions. One clone was sequenced (Cortec, ON) and identified as SEQ ID NO:13. This clone contained about 880bp of sequence upstream of SEQ ID NO:8. This was a partial cDNA clone. There appeared to be a large GC rich region at the N terminus of the potential cytochrome P450.

Three primers, SEQ ID NO:11, SEQ ID NO:14 (5′ GAGGTCATATGAGGAATGGCAAGCG-3′), SEQ ID NO:15 (5′ TGCCCTTGGCTTTATTACCTTCCC-3′), and a plasmid containing cDNA from the N-terminal region (SEQ ID NO:13) isolated using the SMARTTm RACE cDNA Amplification Kit, were used to clone cDNAs from a human thymus cDNA library via homologous recombination technology. One of 5 clones contained a putative full length cytochrome, which we have named P450TEC (Thymus Expressed Cytochrome). This cDNA (SEQ ID NO:16) (FIG. 1A) is 3544 bp in length and contains an open reading frame encoding a putative protein of 544 amino acids (SEQ ID NO:17) (FIG. 1B).

### Example 2

### Isolation of P450TEC Genomic Clones

A BLASTN search of the High Throughput Genomic Sequence database at NCBI, available over the Internet at http://www.ncbi.nlm.nih.gov/BLAST/, with SEQ ID NO:16 identified the genomic BAC clone, B4P3 (GenBank Accession No. AC000016). The human clone is located on chromosome 4, mapped to 4q25 but is not completely sequenced. B4P3 sequence is in 9 unordered pieces and a portion of SEQ ID NO:16 aligns to contig 8 (SEQ ID NO:3), with distinct intron/exon boundaries. About 530 bp of the N-terminal region and nucleotides 751-1162 of the P450TEC cDNA are missing from the sequenced regions of the BAC clone. The genomic BAC clone, AC000016, was obtained from Research Genetics, Alabama, and digested with common restriction enzymes known to cut in distinct regions of SEQ ID NO:16. An α-[³²P]-dATP labeled probe containing a portion of SEQ ID NO:16 (nucleotides 1 to 222) was hybridized to the southern blot of the digestion products. The blot was hybridized for 2 hours, washed with 0.1×SSC, 0.5% SDS and exposed to X-ray film for 12 hours. An EcoR1 fragment of approximately 5000 bp showed a positive signal for the 5′ region of P450TEC. The EcoR1 fragments from duplicate samples were run on an agarose gel and the positive band was isolated and gel purified using the QIAEXII Gel Purification Kit (Qiagen, CA). The band was ligated into the pcDNA3.1(+) expression vector (Invitrogen, CA) and sequenced (Cortec, ON). The fragment contained 2038 bp of unknown genomic sequence for P450TEC (SEQ ID NO:55).

### Example 3

### Tissue Distribution of P450TEC

A 76 tissue human poly A+blot (Clontech, CA) was probed using an α-[³²P]-dATP labeled portion of SEQ ID NO:16 (nucleotides 1400 to 1779). The probe was randomly primed using the Prime-a-Gene Labeling System (Promega, Madison, Wis.). Hybridization conditions were as described in manufacturer's directions. FIG. 5 indicates that P450TEC appears to be expressed most abundantly in adult thymus and in fetal thymus. Lower level expression is evident in left and right cerebellum, corpus callosum, pituitary and aorta. It is possible that P450TEC is expressed in a variety of other tissues at even lower levels. A human northern blot (Clontech, CA) was also probed with the same labeled portion of SEQ ID NO:16 as above according to manufacturer's directions. FIG. 6 shows a distinct hybridizing transcript of at least 4.4 Kb, predominately in the thymus and possible low level expression in the heart and kidney.

### Example 4

### Metabolism of Arachidonic Acid by P450TEC

P450TEC is involved in the metabolism of arachidonic acid. To show that P450TEC protein metabolizes arachidonic acid and its derivatives or is regulated by these compounds, cells expressing P450TEC protein (either endogenously or after treatment with the appropriate chemical inducer or after transformation with an artificial DNA molecule expressing P450TEC, are used to prepare microsomal fractions. The microsomal fractions are used to assay the activity of the P450TEC protein against different compounds, including arachidonic acid, arachidonic acid metabolites and analogs.

A. Arachidonic Acid Metabolism and Product Characterization

The following general approach may be used to assess arachidonic acid metabolism by P450TEC protein and characterize the resultant products. Microsomal fractions are resuspended to a final reaction volume (0.2-0.5 ml) in 0.05 M Tris-Cl buffer (pH 7.5), containing 0.15 M KCI, 0.01 M MgCl2, 8mM sodium isocitrate, and 0.5 IU of isocitrate dehydrogenase/ml. Reactions are equilibrated at 37° C. with constant mixing for 2 min before the addition of [1 14C] arachidonic acid (25-55 μCi/μmol, 50-100 μM final concentration). Reactions are initiated by the addition of NADPH (1 mM final concentration) and continued at 37° C. with constant mixing. After 30-60 min, lipid-soluble products are extracted into ethyl ether, dried under a nitrogen stream, resolved by reverse phase HPLC, and quantified by on-line liquid scintillation using a Radiomatic Flo-One β-detector (Radiomatic Instruments, Tampa, Fla.) as described (Capdevila et al., Meth. Enzym. 187:385- 394 (1990)). Products are identified by comparing their reverse- and normal-phase HPLC properties with those of authentic standards and by gas chromatography/mass spectrometry (Capdevila et al., Meth. Enzym. 187:385-394 (1990); Clare et al., J. Chromatogr. 562:237-247 (1991); and Falck et al., J. Biol. Chem. 265:1024410249 (1990)). For rate determinations, the reactions are terminated after only 5-10 min to ensure that the quantitative assessment of the rates of product formation accurately reflect initial rates. For chiral analysis, the elcosatrienoic acids are collected batchwise from the HPLC eluent, derivatized to the corresponding EET-pentafluorobenzyl or EET-methyl esters, purified by normal-phase HPLC, resolved into the corresponding antipodes by chiral-phase HPLC, and quantified by liquid scintillation as described previously (Capdevila et al., Meth. Enzym. 187:385-394 (1990); Hammonds et al., Anal. Biochem. 182:300-303 (1989)). Microsomes not expressing P450TEC and reactions without the addition of NADPH are used as negative controls.

More specifically, the method used in the present invention is as described below.

Preparation of Microsomal Fractions

Cultured Sf9 insect cells expressing recombinant P450TEC were harvested 3 days after infection, washed twice with phosphate buffered saline (PBS) and resuspended in lysis buffer (100 mM Tris-HCl, pH 7.4, 1 mM EDTA, 0.5 M sucrose) containing protease inhibitor cocktail (Roche Diagnostics GmbH, Mannheim, Germany, 1 tablet per 10 ml lyses buffer). Cells were disrupted by brief sonication (5-8 bursts of 5 seconds duration) on ice using 550 Sonic Dismembrator (Fisher Scientific, Canada). The resulting cell lysates were successively centrifuged, at 800g, and 10,000g for 10 min each time at 4° C. Microsomal fractions from the 10,000g supernatants were pelleted by centrifugation at ˜1 00,000g for 60 min at 4° C. The pellet was then resuspended in microsomes storage buffer (10 mM Tris-HCl, pH 7.4, 5 mM MgCl2, 1mM EDTA, 150 mM KCI and 10% glycerol) and stored at −70° C.

Microsomal fractions were also prepared from uninfected Sf9 cells for control incubations.

Protein Determination

Protein concentration in microsomal fractions was measured by using BCA Protein Assay Reagent (Pierce, Rockford, Ill., USA) according to instructions provided by the manufacturer. Bovine serum albumin was used as standard.

Cytochrome P450 Determination

The cytochrome P450 (CYP) concentration of the microsomal fractions was determined spectrally by the method of Omura, T and Sato, R (1964) The carbon monoxide binding pigment of liver microsomes. Evidence for its hemoprotein nature. J. Biol. Chem. 239: 2370- 2378. Microsomal fractions were diluted to ˜1 mg/ml protein concentration in 100 mM potassium phosphate buffer, pH 7.4 and a baseline was obtained between 400-500 nm with spectrophotometer (Ultrospec 3000, Pharmacia Biotech). A few grains of solid sodium dithionite (1-2 mg) were added to the microsomal fractions.

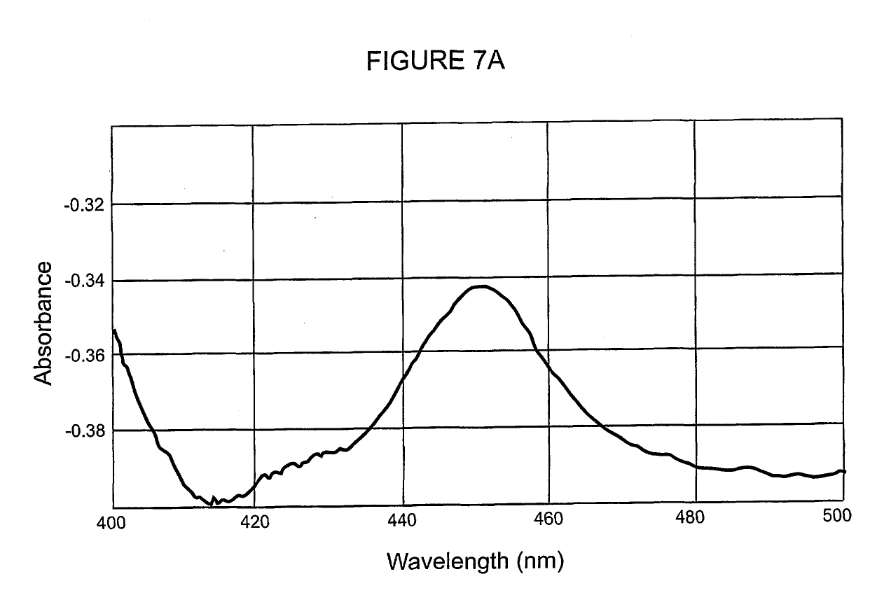

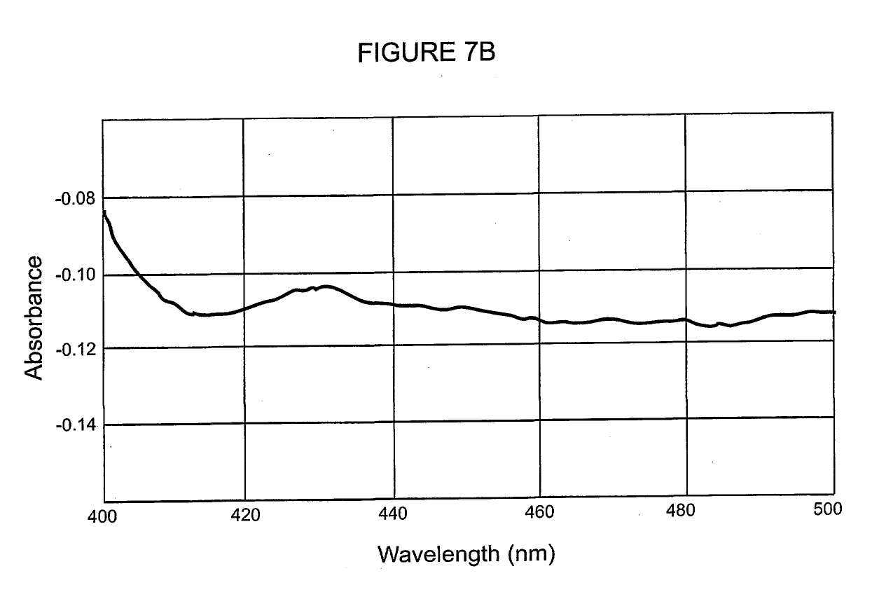

Following the addition of sodium dithionite, microsomal fractions were saturated with carbon monoxide and spectrum of reduced microsomes in the presence of carbon monoxide was recorded between 400-500 nm. The concentration of CYP was calculated from the absorbance difference between 450 nm and 490 nm using an extension coefficient of 91 mM⁻¹.cm⁻¹. FIG. 7A shows CO-reduced difference spectrum of CYP TEC expressed in baculovirus-Sf9 insect cell microsomes and FIG. 7B shows the CO-reduce difference spectrum of uninfected Sf9 insect cell microsomes.

Arachidonic Acid Metabolism

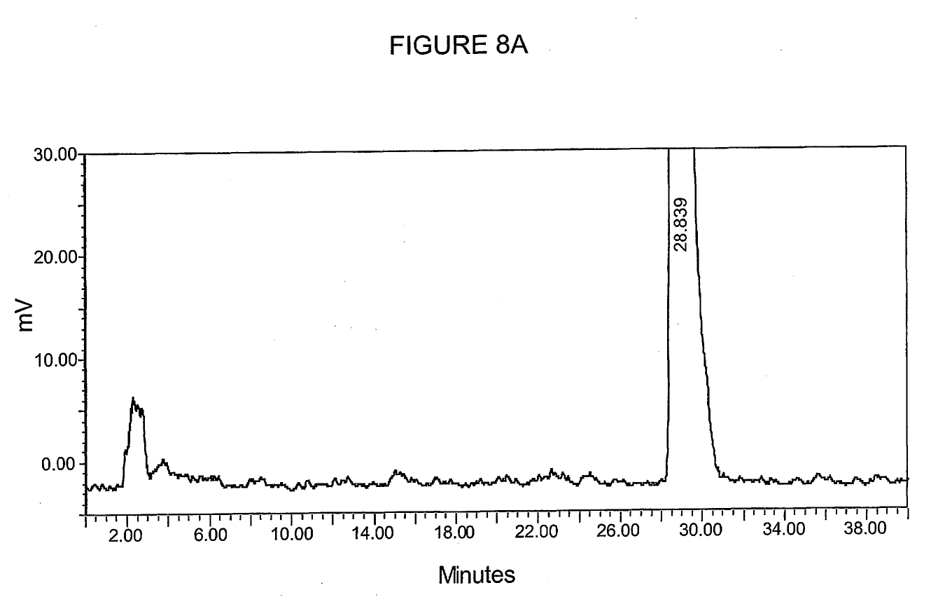

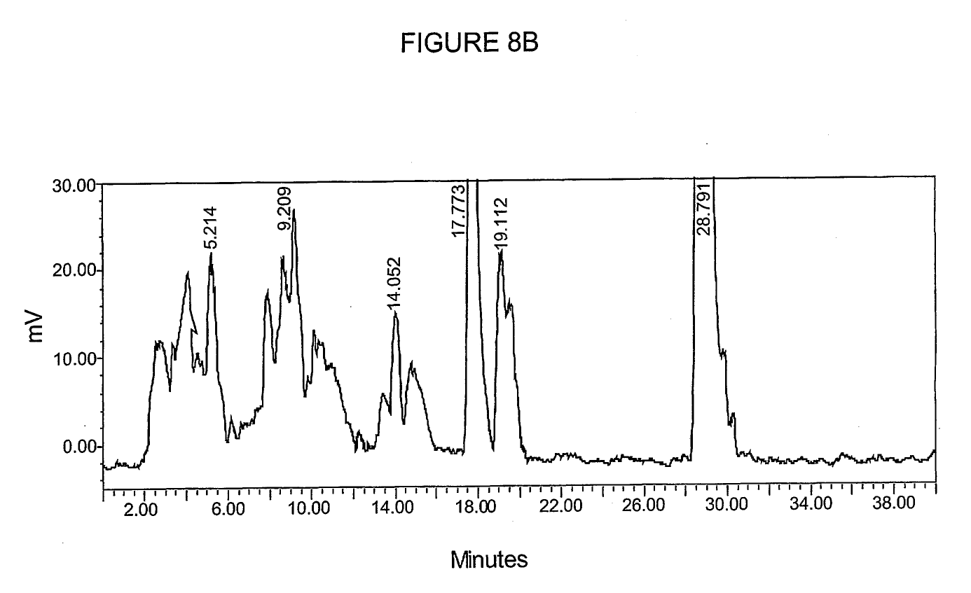

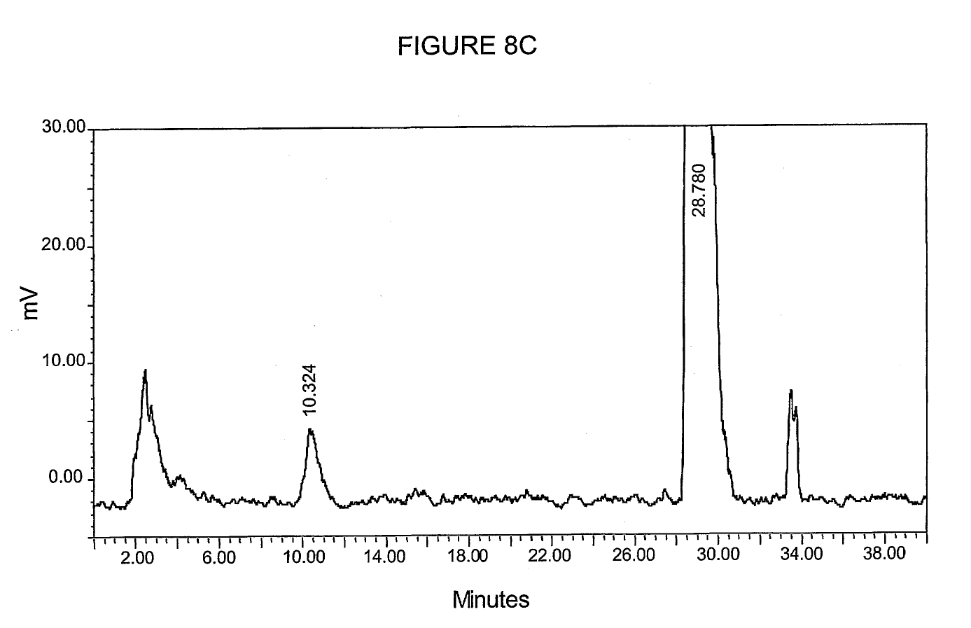

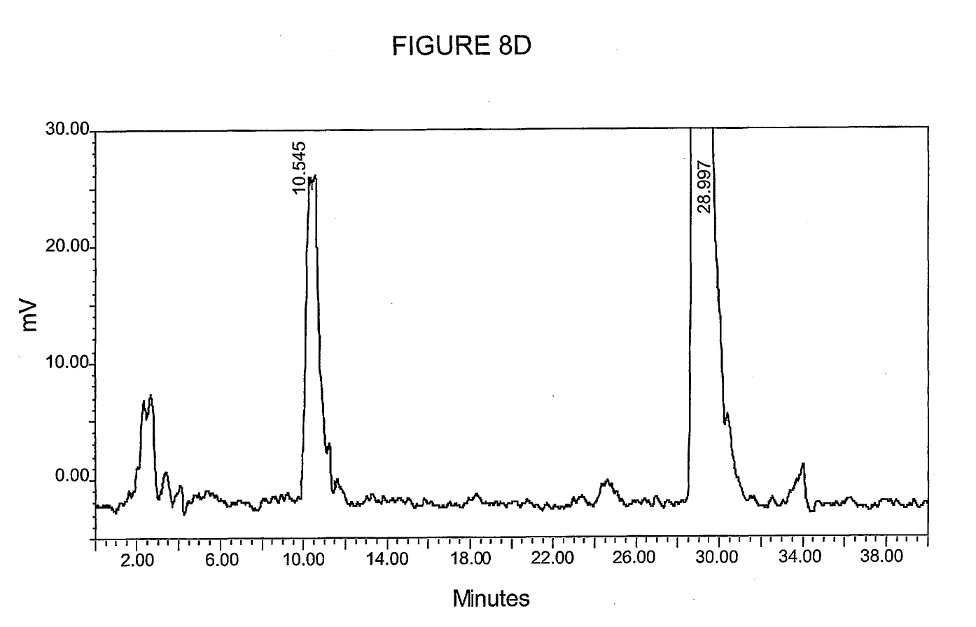

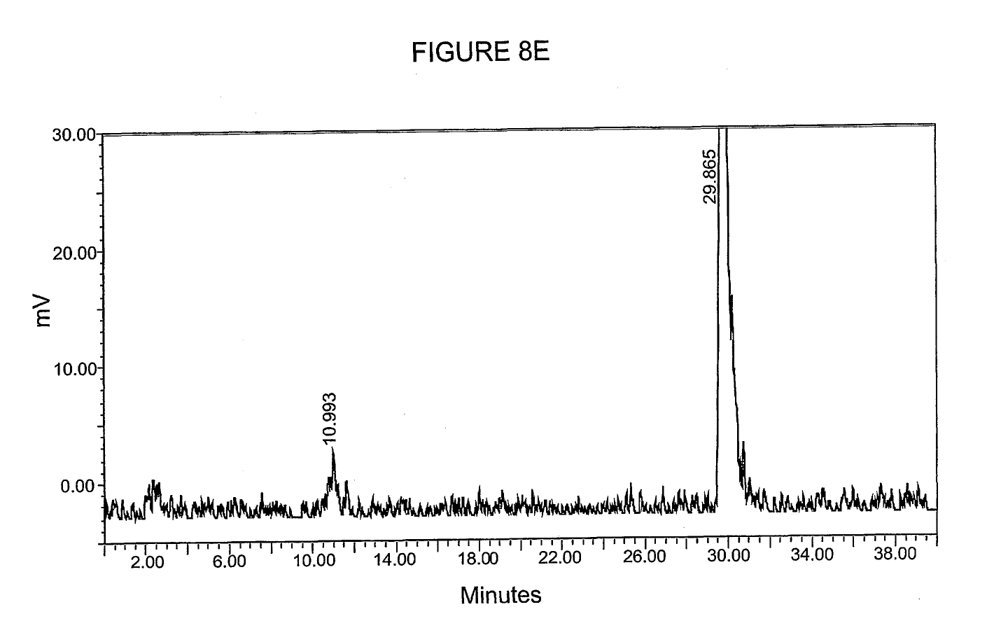

Microsomes (80 μg protein) were resuspended in 0.125 ml final reaction volume containing 20 μg human NADPH-CYP oxidoreductase+cytochrome b5 insect microsomes (Gentest, Woburn, Mass., USA), 100 mM potassium phosphate, pH 7.4, 3 mM MgCl₂, 1 mM EDTA, NADPH generating system (1 mM NADP+, 5 mM glucose- 6- phosphate and 1 units/ ml glucose-6-phosphate dehydrogenase) and 1, 5 or 20 μM [1-14C] arachidonic acid. The contents of reaction mixture without microsomes were equilibrated at 37° C. for 5 min. Reactions were initiated by the addition of microsomes and continued at 37° C. with gentle shaking (70 rpm/min). After 60 min, the reactions were terminated with 5 μl of 10% acetic acid and vigorous mixing. The incubation mixtures were extracted twice with 6 volumes of ethyl acetate, combined, evaporated to dryness under vacuum (Speed Vac^{R}, Savant Instrument, NY, USA) and resolubilized in 100% methanol containing 0.1% acetic acid for HPLC analysis (See FIGS. 8A-8E). FIG. 8C is the HPLC analysis with P450TEC microsomes in the presence of NADPH and 20 μM [1-14C] arachidonic acid without NADPH-CYP oxidoreductase+b5. FIGS. 8D and 8E are the HPLC analysis of P450TEC microsomes in the presence of NADPH-CYP oxidoreductase+b5 and NADPH and 20 μM [1-14C] arachidonic acid (FIG. 8D) and 1 μM [1-14C] arachidonic acid, respectively.

Reactions with P450TEC in the presence of 20 μM [1-14C] arachidonic acid but without the addition of NADPH-CYP oxidoreductase+cytochrome b5 or NADPH (See FIG. 8A) were used as a negative control. For comparison the profile of CYP2C9 mediated metabolism of [1-14C] arachidonic acid is shown in FIG. 8B.

One can see from the HPLC data that P450TEC is involved in arachidonic acid metabolism to the metabolite eluting at 10.323 to 10.993 minutes on FIGS. 8C to 8E, respectively. This metabolite represents a monohydroxylated arachidonic acid.

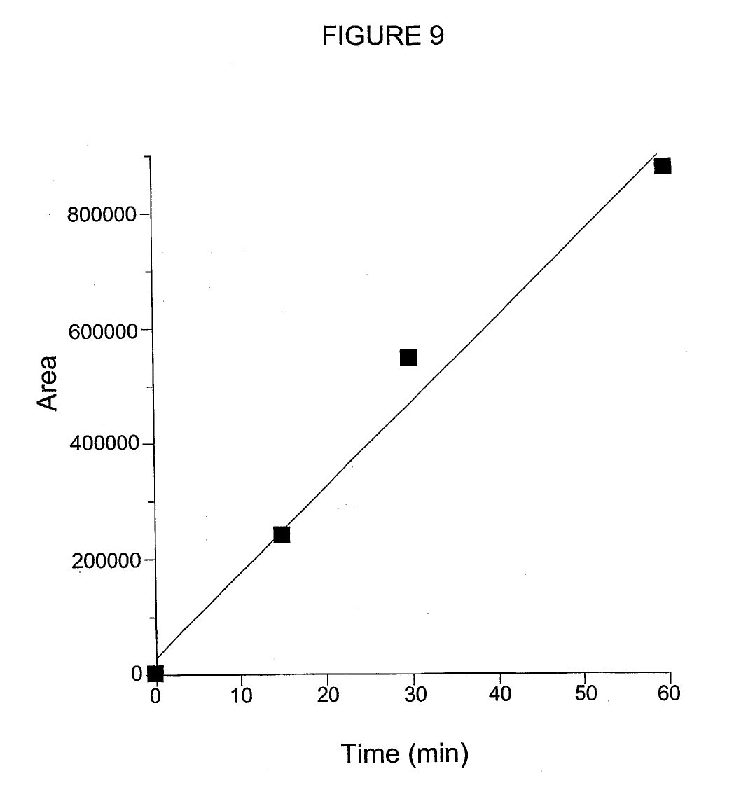

Reactions were also terminated after 15, 30 and 60 min incubation to determine linearity in arachidonic acid metabolite formation. (See FIG. 9). One can see from the graph that metabolite formation is directly porportional to time of incubation, for the time period and concentrations studied.

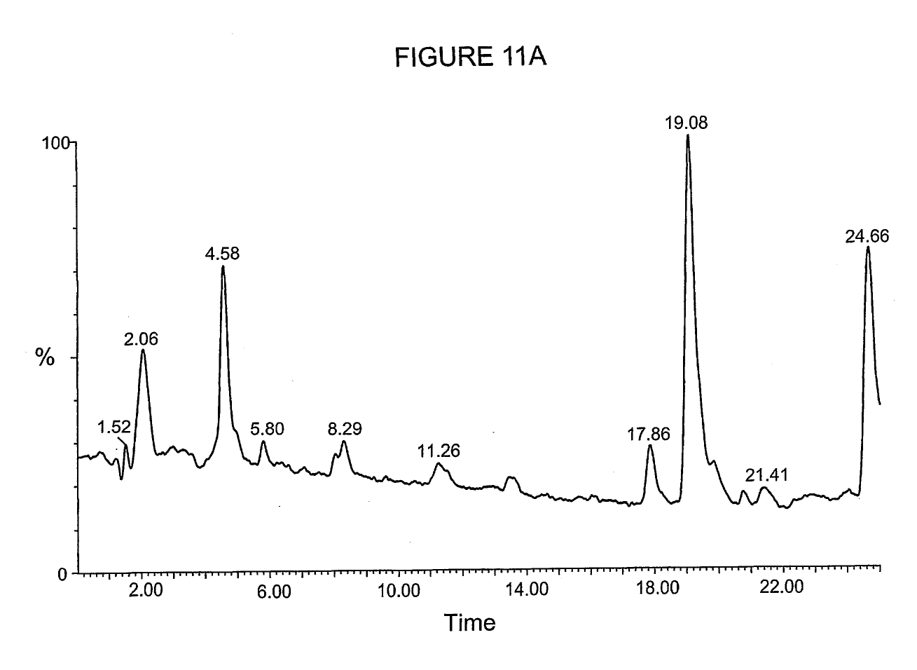

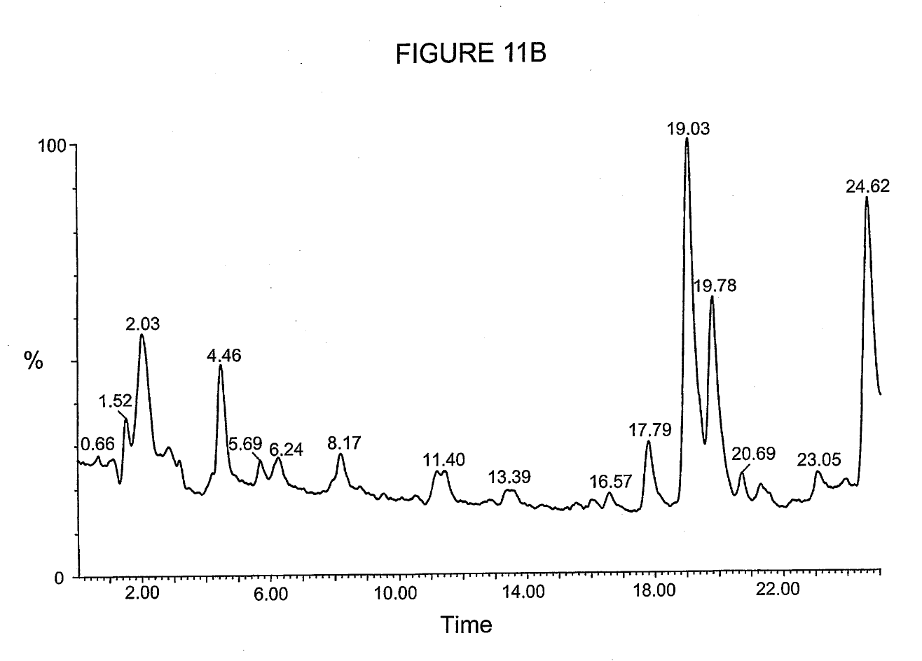

For MS and MS/MS analyses of arachidonic acid metabolites, 5 μM of unlabeled arachidonic acid was incubated for 60 min. The reaction was stopped by the addition of 5 μl 10% acetic acid. The reaction mixture was extracted with ethyl acetate and combined from 20 separate incubation mixtures, evaporated under vacuum and reconstituted in 100% methanol containing 0.1% acetic acid. See FIG. 11B for P450TEC microsomal incubations with arachidonic acid, NADPH-CYP oxidoreductase+b5 and NADPH. Similarly, ethyl acetate extracts from microsomal incubations without arachidonic acid was also processed and analyzed by LC-MS. (See FIG. 11A).

HPLC Analysis

HPLC analyses of samples were performed with a Waters Alliance 2690 Separations Module equipped with online degasser, an automatic sampling device, a 996-diode array detector (Waters corp., Milford, Mass., USA) and a radiometric detector (Radioflow Detector LB 509, EG&G Berthold, Bad wildbad, Germany). Arachidonic acid and its metabolites were resolved on a Zorbax Eclipse XDB-C18 column (4.6×150 mm, 5 μm; Agilent Technologies, USA) by using following gradient:

TABLE 1

| Time | Flow rate | | Acetonitrile | 10% Acetic acid |
|---|---|---|---|---|
| (min) | (ml/min) | Water (%) | (%) | (%) |
| 0 | 1.00 | 49.5 | 59.5 | 1.0 |
| 40 | 1.00 | 4.0 | 95.0 | 1.0 |
| 42 | 1.00 | 4.0 | 95.0 | 1.0 |
| 44 | 1.00 | 49.5 | 59.5 | 1.0 |
| 47 | 1.00 | 49.5 | 59.5 | 1.0 |

Millinium _{®}³² software (Waters corp. Milford, MA, USA) was used for HPLC operation and data processing.

LC-MS Analysis

The HPLC system for LC-MS was Waters Alliance 2690 Separations Module (Waters Corp. Milford, Mass., USA). The column used was Zorbax Eclipse XDB-C18 (4.6×150 mm, 5 μm; Agilent Technologies, USA) with a zero volume splitter. The following gradient was used for HPLC during MS analysis:

TABLE 2

| Time | Flow rate | | Acetonitrile | 10% Acetic acid |
|---|---|---|---|---|
| (min) | (ml/min) | Water (%) | (%) | (%) |
| 0 | 1.00 | 39.5 | 59.5 | 1.0 |
| 30 | 1.00 | 0.0 | 99.0 | 1.0 |
| 32 | 1.00 | 0.0 | 99.0 | 1.0 |
| 34 | 1.00 | 39.5 | 59.5 | 1.0 |
| 37 | 1.00 | 39.5 | 59.5 | 1.0 |

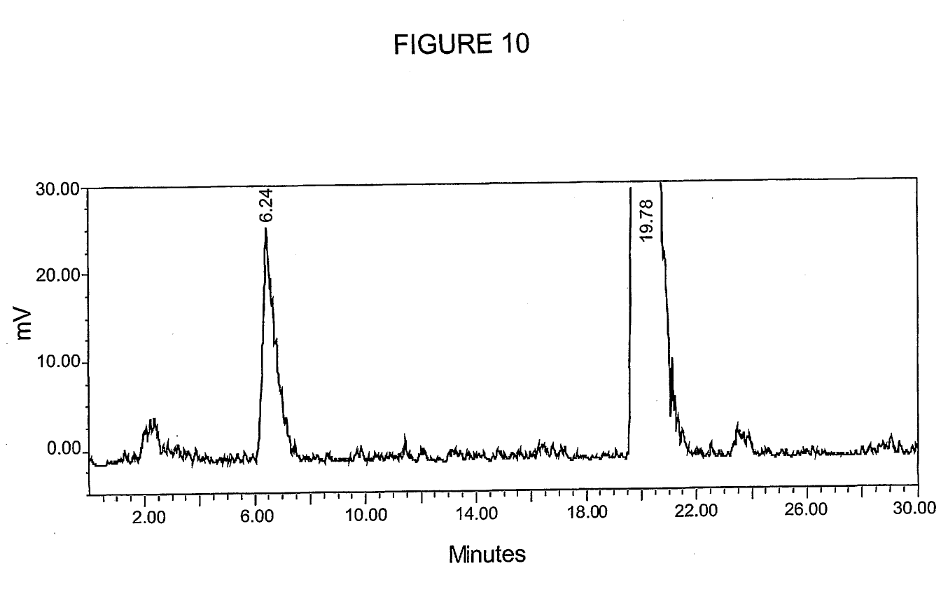

The HPLC elution profile showing arachidonic acid metabolism by P450TEC using the LC-MS solvent gradient is seen in FIG. 10.

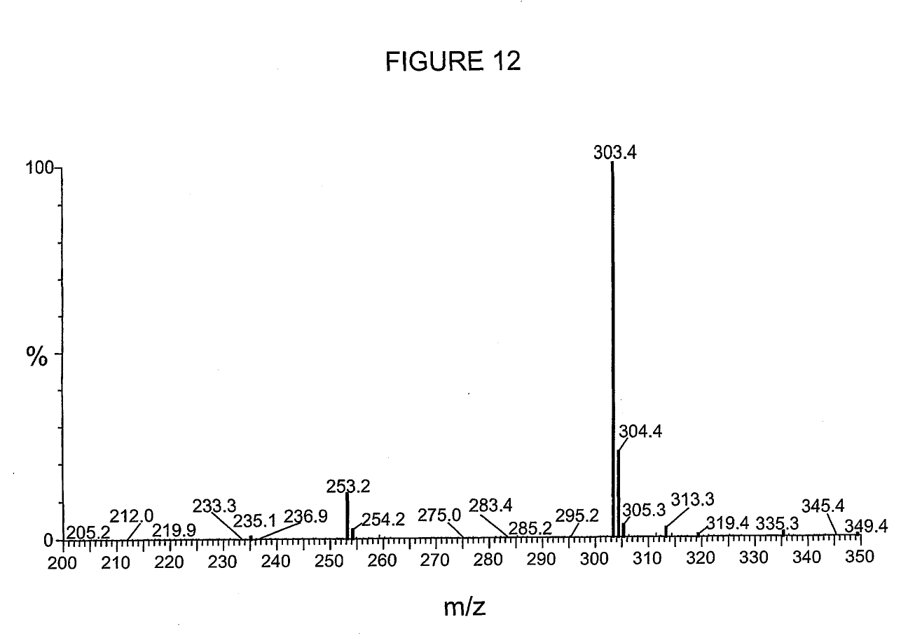

The effluent (10%, 0.1 ml/min) was connected to a mass spectrometer (Quattro Ultima, Micromass, UK) and subjected to ESI in negative ion mode. Arachidonic acid was used for tuning which was infused into the MS with a syringe pump at a rate of 5 pl/min. The following conditions were used under ES- mode for arachidonic acid and its metabolite(s): capillary voltage-3.00 kV, cone voltage-30 V, source temperature −85° C. for tuning and 130° C. for LC-MS, and desolvation temperature-140° C. for tuning and −300°C. for LC-MS. The MS spectra of the peak eluted at 19.78 minutes showed characteristics of arachidonic acid (m/z 303.4). (See FIG. 12).

Effluent at 5.70-6.50 min was collected after injection of ethyl acetate extract of P450TEC microsomes incubated with arachidonic acid and used for MS/MS analysis using syringe pump at a rate of 5 μL. min. Argon was used as a collision gas for MS/MS analysis.

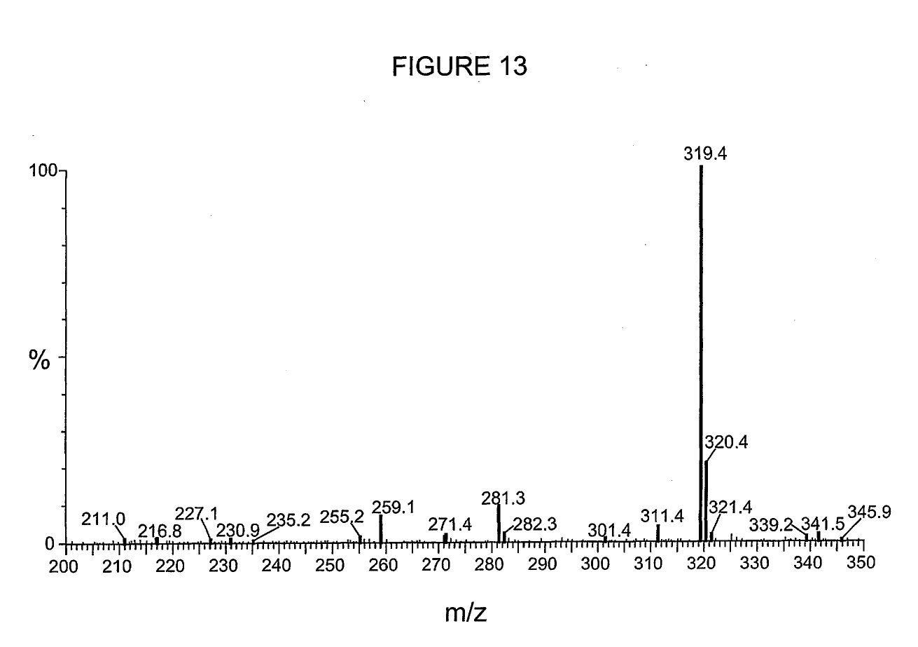

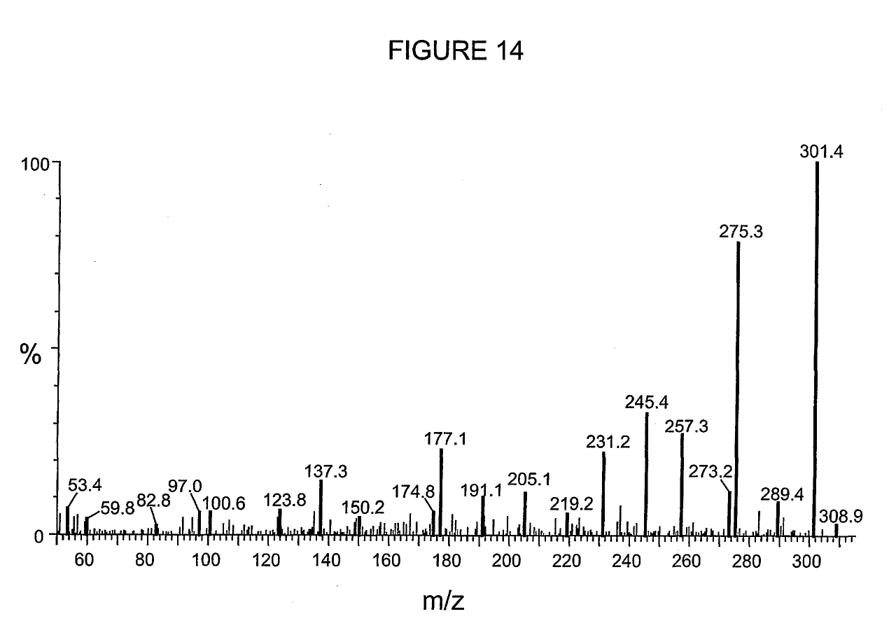

Mass Lynx 3.5 software (Micromass, UK) was used for LC-MS operation and data processing. FIG. 13 is the MS spectra of the peak eluted at 6.24 and FIG. 14 is the MS/MS of the peak from 6.24, respectively.

The data indicate that P450TEC is involved inarachidonic acid metabolism. Further, binding and enhancemnt by NADPH support microsomal localization of the enzyme.

### Example 5

### Protein Fusions of P450TEC

P450TEC polypeptides are preferably fused to other proteins. These fusion proteins can be used for a variety of applications. For example, fusion of P450TEC polypeptides to His-tag, HA-tag, protein A, IgG domains, and maltose binding protein facilitates purification. (see also EP A 394,827; Traunecker et al., Nature 331:84-86 (1988).) Similarly, fusion to IgG-1, IgG-3, and albumin increases the half-life time in vivo. Intracellular localization signals (e.g., golgi, nuclear, endoplasmic reticulum, mitochondrial) fused to P450TEC polypeptides can target the protein to a specific subcellular localization, while covalent heterodimer or homodimers can increase or decrease the activity of a fusion protein. Fusion proteins can also create chimeric molecules having more than one function. Finally, fusion proteins can increase solubility and/or stability of the fused protein compared to the non-fused protein. All of the types of fusion proteins described above can be made by modifying the following protocol, which outlines the fusion of a polypeptide to an IgG molecule.

Briefly, the human Fc portion of the IgG molecule can be PCR amplified, using primers that span the 5′ and 3′ ends of the sequence described below. These primers also should have convenient restriction enzyme sites that will facilitate cloning into an expression vector, preferably a mammalian expression vector.

For example, if pC4 (Accession No. 209646) is used, the human Fc portion can be ligated into the BamHI cloning site. Note that the 3′ BamHI site should be destroyed. Next, the vector containing the human Fc portion is re-restricted with BamHI to linearize the vector, and the P450TEC polynucleotide described in Example 1, is ligated into this BamHI site. Note that the polynucleotide is cloned without a stop codon, otherwise a fusion protein will not be produced.

If the naturally occurring signal sequence is used to produce the secreted protein, pC4 does not need a second signal peptide. Alternatively, if the naturally occurring signal sequence is not used, the vector can be modified to include a heterologous signal sequence. (See, e.g., WO 96/34891.)

Human IgG Fc region

| | |
|---|---|
| GGGATCCGGAGCCCAAATCTTCTGACAAAACTCACACATGCCCACCGTGCCCAGCAC | (SEQ ID NO:56) |
| CTGAATTCGAGGGTGCACCGTCAGTCTTCCTCTTCCCCCCAAAACCCAAGGACACCC | |
| TCATGATCTCCCGGACTCCTGAGGTCACATGCGTGGTGGTGGACGTAAGCCACGAAG | |
| ACCCTGAGGTCAAGTTCAACTGGTACGTGGACGGCGTGGAGGTGCATAATGCCAAGA | |
| CAAAGCCGCGGGAGGAGCAGTACAACAGCACGTACCGTGTGGTCAGCGTCCTCACCG | |
| TCCTGCACCAGGACTGGCTGAATGGCAAGGAGTACAAGTGCAAGGTCTCCAACAAAG | |
| CCCTCCCAACCCCCATCGAGAAAACCATCTCCAAAGCCAAAGGGCAGCCCCGAGAAC | |
| CACAGGTGTACACCCTGCCCCCATCCCGGGATGAGCTGACCAAGAACCAGGTCAGCC | |
| TGACCTGCCTGGTCAAAGGCTTCTATCCAAGCGACATCGCCGTGGAGTGGGAGAGCA | |
| ATGGGCAGCCGGAGAACAACTACAAGACCACGCCTCCCGTGCTGGACTCCGACGGCT | |
| CCTTCTTCCTCTACAGCAAGCTCACCGTGGACAAGAGCAGGTGGCAGCAGGGGAACG | |
| TCTTCTCATGCTCCGTGATGCATGAGGCTCTGCACAACCACTACACGCAGAAGAGCC | |
| TCTCCCTGTCTCCGGGTAAATGAGTGCGACGGCCGCGACTCTAGAGGAT. | |

### Example 6

### Construction of N-Terminal and/or C-Terminal Deletion Mutants

The following general approach may be used to clone a N-terminal or C-terminal deletion P450TEC deletion mutant. Generally, two oligonucleotide primers of about 15-25 nucleotides are derived from the desired 5′ and 3′ positions of a polynucleotide of SEQ ID NO:16. The 5′ and 3′ positions of the primers are determined based on the desired P450TEC polynucleotide fragment. If necessary, an initiation and stop codon are added to the 5′ and 3′ primers, respectively, to express the P450TEC polypeptide fragment encoded by the polynucleotide fragment. Preferred P450TEC polynucleotide fragments are those encoding the N-terminal and C-terminal deletion mutants disclosed above in the “Polynucleotide and Polypeptide Fragments” section of the Specification.

Additional nucleotides containing restriction sites to facilitate cloning of the P450TEC polynucleotide fragment in a desired vector may also be added to the 5′ and 3′ primer sequences. The P450TEC polynucleotide fragment is amplified from genomic DNA or from the deposited cDNA clone using the appropriate PCR oligonucleotide primers and conditions discussed herein or known in the art. The P450TEC polypeptide fragments encoded by the P450TEC polynucleotide fragments of the present invention may be expressed and purified in the same general manner as the full length polypeptides, although routine modifications may be necessary due to the differences in chemical and physical properties between a particular fragment and full length polypeptide.

As a means of exemplifying but not limiting the present invention, the polynucleotide encoding the P450TEC polypeptide fragment is amplified and cloned as follows: A 5′ primer is generated comprising a restriction enzyme site followed by an initiation codon in frame with the polynucleotide sequence encoding amino acids 130-139 of the P450TEC protein. A 3′ primer is generated comprising a restriction enzyme site followed by a sequence complementary to a stop codon and complementary to the region of P450TEC corresponding to codons 530-521.

The amplified polynucleotide fragment and the expression vector are digested with restriction enzymes which recognize the sites in the primers. The digested polynucleotides are then ligated together. The P450TEC polynucleotide fragment is inserted into the restricted expression vector, preferably in a manner which places the P450TEC polypeptide fragment coding region downstream from the promoter. The ligation mixture is transformed into competent E. coli cells using standard procedures and as described in the Examples herein. Plasmid DNA is isolated from resistant colonies and the identity of the cloned DNA confirmed by restriction analysis, PCR and DNA sequencing.

### Example 7

### Expression of P450TEC, P450TEC Antibody Generation and A Method to Detect P450TEC

Construction of Transfer Vectors

Open reading frame of human cytochrome P450 TEC cDNA was PCR-amplified using Taq polymerase (Qiagen) and primers introducing unique EcoRI and BamHI restriction sites on 5′ and 3′-end of PCR product, respectively. All PCR reactions were carried under conditions recommended by Qiagen with the exception of denaturation step which was conducted at 99° C. in each cycle.

PCR product was cloned into pCR-TOPO vector (Invitrogen), transformed into TOP10 E.coli strain and selected on LB plates containing ampicillin. Recombinant clones were identified by EcoRI restriction enzyme digestion of plasmid DNA purified from individual colonies. Integrity of P450 TEC coding region was confirmed by sequencing (Cortec).

EcoRI-BamHI fragment containing P450TEC coding region was then subcloned into EcoRI and BamHI digested pVL1392 transfer vector (BD PharMingen). After transformation into competent TOP10 E.coli cells, colonies were selected as above on ampicillin/LB plates. Recombinant pVLTEC clones were identified by digestion of plasmid DNA purified from individual colonies with EcoRI and BamHI.

6xHis tag was introduced by replacing DraIII-BamHI 3′-end region of pVLTEC with DraIII-XhoI fragment of pcDNATECHis plasmid (containing six histidine codons inserted in phase in front of the stop codon at the 3′-end of P450TEC open reading frame). BamHI and XhoI-generated ends of respective DNA fragments were filled-in with Klenow polymerase as described elsewhere. After ligation with T4 DNA ligase, products were transformed into TOP10 cells and selected as above. Recombinant pVLTECHis clones were identified by EcoRI and NotI digestion of plasmid DNA isolated from individual bacterial colonies.

Insect Cell culture, Co-transfection and Baculovirus Amplification

Sf9 cells were obtained from BD PharMingen and maintained in TNM-FH medium (PharMingen) at 27° C.

Recombinant baculoviruses were constructed by co-transfection of Sf9 cells with purified pVLTEC or pVLTEC-His transfer plasmids and linearized baculovirus DNA using BaculoGold kit (PharMinGen). After two additional cycles of amplification, large scale recombinant baculovirus stocks were titrated by end-point dilution in 96-well tissue culture plates seeded with Sf9 cells. Infected wells were identified visually after 2 weeks.

Amplified BacTEC and BacTEC-His viral stocks were stored at 4° C.

Expression of Recombinant P450TEC

For small scale expression of recombinant P450TEC or P450TEC His proteins, T75 tissue culture flasks containing 2×106 cells/ml in TNM-FH medium were infected with BacTEC or BacTEC-His baculoviruses at MOI≈10. Infected cells were collected 3 days post infection and stored at −20° C.

To express large amounts of recombinant P450 TEC protein, Sf9 cells grown in TNM-FH medium in roller boftles (1×106 cells/ml) were infected with recombinant baculovirus at MOI=2. Culture medium was supplemented with hemin chloride (final concentration 2 mg/ml), d-amino-levulinic acid (final concentration 100 mM) and ferric citrate (final concentration 100 mM). Cells were collected by centrifugation 3 days post infection and immediately used to prepare microsomal fraction.

Polyclonal anti-TEC Antibody

Rabbit polyclonal antibodies were raised against KLH-conjugated oligopeptide IKDHQESLDRENPQD corresponding to amino acids 302-316 of P450TEC protein (on SEQ ID NO. 17). Peptide synthesis and immunizations were conducted by Research Genetics. Reactivity of rabbit sera was then tested against E.coli expressed fragment of P450TEC protein (aminoacids 259-544). Only serum #99847 recognized bacterially expressed P450TEC fragment.

Identification of Recombinant P450TEC Proteins

Recombinant baculovirus-infected Sf9 cells collected from T75 flasks (see above) were lysed in 1 ml of SDS-loading buffer. 5ml of each lysate were fractionated on 10% SDS-PAGE and electrotransferred onto nitrocellulose filter as described elsewhere. Duplicate filters were blocked in 2% bovine serum albumin (BSA) in PBS containing 0.05% Tween 20 (PBST) for 1 hour. They were then probed for 1 hour with rabbit anti-TEC serum #99847 (see above) or anti-histidine tag murine monoclonal antibody (Qiagen). Both antibodies were diluted 1:10,000 in PBST containing 0.1% BSA. Both filters were then rinsed three times in water and washed with three changes of PBST. Secondary antibody solutions were then applied for 1 hour. Goat anti-rabbit IgG horseradish peroxidase (Amersham) conjugate or goat anti-murine IgG horseradish peroxidase conjugate (Pierce) (each at 1:20,000 dilution in PBST containing 0.1% BSA) were used, respectively. Filters were then washed again as above and developed in enhanced chemiluminescence reagent (Pierce). Reactive protein bands were then visualized by exposure to X-ray film (Kodak).

Detection of Recombinant P450TEC Proteins in Baculovirus-infected Insect Cells

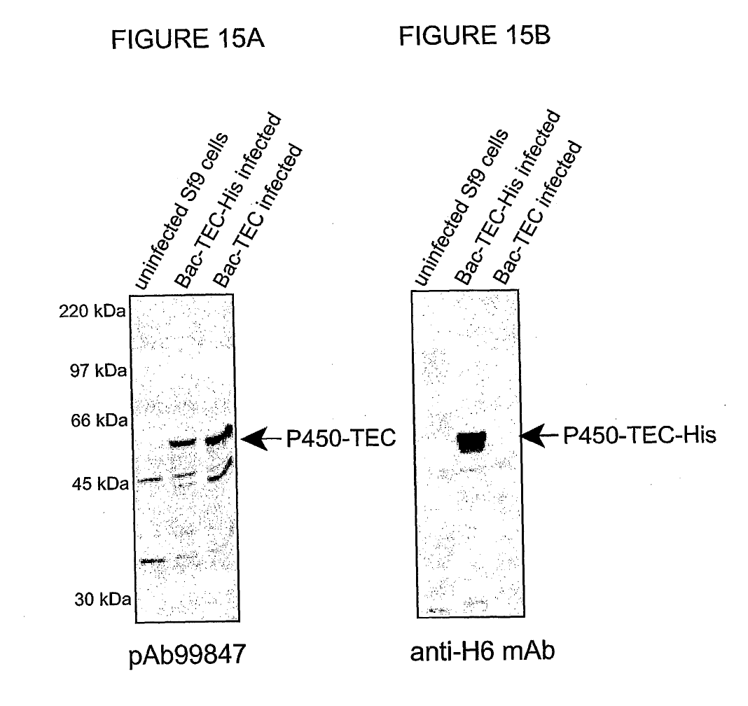

Total cell lysates of control Sf9 cells or cells infected with baculoviruses containing P450TEC or P450TEC-His recombinant gene were fractionated on 10% SDS-PAGE and immobilized onto nitrocellulose filter. Duplicate filters were probed with polyclonal antibody recognizing P450TEC-specific peptide (99847) (FIG. 15A) or monoclonal antibody binding to histidine tag (anti-H6) (FIG. 15B) and visualized with HRP-conjugated secondary antibodies and ECL. Positions of protein markers is indicated on the left (size listed in kDa). One can see from the filters that pAB99847 was capable of binding and detecting P450TEC.

### Example 8

### Formulating a Polypeptide

The P450TEC composition will be formulated and dosed in a fashion consistent with good medical practice, taking into account the clinical condition of the individual patient (especially the side effects of treatment with the P450TEC polypeptide alone), the site of delivery, the method of administration, the scheduling of administration, and other factors known to practitioners. The “effective amount” for purposes herein is thus determined by such considerations.

As a general proposition, the total pharmaceutically effective amount of P450TEC administered parenterally per dose will be in the range of about 1 μg/kg/day to 10 mg/kg/day of patient body weight, although, as noted above, this will be subject to therapeutic discretion. More preferably, this dose is at least 0.01 mg/kg/day, and most preferably for humans between about 0.01 and 1 mg/kg/day for the hormone. If given continuously, P450TEC is typically administered at a dose rate of about 1 μg/kg/hour to about 50 μg/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect.

Pharmaceutical compositions containing P450TEC are administered orally, rectally, parenterally, intracisternally, intravaginally, intraperitoneally, topically (as by powders, ointments, gels, drops or transdemal patch), bucally, or as an oral or nasal spray. “Pharmaceutically acceptable carrier” refers to a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion.

P450TEC is also suitably administered by sustained-release systems. Suitable examples of sustained-release compositions include semi-permeable polymer matrices in the form of shaped articles, e.g., films, or mirocapsules. Sustained-release matrices include polylactides (U.S. Pat. No. 3,773,919, EP 58,481), copolymers of L-glutamic acid and gamma-ethyl-L-glutamate (Sidman, U. et al., Biopolymers 22:547-556 (1983)), poly (2 hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al.) or poly-D-(-)-3 hydroxybutyric acid (EP 133,988). Sustained-release compositions also include liposomally entrapped P450TEC polypeptides. Liposomes containing the P450TEC are prepared by methods known per se: DE 3,218,12 1; Epstein et al., Proc. Natl. Acad. Sci. USA 82:3688-3692 (1985); Hwang et al., Proc. Natl. Acad. Sci. USA 77:4030-4034 (1980); EP 52,322; EP 36,676; EP 88,046; EP 143,949; EP 142,641; Japanese Pat. Appl. 83 118008; U.S. Pat. Nos. 4,485,045 and 4,544,545; and EP 102,324. Ordinarily, the liposomes are of the small (about 200 800 Angstroms) unilamellar type in which the lipid content is greater than about 30 mol. percent cholesterol, the selected proportion being adjusted for the optimal secreted polypeptide therapy.

For parenteral administration, in one embodiment, P450TEC is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to polypeptides.

Generally, the formulations are prepared by contacting P450TEC uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

P450TEC is typically formulated in such vehicles at a concentration of about 0.1 mg/ml to 100 mg/ml, preferably 1-10 mg/ml, at a pH of about 3 to 8. It will be understood that the use of certain of the foregoing excipients, carriers, or stabilizers will result in the formation of polypeptide salts.

P450TEC used for therapeutic administration can be sterile. Sterility is readily accomplished by filtration through sterile filtration membranes (e.g., 0.2 micron membranes). Therapeutic polypeptide compositions generally are placed into a container having a sterile access port, for example, an intravenous solution bag or vial having a stopper pierceable by a hypodermic injection needle.

P450TEC polypeptides ordinarily will be stored in unit or multi dose containers, for example, sealed ampoules or vials, as an aqueous solution or as a lyophilized formulation for reconstitution. As an example of a lyophilized formulation, 10-ml vials are filled with 5ml of sterile-filtered 1% (w/v) aqueous P450TEC polypeptide solution, and the resulting mixture is lyophilized. The infusion solution is prepared by reconstituting the lyophilized P450TEC polypeptide using bacteriostatic Water-for Injection.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, P450TEC may be employed in conjunction with other therapeutic compounds.

It will be clear that the invention may be practiced otherwise than as particularly described in the foregoing description and examples. Numerous modifications and variations of the present invention are possible in light of the above teachings and, therefore, within the scope of the appended claims, the invention may be practiced otherwise than as particularly described.

The entire disclosure of each document cited (including patents, patent applications, journal articles, abstracts, laboratory manuals, books, or other disclosures) in the Background of the Invention, Detailed Description, Examples, and Sequence Listing is hereby incorporated herein by reference.

What is claimed is:

1. An isolated nucleic acid molecule comprising a polynucleotide encoding a cytochrome P450 arachidonic acid metabolizing peptide or functional fragment thereof having a sequence selected from the group consisting of:
i. a polynucleotide encoding a polypeptide comprising amino acids from about 1 to 544 of SEQ ID NO. 17;
ii. a polynucleotide consisting of the nucleotide sequence of SEQ ID NO. 16;
iii. a polynucleotide consisting of the nucleotide sequence of residues 40-1672 of SEQ ID No. 16;
iv. a polynucleotide encoding the amino acid sequence encoded by the cDNA clone contained in ATCC Deposit No. PTA-1785;
v. a polynucleotide fragment of SEQ ID No. 16 or a polynucleotide fragment of the cDNA sequence contained in ATCC Deposit No. PTA-1785;
vi. a polynucleotide encoding a polypeptide domain of SEQ ID NO. 17 or the cDNA sequence contained in ATCC Deposit No. PTA-1785;
vii. a polynucleotide encoding a polypeptide of SEQ ID NO 17 or the cDNA sequence included in ATCC Deposit No. PTA-1785 having biological activity;
viii. a polynucleotide that differs from any of the nucleic acid molecules of (i) to (vii) due to the degeneracy of the genetic code;
where the isolated nucleic acid molecule is not SEQ ID Nos. 2, or 18-52.

2. A polynucleotide encoding a polypetpide epitope of SEQ ID NO 17 or the cDNA sequence contained in ATCC Deposit No. PTA-1785;

3. A polynucleotide that is a variant of the isolated nucleic acid molecule of claim 1;

4. A polynucleotide that is an allelic variant of the isolated nucleic acid molecule of claim 1;

5. A polynucleotide that encodes a species homologue of the isolated nucleic acid molecule of claim 1;

6. A polynucleotide that is complimentary to any of the polynucleotides of claims 1-5

7. A polynucleotide of any one of claims 1-6 wherein T can also be U.

8. An isolated nucleic acid molecule that is at least 95% identical to the isolated nucleic acid molecule of claim 1.

9. An isolated nucleic acid molecule comprising the isolated nucleic acid molecule of any of claim 1 that can modulate or encodes a peptide that modulates hydroxylation of arachidonic acid.

10. An isolated nucleic acid molecule comprising a fragment of the isolated nucleic acid molecule of claim 1 that can modulate or encodes a peptide that modulates arachidonic acid metabolism.

11. The isolated nucleic acid molecule of claim 10 wherein the arachidonic acid metabolism is hydroxylation.

12. An isolated nucleic acid molecule that Is capable of hybridizing under stringent conditions to a polynucleotide of claim 1, wherein said polynucleotide does not hybridize under stringent conditions to a nucleotide sequence of only A residues or of only T or U residues.

13. The isolated nucleic acid molecule of claim 12 that Is not SEQ ID Nos. 2, or 18-52.

14. The isolated nucleic acid molecule of claim 13 that is at least 15 bases.

15. An isolated nucleic acid molecule consisting of a nucleotide sequence that encodes an antigenic fragment or epitope of a polypeptide encoded by the nucleic acid sequence of claim 1.

16. An isolated nucleic acid molecule of consisting of nucleotides 1400-1779 of SEQ ID NO. 16.

17. The isolated nucleic acid molecule of claim 10, wherein the polynucleotide fragment comprises a nucleotide sequence encoding a mature form or a secreted protein.

18. The isolated nucleic acid molecule of claim 1, wherein the polynucleotide fragment comprises a nucleotide sequence encoding the polypeptide sequence identified as SEQ ID NO:17 or the coding sequence contained in ATCC Deposit No. PTA-1785.

19. The isolated nucleic acid molecule of claim 1, wherein the polynucleotide fragment comprises the entire nucleotide sequence of SEQ ID NO:16 or the cDNA sequence contained in ATCC Deposit No. PTA-1785.

20. The isolated nucleic acid molecule of claim 18, wherein the nucleotide sequence comprises sequential nucleotide deletions from either the C-terminus or the N-terminus.

21. The isolated nucleic acid molecule of claim 19, wherein the nucleotide sequence comprises sequential nucleotide deletions from either the C-terminus or the N-terminus.

22. A recombinant vector comprising the isolated nucleic acid molecule of anyone of claims 1-11.

23. A method of making a recombinant host cell comprising the isolated nucleic acid molecule of any one of claims 1-11.

24. A recombinant host cell produced by the method of claim 23.

25. The recombinant host cell of claim 24 comprising vector sequences.

26. An isolated cytochrome P450, arachachidonic acid metabolizing polypeptide or functional fragment thereof comprising an amino acid sequence selected from the group consisting of
(a) a polypeptide fragment of SEQ ID NO:17 or the encoded sequence included in ATCC Deposit No. PTA-1785;
(b) a polypeptide fragment of SEQ ID NO:17 or the encoded sequence included in ATCC Deposit No. PTA-1785 having biological activity;
(c) a polypeptide domain of SEQ ID NO:17 or the encoded sequence included in ATCC Deposit No. PTA-1785;
(d) a polypeptide epitope of SEQ ID NO:7 or the encoded sequence included in ATCC Deposit No. PTA-1785;
(e) a mature form of a secreted protein;
(f) a full length secreted protein;
(g) a variant of SEQ ID NO:17;
(h) an allelic variant of SEQ ID NO:17; or
(i) a species homologue of SEQ ID NO:17.

27. An isolated polypeptide that is at least 95% identical to the sequence of claim 26.

28. The isolated polypeptide of claim 27, wherein the mature form or the full length secreted protein comprises sequential amino acid deletions from either the C-terminus or the N terminus.

29. An isolated polypeptide of claim 28 or fragment thereof that has arachidonic acid hydroxylase activity.

30. The isolated polypeptide of claim 29 or fragment thereof wherein the arachidonic acid metabolizing activity is hydroxylase activity.

31. An isolated antibody that binds specifically to the isolated polypeptide of claim 26.

32. A recombinant host cell that expresses the isolated polypeptide of claim 26.

33. A method of making an isolated polypeptide comprising:
(a) culturing the recombinant host cell of claim 32 under conditions such that said polypeptide is expressed; and
(b) recovering said polypeptide.

34. The polypeptide produced by claim 33.

35. The gene corresponding to the cDNA sequence of SEQ ID NO:16.

36. A pharmaceutical composition comprising the isolated polypeptide of anyone of claims 26-30, and/or optionally a modulator of P450TEC activity in combination with a pharmaceutically acceptable carrier.

37. The pharmaceutical composition of claim 36 further comprising an adjuvant.

38. A method for preventing, treating or ameliorating a medical condition related to P450 TEC expression which comprises administering to a mammalian subject a therapeutically effective amount of the polypeptide of claim 26 or of the polynucleotide of claim 1 and/or a modulator of P450 TEC.

39. A method of identifying an activity in a biological assay, wherein the method comprises:
(a) expressing P450TEC in a cell;
(b) isolating the biological fraction;
(c) detecting an activity in a biological assay; and
(d) identifying the protein in the supernatant having the activity.

40. The product produced by the method of claim 39.

41. A method of diagnosis of a P450 TEC-related disease or condition or a predisposition to a P450 TEC-related disease or condition comprising detecting a polymorphism in a P450 TEC gene, wherein detection of said polymorphism is indicative of the occurrence of said disease, condition or a predisposition thereto.

42. A diagnostic kit for identification of polymorphisms in the P450 TEC gene, comprising screening the P450 TEC gene from a human for polymorphisms, wherein detection of said polymorphisms is indicative of the occurrence of a P450 TEC-related disease, condition or a predisposition thereto.

43. The method of claim 42 wherein the disease is related to arachidonic acid metabolism.

44. The method of claim 43 wherein the disease is related to hydroxylation pathway of arachidonic acid.

45. The method of claim 41 wherein disease related to P450TEC activity is an autoimmune disease.

46. The method of claim 41 wherein the disease related to P450TEC activity relates to the inflammatory response of a patient.

47. A use of a polypeptide of claim 26 and/or modulator thereof for treating a disease or condition related to. P450 TEC activity in a patient comprising administering to the patient in need thereof, a therapeutically effective amount of said polypeptide and/or agonist or antagonist thereof.

48. The use of claim 47 wherein the modulator is anagonist or antagonist of P450TEC.

49. The use according to claim 47 wherein the polypeptide consists of the amino acid sequence of SEQ ID NO. 17 or a biologically active fragment or analog thereof.

50. The use of claim 48 wherein an antagonist is an antibody to P450TEC.

51. A method of identifying modulators of P450TEC activity in a biological assay, wherein the method comprises:
(a) expressing P450TEC in a cell:
(b) adding a substrate; and
(c) detecting activity of P450TEC on said substrate in the presence or absence of a modulator.

52. The method of claim 51 wherein the substrate is arachidonic acid.

53. A method of quantifying the levels of P450TEC in a sample comprising:
(a) subjecting a sample wherein levels of P450TEC are to be quantified with a labelled P450TEC antibody;
(b) detecting all labelled antibody bound to P450TEC to determine P450TEC levels.


SEQUENCE LISTING (56 sequences)

SEQ ID NO: 1 - protein, 15 aa, Artificial Sequence
  Description of Artificial Sequence query sequence
PFXXGXXXCXGXXXA

SEQ ID NO: 2 - DNA, 363 bp, Homo sapiens
  unsure (33): n can be any nucleic acid
ccctaatcgatttctggatgaccaaggacaacnaattaaaaaagaaacctgtattccttttgggatagggaagcgggtgtgtctgggagaacaactggcaaagatggaatnattcctaacgtttgtgagcctaatgcagagtttggcatctgctcgcacctgagggttctaagaagcccctcctgactggaagatttggtctaactttagccccacatccatttaatataactatttcgaggagatgaacgccatctccaagaagagatggtaaaaagatatataaatacatatccttctaagcagattcttcctactgcaaaggacagtgaatccagcaactcagtggatccaagctgggct

SEQ ID NO: 3 - DNA, 43680 bp, Homo sapiens
  unsure (1)..(13): n can be any nucleic acid
nnnnnnnnnnnnngtggtgttgactctgtaaaatgctatcctttgatgacatgtgcatatctaggctctgaagaaatatgtgctgcttggctgtgttggtaatggacccaaaatgtgcaccgttggttgatgtcaccaaataatcaactcgggattattttaatcccgtggacagtgattgtttcccctaaaagggaaataaatgtagactgcgaacacccaaaacaaatgtctagtgaccatgagttacaatgcaaaaatcaggctcagggaaggtaaagagagagttgacctttagttacccatcttgacgttggacgtctatcagtgcagtgaacatgtggacggtttaaggtcatagagcaacctttctggaataacgaatactattagaaagcataggaaagaaatgtcttgtcaaataatggaagactggatagcagcttggccaatcttctcactcaggtgaatactttctaattactgtaacttagtgatataggattcttattaatttatttgctttatgtttaaatattgttaacaaaaaaacttcaaacaagtttagcagagtttatctgagcaaagaaagcatgaatcatgtagtatcctgagccagtaaaggttcagagagctccagcccctaacagtgggcaagccgcgtttatagacaatattttttgcagcctgtaatgggctaaaagctccgctgcggtgattggccaagacttggccacttgttagaagaacgtactctcaaattacattgcagtttatttacatattaggtttgattatgctttgctaggtagggaggcaactttaggccagatttaatttaataacagtttcctcctttgggtcagcctctcagttttacgagattgaccagaaccttgttttgtctcagtatggaagtcacaatgtcaaattagttgagtgattatactttcttcatgttgttattctaactgtaataacaccatttggtgtacaaaggatggctgcatagagacattgaagactctcgaggggacaacaacacatcagggagactattatggccacgagaaggagtatagcaataaactgaggtacacaccttaacaggagtctccacaaactgaactgatcaaaattaaacaattcaaagttcaggcaataaagatagttcaaagtatttgagtccaattggtcatggtctaatttagggcattatggcggttaaggactgctagaaatgcttatttcttggtgccgtaaagaaatagcacttgaacataaaattaatttctttagcaaggccatttttactttctgcggaaagagtacactcgccagcagttttgccacaagagtacaccgaacaaaggaggcagggtcatttataacctgacccgtccaccttactgttgtgactggtttccattggctagaacgggacttcacattctgtatttgtcttgattggctagcaacttagaaattcttaaaagaggcaaaggcagaggagaacaaaggaaggaggaagtaacttgtggaatgctgagaagggaaaaacacctctaaataaggaagaggaacaggctatgacctaatgcccacttggaccagtataagcatgccagggcaaatatttaggctaaattgtgggagctaagaacataaagtacattgatttctttattacagctagcagatatttaagaatgttaacaggtctttgaataaattttgcttctaagagaggttactatttattcctaatcagatgaggaggaaagtctttgaaaaggaacctctactttactttttacaaggaccatagttcactgaatgacctgattcagccttatggcctgatttaaagaggtatccatttttgtaattagcctggtaacacaagttataataacctggagagccactaaagaagcataaagattagaaaagtttggaatagcctagcttgcctttcactactcaggatgcctacaaaccaaccattagctgctcccataaatgtatcatgtgttcctttcccctgagaagtttccttaatatattcagtggcagtgtttagagaaacagcagtatctgccaccttttaaattaagttttctatagtagtaaaattagaggaaaaataagtgcaacattcagttttattcagcaccatgcaagagcccccagcatgagcaaagaggagatccaaagttgtgtaatgttccatgaagtgtgtttgttgaacacaaatgttttgatccactgagaatatttgagtagcttctaccatgtgaacccctaggaggtctgactggctacaaattcaagattttccccaatttatagattagtttcaaattccatacaacaggcaccctgctaatgccaagagtgagttccagcacctccattggaacctttcctcagtagaaactaacttgtcttcatttatttttaggttggtgcttactttggttattgactgctttaacctctgattatgaaaggtattatgggaactttcagggaaggctattgggatgtaggagcgagccaggccaaatagcaagatctgaaccagtgaggaaacaacagagaaggcaggtcagattatccaccaacccaatgtaggcccttgggggcttgaaaagagggccacttagtggtgtttgagccgaggtcagttaaatttgtccacccacaagttggcatagctcctgaataaaatccagtggtgaacttactcttctgtggtccctatatagcatgttgtaagggtgcaaaccactggatctagtaaaaagactttgctagatttaatccagtgaaatcacacaagcaattatttgtacccacataggtagtccccaacgtcttaagtatgcaatgcccggaagcacaagataccttttgcaggcatcattctgatagttttttttttttacttttatgtacaattgacaaataataattgtacatatttatggggtacagtgtgatgtctcaatgcatgtatatgttgaacaatgatcaaatcaggataattattaaataatatgtcactttaagcatttatcatttctttttggggataacattcaaaatcatctcttctagctatcttgaaatatacaatacattattattagctgtagtcattctactgtgtaatagaatagcagcttttattcttcctgtctaactgtaactttgcacccattgaccaatcccctctatccagattttttatatttggtaacagtttggttattaactggaaagatcaagatactattttcagcaagtataggaaggcaggtagcagtgatgtgtagcatatcaaagataaccttcctctctttccttggggttgcagggtgactctcattaggaacacaggcattagcgttaatgaaattgtttccagatttttgcgcattagctcataaacccaacagttactctggtttcttggagaccataagactttgccaaagccatccacagagtatcatcccaaattgacaaggaaattcagttatttctgttgcatacaacattttgagataacaactagaattacgattaatagccttataccaggactattagatttctattaatttcttacaagttctgaaatacatattaataacatatccttacatatataattcaaaaaagtctggcattatttagggttttcctaaagaaagggaaggaattagtattgcaggaaatagagaaaaaaggaaaaaaagagaaggctttcatgatagcaaagaactcttgatctgcaatattaggaaagctgtctacatataggatgccatatgcttctagggaaaaacttccctgatcagctttatttcaaggtctccaacaagtttatagttccagaagtctacaggacctcttttgtgttgagaaatgcagatccaagattcaaggccttgaagtttgctgcagagaagaacttggtatggtccctttcaaccgagttcaagtaatgtctttctctggagttatttccaaaagacccccacctccaggttctatattatgaaacacttggttgtcatcagttggtgggtcatgaaggacttctttcacttggtaaaaatatgctttggcagaatgcattcaaggcttgcactattaagtcatgtcagggtttataagaggggaagatacatgagactttattattaggggcataagccttccagtaactattttatgaggggtcaatttatgttttacagtgggaatggatcttattgccatcaatcagtaatacctttgaccaaggcaatccaatcaattcagttagcttttcctaagctattgtgtttataatacctgtttaactgttttacagcgtgtccagtgaaatgcatacctctctgttgctagaaatttctccggcaatgccccataagggaaacatattttccaataaccttttagctattgttatatcattggccttcctgcattggaaatcttctatacaaccagaaaacatgcatttaaagtggaaattgaatgaaagccatttgtaggtgttcaggaaatgtatcacctgaagtttttattgtcttaccacaattatgggtttgacaaaccagacattggttataaactattttagcaattttataacagtcaccccaccaatcttttttcataatttgaatcattttatctcctccataatgagtcatggagtgcagagcttttaatattggaagatttaagactccagaagaaccaggcagccatccaggttcttcatgcatccaggcttaacattggatttaattctcttaaataccaactttgtttccccaattcaggtgcttatagcactatttattgaataggttttatcaaatgtaatttgacttggatcaatattatatagttcattcaagttgtatatcttaatttcagtactggcttaattagcatgaaattctgcccaagtcatttcctttgtattatttttattctgtgtgacattaggttagcagttttatgaatcagtcaatttcttcattagagtctgggaattcttacccaacccaatggtgtgattttaaagttatgagaaacctgtaattctcagagttctttccataaatctccttgaagatgtacactttaggcttatggtttctcagaaagtatcagagtaaacaattaactatctgtgaatgacaagacttaaaatggccatgactttaaagatctgggaagagttcattacaataatgacacaattgacaaggaaactcagttacttctgttgtattcaacattttaagataattactagaattatgactgatagctttatagcagaactattagatttctatgaattttacacaatttctgaaatacatattaacacattctatacaaatataattcaaaaaaagtagtattatttattattttataatcctttccatataatttaatatatcagatcagtccaattactttaatatgtctctttctataacaagagatatccttttgagatattctgaaggtgcatatggaaaatctcaaagttaattcaatttcaagaaaagacttaatttagaattttattttggaaagtttataaaaaatatcaaaggtttaaaacatttgatcaaaaaaggatcacaagtcactgtgaacaataatcattcatttaatcagagtggtaatttaaagacttcaaaggcaaatacagagagttgcagagttgtaaattacattttttaataaagcggactcagttttcataagtgatcaaaaacccaataaaggcaacatgaagcacaggaaattatcttggtaaaatacagaatctttgcttcctaggccaattatataaaaagtaaagaaaaacttttcataatttcctatcaagagcaggccaacaattcaagaaaaccttattgttttaacagagaggactacattctagttttgcatcagtgtacttttgatattaatgctcaacttttggagaaactcaaataattttcttctaatattagccagcttaatcacacataaaactccatttataagattcatcttccagaaaacttctacaacttccttatctgttcagtttttgtcctgtacttttcctcttcacattttggaacaaccagtcattctatttgaggacaaaagttactctttttttcccttaacaaaaaaattctcataccttataactttttcttaaccagagcacatcttaacttcctgatacacttgcatatagagtttttctctatttctaataattctatatattaaataaaatatattaattagaatttttttttaatttgagatggagtctcgctctgtcgcccaggcttaagtgcagtggtgcaatctcggatcactgcaacctctgcctcccaggttcaagtgattctcctgcctcaggctccagagtagctgggactacaggcatgtgccaccatgcccagctaatttttgtatttttagtacagatgaggtgtcaccatattggccaggctggtctcgaactcctgacctcatgatctacctgccttagcctcccaaagtgctgggattacaggcgtgagccaccatgcccagccacaattagaattcttaatccttagtgaccttaactgccactgaacactacaaagcaagcaattgtgaactgtcacatcacccatggaactgccacatcactcatctgtaccttggcaaatccatcaatcattacttctagaagcatatgctttcttatagtacaattcatcagtgtcgcctagaacatgtttgtaacaattacaaatatctttagtgtatcagtaataagaagctgtagaggagacaaaatttcatctctagcctcctaggttttctttttttaatgctaggcctaagaattaaattgtggtttgtacagatttccctcaagttcagttttccatcttgataagaatgttaagaccttctaatataggaaggactgttttacatggaaattttatcttacatctgtttaactcacttatttttaacaattatgcatagattgtttataaagatgagacagtaagcagctagtcatcatctaagttatttctcttgctgacatattttgcaatatagaggtaacataagtttattttaccagtgaacctaggtaggaaaagttatgtgcctgtattatatttaatgctgaaaattctgaagacatgactgttttaatgaaatcaacaagattcttatttaccaagatcacccaagtaatgtgaatttgaaaagcattttagttcttaattttctgagtgaatacttcatttatattagcacttatttttgaaaaacccaaataaatagagcactttttcaattcaattttggcaatatcataaggagatacaaaaatatcacacatgtgtcatacatacagatctgtccataaacgtataagcagatacagatcttctatagctttcatttaaaatttttagccatttgccaggtgcacaataatataaaactcagtaatttattaaaaaatatctgaaccagaattgttttcctggccaatagaataagatttcctgcccccatgggtaaagcttttaaatagtatttgtgaaaaagatatttaagatttttttcatttgttttctgtaagcaaccttataaagaggcaatctttgctttatttgctgctttagatgcctgtacataccaatttaaaaatatatcccattgctttgtggcattttggattctttttttaatgcccctgcaaatgaacaattttatgtatgttaaaatttccccattgtaacaactataattctaagttgtcctagtaagggacatttttttcaaaaactcacaggttctaggcccataatttttgtgcataaagttagctggcataccaaaaagtggagtcccagattcttggaatcccagacttttctttttcttttcttttcttttttttttttttttgagggagttttgcactgtcacctaggctgaagtgtagtggcacaatctaggctcactgcaacctctgcctcctgggttcaagtgattctcctgccttagcctcccaggtagctgggattaaagtcgcctgccaccatgcctggctaatttttataattttagtagagatatggtttcaccatattgggcaggctggtctcgaactcctaacctcaggtgatctgcatgcctcagcctcccaaagtgctgggattacaggcatgagtgcctggctggaatcccagactttttttttttttttttaatttggagaaacctattttcaaaagatactgacctcaagcctctgactggatccaattcagttacttagatccaatcacatcccagatccactccactaaaattgctcaaataaagtcagagaggtcaaaacaaaaattcatagagcttgaatttaagagagaactctgccatgatcccagttgctgtgagagatgagtgggcacataatccctggtgggtaccttcacttggccataccgtgctcctgtgtgtccctggaggtctgctttgggtcccacttctgacaccaatctattaaaataaatactttagacaaataaaatttagtagagtttatttgagcaaagaagtggttcatgaattgggtagcaccttgaaccagtaacaattcagagagcttcacccagcaatagtggataggtagtatttatagacagaaaaagaaagtgacatatagaaataacctgatttgttacaactgtttatctgccttattttatcatggtctgagcagttgcagcctgtaattggctgaaatctcagttgctatgattggctgagacctagctacttgttacaagaatatactctcaagttagattgtagtttgtttacatattaagataggttacagtttactatgtatggagagaactttaggccatatttaatttaatagtatataataaagacccacgaacatcaaagaagtcctccgcctactagaagtaactgtcatagtggatcctgtgttcactttttttttgctttgatctcatctttatatataattctgtaaaaaaaatttcagaaagcactgttgttgagccccatgttaggcttgggagatcactactggctgaagagaaagtgttaggttcaaataataaaatctgttaagtgctagactgtagcaaatgcaaaaacctatagggtataaatgaaaaatgaagattgcatagacaaacttagtaacttgaagcatgagtcaatttatgaagaacagagaatttgcttcagaggaagcaacaaattcaaaggcagaagcatgagaaattaaagcaacaggaaaagggggtgaggaattaggtgtggttagagatgaagctaggatgatagattgggaggatgattaggtgggtttttagtcctcatttacaacttgtgtcaatccaaggccgaacatagtctaattacagtgttgtcatatattttttaatgaaatttgtgtgtaatggacagcagtgatgcttcagaatggtacattttcagatctgtaattattttccttgggaaagaaaaaatagccacttgtcttagtctacttgggatgctataacaaaataccactagtttggtgggttataaacaatagaaatttatttctcacaattctggaggcttggaagtccagatcaaggcacaagcagattcatcctccttcctggttcacagatgactccttctcattgtaacctcacgtgatagaagggacaagggagctagctctctggggtctcttttataaggtctaatcccattcatgagagcctgccctcatgacctaatcacctccctaaggctcacctccagatatcttcacattaggattcaacataagaattttgagaggacacaaacattcagtccacaatactgctgcagtattttttttttaacaattctcaaatctgaggtatctatagtatgtaaagatgtgatgtggccaggtgcagtggctcatgcctataatcccagcactttgagaggccaaggtggacagatcacttgggcccaggagtttgagaccagcctgggcaacatggtaaaaccctgtttctacaaaaaaaaaaaaaaaattagctggacatggtggtgcttgcctgtagtcccagctactagggaggctgcaatgggaggatcacctgagtccaggaaggtcaaggcaacagtcagctgtgatcatgccactgcactccagcctgggtgatagcgtgagaccctgtctcaaacaaaacaaaacaaaaagtgatgtaaagaaaggagcactgtagccagaattcagtgtcctggcatagtcactagtgtgccgtatgaccttctacaaacaccctcctttggctccaatgtcatctctcaaaagaaatagttggacaaacaacattccagctgttttctactaatagtctaacaccacttaccatcttctaaagtgggagggccaccactgcccctcctcctgcctagtgctgatcagatgacagttacaatagaatcattcttcttttgccagaagtgcaaatcatttcccaaatgtggccttgggttttcaggagcccagtagtgacttctgtggtccctgacttgcaccctcaccctattattggagttgtgcttcatttctctgtgtggaaagagtgaagtattgggaaatatgaccctggtcaccaaaccaactcagagacagcaagtgctttggcaaaggaaagggctgcacangagaacagagtaagtaatgactacttttatttttgtactataaatggataattatagtacttaaaaaatagacatcaaagaagcattatctaacaacatacatgatggtgggctctgagtgcagcagaagcagtgcgtaaagtaggtcctttgcctccaacagagcagtggtgggcaaaaaatatcaatgcttttctggtcttcaagctttatgaagtaggcaggcagagagagtcgagctgacacagtactgacactgtcaggtgatagatgcatcgacccaagcacaggattttttaacagccaggagcatttggggttgggattcattgagtaagttgggattttccttctatttgtgagaaattaataatttctggcttatggataaaaagtgaaaaattaatgttgcctctcctgttttgttgagcaatttaaaaatttcatatggcagaaaaccagaggctgtggctgctcagggtggcatgacagcaaagtccagaaagttcaatcaaaagcttacctactgcaaaattaaagatgggtgacattgccactcttgtcagcagagttttcaaggaaataatcatatgatttgcataattaagacatcattagttcagaagggaatgatcatgtatttgtatggcaaactgaaacatgctctcaggatgacttataagacatataaagtaaaatctctaataatactgaataacttatcaagtaattctgtgctcttgagcttttagaaaaaaaatttatcctggcagaccttaaatatcttggtttttctcatcatgctagcagttgttgtaataccaggtgctttctacctccattttctcctctacttagaaaacctttattccacttacagagcattatggccttccaaaataacgagttgaaattaaagtctagaaactgcgaagcataggattcctgggatcgaagtagtcaaaacacacgcaaaacaaacatttgtataaaatgctgcctttggaaaaaagaaggtgatgtgtagtacagcttatgcaatcttgattcagggttttaggcatattctgcaatctgggtgagaatttatttggactcttgctaaggagatgaaatgttcatttcaccccaaacacagattttgcacacagagggtgacaatagcctattttaccataagtattagtggctctggggtttcattgagtataggtcaggattctttttctattttatgggaagggaaggatttctgaattattgagaatgaactatttttctaacctcagccagcagtctccaaaatgcttgcatgagatcataagactcactaagttggacaatgagaaccatgcagtacacattcattactacccaaggggggaaccacctacagaatgtggcctttcacataaattataccttctgatttgttttcttaggatttccaggtatgaagttttatgcaatgaaaactaaccattcactagccttaaaaacaatgataataatgatggccatccctttgagcaatttttatataccactcagttaggttaaatggttttgcatgtaatatcagcatttattgagcccattgctatttttacaccattctgggtactacatgtgaattcattcattaaattctcacaataaccctatgtggtaggtgctaagggtacccatcttttacgggtaagaacgttgaggcccaaaaaggttcagaaacttagccaaggccacacagctaatgaatggcactggcagacttcaaattcaggacagtttgttttcaggggtcgtgttcttgactacatgctgttttgccttttcataacaaccctcaggctttcgtgctatcctcatctaaaatgaagaaacaggtttggagcatttcaaccatttattaaatgtagctcacgtaataactgaccaggctcagattttatcccactctgacctcaaggctctcatgcttttcacaattccatacttgttagacagtacccctgcttctagaggggaaaaaaggagggagatgttctccagtaaacattatctacgtttagatttggagcttttgctgttatagtaatattatcataaaaattaggccaattaatatatttggacattcttactaataaaccccagagctgcaagtttcccacagtgattcccaatggttctacctgaagactgtaagagtcatatgtttataccatctgatttccggcatgcagaatcttgtttctcaaacacaaacatccatttttaattgtttattgctcacctctccttgatacttcaaatacttatcagatacttcaaacgtaacttatcaaaatttgagcctattatttcctgcccccccacctcactcaaatagcttccacctcttcttgcatttgatgcctcagttcaaggggccttatgatgtaccgtgtatcagctgttagttactttttattgctgaatagtattccattgtatggctttaccagttttgtctatacatttagcagttgatagacattgaggttggttccagtttggaattatgaataatgctgctatgaacattcacaagcaaggagtcttgctatgttggccaggctggtctccaactcctctcaagcagtcctctcacctcagctttctgagtagctggaattacagacatgtaccactgcacccagcttggacgtacattttcatttctcttgggtagttttgtgtttaactttttgagaaactacaaaactgtttccaaaatgactacacaaattttataaccaccagcaatatatgagttcctttttctccacatcttcaccaacacttgttattttctgtttatctgttatagccatcctagtgtgtgtgaaaaggtacacttaattctcttccctaattactaatgatgttgagcatttttatgtgcttattagccattcgtgtatcttctttattaaaatatttcttctggggccaccactgcccctcttgcacttttgccagaagtgcagatcgtttcccaaatgtggccttgggctttcaggagcccagtagtgacttctgtggtccctgacttgcaccctcaccctgttattggagttgtgcttcatttctctgtgtggaaagagtgaagtattgggaagtatgaccctggtcaccgaaccaactcagagacttattttcaatatgtactcatgaattcttagtctttccaaaggtatataattcattactgtatttatttaattattttgattttccaattgaaccagatttggccagtgggaatctctccaagatggttcctttgtgacatttccccactatcttctcacttcttgattcctcaccatcttctcacttcctgatttttggacataacaaggctcatcttgtacctcttctgacctagctccagaaacagccattttcatttagaagctctggttcctctgagtggaaatggtattagaaaccaaggtctgggcacaaccgtaccttaaagagagtaatctggcagctgagtgaagtatgcattagttctctattaccctgtgtattagtctgttctcccgctgctatgaagaaataattgagactgggtaatttataaagaaaagaggtttaattgattcacagttccagatggctggggaggcctcaggaaacttacaatcatggcagaaggtgaaggggaagcaaggcgctttcctcacaaggcagcaggaaagagaagtgccaagaaaaggggggaaagccccttataaaaccatgagatcttgtgagaactcactatcatgagaacagcatggggtaactgcccccatgattcagttacctcccaccaggtccatcccatgacacgtggggattatgggaactacagttcaagatgagatttgggtggggacacagccaaaccatattactgtgtgacaaattaccacagctttgtggtcaaaaaaagaaaaaaaaaatctcacactttgttttgatcaaggaatcaggggtgacttagctgggtggctgtagctaatggttcttcatgaaactatagtcaaaatgttgtctaagtctgcaatcatctaaaggctcgacaggaacaggaatgtctgttttcgagatggcttgctcacatatggtacatggctgttgacaggatgtaccatattcttactggctgttagcaggaggccttggtgccttgtcacgtaagcatctccacagggctgcttgtgtgtcctcatgtgacagcagctggcttcccttggagcaagtgacccaaaagagcaaggcaggaaccacaatgtttttttatgacctagcttcaaaagctgcacatcatttctgcaatagcctagtggttatacaagtcagtgttattcagtgtgggagggaaccatataaagaatcatcaggggccatcttggactctggctaccacacaagcccaagatggtaaagagacttttctgtgttatttttatgttattttctatgctgtgttttacttttcacttgtaggtctatcacccgtcaggaattgtttttaagacatggtgtgagatagggagtcagggttcatttattcctttactgataggcagttaatatgcaccatttattgaaatttccactctttcccccagcatactgcaggggcatctttgtgacaaatcaggtgctcttatgtgtgtaggtctattggtctatttgtcttcgtgccagtgctaccctgccttgcttattgtagttttataaggagtcttgacatctgctaccatgattgttttgactattcttggccctttgcttttacaaataaattttatttagcgttggccactttttgcaattatttctgctcaggcctaaccctggtaaggttctgatttaattggcctggggtgaaatctaggcatcagtatttttatcaaacaccccaagtggttctaaaatactgccatagatgagaaccatggatctagaagaactctagtctgtggcagcagggaaaattaaggcccaagaaagttgagtgcttaccaaaagttacacagagaggcagttgcagaaccagggctccagcccaggtgccttgttttccagtgcagtcctctttccaccatctatatttactcctggacatgccactgtaggtaagtagcagaaaggcagagaggagagagttacccagagactttttaactccttacatctgagagcttcagaggaaatggcagttgggggacaacggagacattagattattagtttacagacacagccaagatgctcagcagggagggtgtaacaatggaataataaagctacacagagatctgagagatcatctttctaatctttgatagatgaagaaacagaccaaagagatcacatagctagttataagcaggggcaggacttgggtctgaagtgtgccacactcctggttcagtcctcattgtcccatcgttgtcatagactgacaggttcttcttgcctgctgctcagaaaaaccaatgcactgagaacagcaagtattgcagcaaagaaagagtttaattattgtggggccagacaaatgaggggaatgggagaaatttctcaaatctgtctctccaataattcagagggtaaagtttttaagggtaaattggtaggcaggtagtggggaaactgaaacaattgattggctgaagatgaaatcacagaggtgtctaaattgtcttcacacagctaagtcttcccagggaagggggaaagggtgttctcagaaccaagtggtatctctttgttgaaatgctaaatctgaaaaatatcttaaagaccagttctttaggtttcacaatagttatgttatctataggagtagctggggagttacaaatcttatgacctctcatgtgactttggagcagaaagcaatgtatagaaaagcaagctaagcagcagtaggttattgtttaaccatgcttattctttagcaaggttcaagccccaaccataattctaatcttgtgttatgaatgcagcattagtctccagcaagaggggattcagttttccttgcttcaaagtttaactataaactaaattcccttcatattatcttggcctccatgctagaataaacaacaacaaaaagaaagaaaaaagagaaaagaaaaagaggagaggaaaagagaagagcctgtgaggttgaggttaaaagcaagacagtcagtcatgttagatttctctcattacttaacaattctgcaaaggtagtttcactatgttcagagttatactgaggtgcatttagggaaaattcttcatcttaaagattttaacagtgttggcatatactaaccattcaacatattttttgaatgaataagtgaatagcttgaaatttatggctatttaaaaacaatgccatttcatgcagtaactcccaccacaggcttgagttgtggtcccttaggagtttctgtctccaacttatctttgtgtacctggtcagatgtttcctgagaggagcaagcctttattgtttttcctacaattacgtttcttattaatagggcagaaagaatatacagcatatacagatatacagctaaatggtattacattaatcccaggcccccagggaaaattatttaacaaccaatgtaactttttttttttttgagatagagtcttgctctgttgcccaggctggagtgcagtgagctatcttggctcactgcaacctctgcctcctgggttcaagcgattctcctgcctcagcctcccaagtagctgggactacaagcgtgtagcaccatgcccggctaatttttttttttgtatttttagtagagacagagtttcaccattttggccaggctggtctcgaactcctgacctcaggtaatctgctcaccttagcctcccaaagtgttggtattacaggcgtgagccactgcacccagcctccaatgtaacttttgcctttaagatttttttctttgagacagaaaaaaaaatcttcaaaattcttcatcttaaatattttaacagtgtttggcatatactaaccattcaacatatttcttgaatgaataagtgaatagctttacatttatgtatatttaaaaataataccatttaacgtaggtgcttgaaacaatatgaagtaagaatcctacctgcgattgtttttggtaggtccttaagatttggcagcttactctcaatatatctctcttactctctctctctctctctcttttttttttttaatatggtgaaattagaccaggggtcagaacatagattttagtctcctttagttcatctactaggagactaaattagataatctctaaactcccttttagttctaaaattctgtaattaaactctagcatatcatcattttagactaaaagttttcttcttcttcttctttttttttttgtttttttgagatggagtctcgctctgtcacccaggctggagtgccatggtgcaatctcggctcactgccacctctgactcccgagttcaagcgattctcctgcctcagcctcctgagtagctgggactacaggtgtgtaccaccatgcctggctaatttttgtatttttagtagagatggggtttcactgtgctggccaggctggtctggaactcctgacctcaagtgatccacccgtctcagcctcccaaagtgctgggattacaggcatgagccaccgcacccggcctagactaaagatgttttcaattaaacattcttagtatgctttagtaagagctattgaggctttcattttacagatgagtattaaagtatcatgaaccaaataggctctgtgctataagaaataactcaataagaaataactcaatggtggttcatgcctgtaatcctagcacattgggaggccaaggtgggaggatcgtttgagcccatggattcaagaccagcctgggcaacatggtgagaccctccccccccccgccccatctaaaaaaaagtacaaaaattagctgggcacattggtgcatgcctatggtcctaggtcttcaagagacagaggtgggaggctcgctccagcccaggatgtccaggctgctgtgagccatgatcgcgccacgacactccagcctggatgacagagtgagaccctgtctcagaaaaaaaaaaagaaagaaataactcaattgtttccaggcaagctatcgcaatctggtaggacattgttcctttattttttcaaggaatagatgttcgttatagaaaatttgagaaatgtagaaaagaattaagagtaaaacaaaaatcaaacatatacattgtaagtcagatcatgtcccttctctgctaaaaactctcccatagctcccattttattcagataagagcaagtctttaccccacgcactcccccgcaccactatccgctactctcttttgtccattcactctgcttcagctgtgctgtttccctgctgctgttcctcagaccttgcaggcacacgctcactatgagggctttggacttgctgttccctctgcctggggtgttcctttcctagatatctacagggctcacacctcaattccttggctgttcacttaaaaatcaccctcccagtgaggccaccttccctggtctcgccacctaaaattgtaacctctccctgacattttgtatccccttgctagttttatttcttaacacttatcactaacatgctacacattttgtttctcttatccattcaactttttttcccctttcaggaattaagcctcttcttttttttttcttttgagatggagtctcactctgtcacccaggctggagagtggtggtgcaatctctgctcactgtcacctccacctcctggggttcaagcaattctcctgcctctgcctctcgagtagctgggattgcaggtgcctgacaccacgcccagctaattttttgtatttttagtaaagacagggtttcaccatgttagccaagctggtctcgaactcctgacatcaagcgatccacctacctctgcctcccaaagtgctgggattacaggcatgagccacaatgcctggccaggaattaagcttcttacatcacagatttttcttttgttcactgctgtgccccaacatctagaacaggcctcataattgttttgtgcatggatgagggtgggtattccattgaccatagtgttgagcatgtggattttttatccctttttttcttttctttccatttctcttctccattgctctcaaatatatcactgcaatgaataacgtatcccaaactatttttcatctgattatctcccatagttagtatcttagagtagaatttattgggctaaatgacataaatgcctaatttttaaaatttggggatgattttaagttacagaaaaattgcaaaaatggtccaaagttcttatatttccttcactcaacctctactaattttgacatcttacataaccatggttcatttgtcaaaactaagaaattaacattggtatgatactattaactaaactacagatgttatttgagttcaccagtttttccactaaagtcagtattctgttccaggattcaatctaggatgccactgtgcatttaggaataattagaatcttggtacatattcttaaattttctccagatatgtggttccagtctgtattgccaacagcaatgtatgactgcttctctcttatcaatacttagtattttcctttttcaaaaatatctttaaaattcaatggttaaaaatggtgtctcactgtaatagttccatttcaatgaatttgaaaaatgttttatattattggccatttgtattcctaaaaatttttggccaggcatggtggctgatgcctgtaatcccagcactttggaggctgaggcaggtggatcacttgaggccaggagttcgagaccagtctggccaacagggtgaaaccttgtctctactaaaaatataaaaattagctgggggtggtggcacgtgcctgtaatcccagttactcgggaggctgaggcacaagaattgcttgaacctaggaggtggaggttgcagtgaatcgagtttgagccactgcactccagcctgggtgacagagagagactgtctcaaaaaaaaaattatatctcaaaaattaactcaagatggattaaagacttaaatgtaaaacccaaaactataaaaactctagaagataacctaggcaataccattcaggacataggcactggcaaagatttcatgataaacataccaaaagcaattgcaacaaaagcaaaaattgacaaatgggatctaattaaactaaagaggttctatacagcaaaagagactatcaacagagtaaacagacaacccacagaatgggagaaaaattttgcaaactacattcaacaaaggtctgatatcaaaagtctataaggaacttaaacaaatttacaagaaaaaacaaacaatctcattaaaaagtgggcaaaggatatgaacagacacttctcaaaagaagacatacatgtggccaacaattatataaaaaagctcagtatcgctaatcattagagaaatgcaaatcaaaaccacagtgagaaaccatttcacaccagtaagattggctgttaagaaaataaaaaaataacagatgctagtgaggttgtggagaaaaaggaatgcttatacattgttgatgggagtgtaaattagttcaaccattgtggaagacagtgtggtgattcctcagagacctaaagacagaactactttttgagccagcaatctattactggatatatacccaaaggaatataaatcattatattataaagacacatgcacacatgttcattgcagcactattcacaatagcaaaaacatggagtcaatctaaatgcccatcaatgatagactgggtaaagaaaatgtggtacatatacaacatggaatactatgcagccataaaaaagaatgagatcatatccttcacagggatgtggatggagctggaggccattatccttagcaaactaatgcaggaacagaaaaccaaatacctcatgctctcacttataagtgggagctaaatgataagaacacatggacacatagaggggaacaacacacactggggcctatctcaaggtggagggtgggaggagggagaggatcaggaaaaatcactaatgggtactaggtttaatacctgggtgatgaaataatctgtacaataaacccccatagaagtgactatgttgtctggggtatataccctggggttcatggtcgtgcaccaggaaaatgttaagacatggacaaacaagaggagtttaggagtggaggtttaataggcagaagagaagagaaagagaaacagctttctctatagagagagtggggtccctgaatggaaaagacctgcaggtggcattgaattttatagtcaggtttgaggaggcagtgtctgatttacaaagggttcacagataggttcgatcaggtatgacatttacatagcagatggggaaaggccggttgtctcaccctaatcttattatgcaaatgggctttccagttgatctgagccatcttgtctgcttcttactgtacacatggctggcagagaagggaagatgaagctgccatcttaaacatgtctagcccctagttcctgccagcattcacccatgcaagctcccagcttcctcgtctatgtctgccgcttgactttacaggctgctctttgttagacaatgatttggggctgcttttcattaaagagaaaagccttaccgaggacttctgtaccctcactatctgcctaagtgatttcttcttaactcctgtatcaccatgacacagttatcctatgtaacaaacctgcacatgtacccctgaacttaaaagtttaacacaataatttgtatcttttgcctgtttttaaaattagggtattctactactgaattatagcacttacagttcactagaagaaatactatgctatagtatatgaaaatattaaagatactatatattatagtatatatggatttataagcactgacttataagcactttatatattaaggatattgaatttttggcaaggctgatacaaacattttttcatgatttgccagccgttttttggaatacaagagttttctatttttatgtcattaagtgtatgctgttttagttttctgattgattctattatttttttaagagttggattttttctttttttctttaaacagccttattgaagaacattgatggataaaaattgtacatatttaaggtatacaacttgatgttttgatgtatgtatacattgtgaaatggtcttcacaatcaaaccaattaacatatctatcacctcaccaagttaccattttctttttcttcttttattattatctttttttgtggcaaaaacatttaagatctacttcttagaaaatttcaagtaaggaatattgttaatcatagccaccaggctgtacattagatctgtagaacgtattcatcctgcatattgaaactttttatcctttgaccagcattaccgtttctccctccctactgcccctggcaaccaccattcgctttataattctatgagtgcgactattttaggtttcacacatcaatgggatcaagcagtatttgtttttctgtgtctgccttatttcatttagcataatatcctccaggttcatccatgttgttgcaaatggcaaaatttcattctttcctgaggctgaataatattccattgtgtgtgtgtgtgtgtgtgtgtgtgtgtgtgtgtgtacaccacattttcttttatccattcatccatcagtggatacttacactcttttcatatcttagctgttgtgaataatgctgcagtgaacatgggagtgcagataaatcttcaagatcttaatcttgatttccatttctgtgggtatatcccaaaagagggattgctagataatatggtcgttctattttttattttttgaggaccctgtatactgtttttgataaaggctgtcccaatataaattctcatgaacactgtacaagggttccctttttcctgcatctttgtcaacacttttaatctcttgtctttttgattaatagccatcctgatagttatgaggtgatgtttcattgggttttcatctgcatttccctgatgattagtgatgttgagcaccattacatatacctgttggccatttgtatatcttctttctagaaatgcctatttaggtcctttgcccattttataattggattatttgcttttgctattgactcgtgtgagttccttatatattttggatattaactccttgtaagatatatggtttacaaatattttctcccattccataggttgttatttaactctgtgatttttttttctttgctgggcagcagttttttaatttgatgtgattctacttgtctatttttgcttttgttgcctgtactcttgctatcttattcaaacatattattgccaagacaaatgtcagaaagctttttccctatgctttcttctagtagttttacggttctgagtcttatgtttaagtttttaatccattttgagtttatttttgtttatggtgtcagatgagggtctaatttcattcttttgcctgtgaatatccagttttcccaacattagttattgaagagactatcctttcaccattgtgtgctcttggcactctagtgaagattgactgtgaatgcatggatttatttatgggctctctattctgttccgttaatctatatgtctgtttttatgtcagtaccatcgcacttaggttactgtagctttgaaatatcttttgaaatcaggaagtgtgatgtcgccagctttattattctttctcaagattgctttcaacctgggcaacatgtcaaatctccatctctacacacacacacaaaatacaaaaattagctgggtgtggtaatgtgcatctgtagttccagctattcaggggggtgggggggtgggggggaaggatcacttgagccccaaggctggagtgagccatgtttgcgccaccacactccaacctgggtgacagagcaagaccctgtcttaaaaaaaaaaaaaaaaaaaaaaaaaaaaaccaagcaagattgctttggctgtttggggtcttttgaagttccacatgaattttaggattgtttttctctatgcgtgtgtagggagtgctgtttgaaatttgttagggattgcattgaatctgtagatcattttggatagtataaacactaattcacaaacatgggatgtccttccatttatctaatcttctttaatttccttcatcattgttttgtacttttgagtgtacaagtctttcccctctttaagtttatttctatgtattttattctttttgctgctattatatatgggaatgtttgcctatttcctttttggataatttattgttagtgtatagaaacactgctgaattttttatgttgattttatatcttgcaactttattgaatttgctaattagttctaacagatttttttgttgagcctttagggttttttacatgtgtaaataatattttctttttttttttttttgagacagggtctcactctgtcgcccaggctggagtgcagtggcgcgatctcggctccctgcaacctttgcctcccgggttcaagcatttctcctgcttcaacctccctagtacctgggattacaggcacctgccaccacgcctggttaatttttgtatttttagtagaggcagggtttcaccatgttggccacactggtcttgaactcctgacctcaagtgatctgccttccttgtcctcccaaagtgctggaattacaggcatgagccaccatgcctagccatgtgtaaataatcttgtcatctgcagagataattttatttcttcctcttcaacttggatgcctttttatttcttttttctgtctagttgttttggttaggttttccaacattatattgataatagtggtaaaagtgggcatcccttccttgtatcagatcttagaggaaaagctttcagtttttctccattgattatgatgttagctgtgacctttacatatatggcttttactgtgttgaggtatgttccttctatacctatattgttgaaagttttttatcatgaaaggatgtcaaattttgtcaaatgctttttctgttatctattaagatgatcatgtggttttttacatgtcattctgttaatgtagtatatggcattgattgatttgcatatactgaaccattcttgcattccagagataaatcccacttgttcgtggtgtatgatcttttaaatatgctgttgaatttggtttgctggtattacattgaggatttttatggctatgttcatcagtgatgttggcctatggttttcttttcttttgatatctttggctttcgtattggggtgatgctggcctttaaagtgagtatgaacgtgttccctattctattttttaaggagtttaagagggattggtattagttcttctctgaatgttagaaaaagccatggtcctgggcttttctttgttaggaggtatttgattactgattcaatgttcttatttattttcagaatttaatttcttcttaatttagttttggcaggttgtatgttactagggattttctgtttcttctaggttatccaatttgttgctgtatagttgttcataagttgtctcttataaccttttaatttctgagtcatccattgtaatgtcttctttcttatttctgatttgtcttttttttatagttcaattttttttttattatactttaagttctggggtacatgtgcacaacatgcaggtttgttacatatgtatacatgtcccatgttggtgtgctgcacctgttaactcatcatttacattaggtgtatctcctaatgctatccctcccccctccccctaccccatgacaagccccagtgtgtgatgttccccaccgtgtccaagtgttctcattgttcaattcccacctatgagtgagaatatgcggtgtttggttttctgtccttgtgatagtttgttcagaatgatagtttccagcttcatccatgtccctgcaaaggacatgaactcatccttttttatggctgcatagtattccatgttgtatatgtgccacattttcttaatccagtctatcattgatggacatttgggttggttccaagtctttgctattgtgaatagtgctgcaataaacatatgtgttgatgtgtctttatagcagcatgatttataatcctttgggtatatacccagtaatgggatggctgggtcaaatgatatttctagttctagatccttgaggaatcgccacactgtcttccacaatggttgaactagtttacagtcccaccaacagtgtaaaagtgttcctatttctccacatcctttccagcacctgttgtttcctgactttttagtgatcgccattctaactggtgtgagatggtatctcactgatttgcatttggttttgatttgcatttgtctgatggccagtgatgatgagcattttttcatgtgtctcatggctgcataaatgtcttcttttgagaagtgtctgttcatatccttcacccactttttgatggggttgtttgatttttttcttgtaaatttgtttaagttctttgtagattctggatattagccctttgtcagatgggtagattgtaaaaattttctcccattctctaggttgcctgttcactctgatggtagtttcttttgctgtgcagaagctctttagtttaattagatctcatttgtcaattttggcatttgttgccattgcttttggtgtttgagtcatgaagtcattgcccatgtctatgtcctgaatggtattgcctaggttttcttctagggtttttatggttttaggtctaacatttaagtctctaatccatcttgcattaatttttgtataaggtgtaaggaagggatacagtttcagctttctacatatggctagccagttttcccagcaccatttattaaataaggaatccttccccacttcttgtttttgtcaggtttgtcaaagatcagatggttggagatgtgtggtattatttctgagggctctgttctgttccattggtctatatctctgttttggtaccaataccatgctgttttggttactgtagccttgtagtatagtttgaagtcaggtagcatgatgcttccagctttgttcttttggcttaggattgtcttgccattgtgggctgttttttggttccatatgaactttaaagtagttttttccaattctgtgaagaaagtcattggtagctggatggggatgtcattgaatctataaattaccttgggcagtatggccattttcactatattgattcttcctatccatgagcatggaacattcttccatttgtttgtgtcgtcttttatttcgttgagcagtggtttgtaattctccttgaagaggtccttcacatcccttgtaagttggattcctaggtattttattctctttgaggcaattgtgaatgggagttcactcatgatttggctctctgtttgtctgttattggtgtataagaatgcttgtgatttttgcacattgattttgtatccagagactttgctaaaggtgcttatcagcctaaggagattttgggctgagactatggggttttctaaatatacaatcatataatctgcaaacagggatgatttgagttcctcttttcctaattgagtaccgtttatttctttctcctgcctgattgccctggccagaacttccaacactatgttgaatagaagtggtgagagagggcatccctgtcttgtgccaagagaatgcttccagtttttgcccattcagtatgatattggctgtgggtttgtcagaaataactcttattattttgagatgtgtcccatcaatatctagtttattgagagtttttagcatgaagggctgttgaattttgttgaaggccttttctgcatctattgagacaatcatctgattttggtctttggttctgtttatatgatggattacatttcttgatttgtgtatattgaagcagccttgcatcccagggatgaagccaacttgatcatggtggataagctttttgatgtgctgctagattcggtttgccagtattttattgaggatttttgcctcgatgttcatcagggatattggtctaaaattctctttttttgttgtctctctgccaggctttggtatcagtatgatgctggcctcataaaacaagttagggaggattccctctttttctattgattggaataggttcagcaggaatggtaacagctcctctttgtacctctggtagaatttggctatgaatccgcctggtcctggccttttttgattggtaggctattaattattgcctcaatttcagaacctgttattggtctattcagggattcaacttcttcctggtttagtcttgggagggtgtatgtgtccaggaatttatccatttcttctagattttctagtttatttgtgtagaggtgtttatagtattctctgatggtagtttgtatttctgtggaatcggtggtgatatcccctttatcattttttattgcatctatttgattcttctctcttttcttctttattagtcttgctagcggtctatctattttgttgatcttttcaaaaaatcagctcctggattcatcaattttttgaagggatttttgtgcctgtatctccttcagttctggtctgatctgttatttcttgccttctgctagcttttgaatgtgtttgctcttgcttcgctagttcttttaattgtgatgttagggtgtcaagtttagatctttcctgctttctcttgtgggcatttagtgctataaatttccctttacacactgctttaaatgtgtcccaaagattctggtatgttgtgtctttgttctcattggtttcaaggaacatctttatttctgccttcattttgttatgtacccagtcgtcattcaggagcaggttgttcagtttccatgtagttgagcggttttgagttagtttcttaatcctgagttctagtttgattgcactgtggtctgagagacagtttgttataatttctgttcttttacatttgctgaggagtgctttacttccaactacgtggtcaattttggaataagtgcaatgtggtactgagaagaatgtatactctgttgacttgggttggagagttctatagatgtctattaggtccacttggtgcagagctgagttcaattcctggatatccttgttaacttcctgtctcgttgatctgtctaatgttgacagtggggtgttaaagtctcccattattattgtgtgggagtctaagtctctttgtaggtctctaaggacttgctttatgaaaatgggtgctcctgcattgggtgcatatatatttaggatagttagctcttcttgttgaattgatccctttaccattacgtaatggccttctttgtctcttttgatctttgttggtttaaagtctgttttatcagagactaggattggaattgctgctttttttttttttccatttgcttggtagatcttcctccatccctttattttgagcctatgtgtgtctccgcacgtgagatgggtctcctgaatacagcacactgatgggtcttgactctttatccaatttgccagtctgtgtcttttaattggggcatttagcccatttacgttaaaggttaatattgttatgtgtgaatttgatcttatcattatgatgttagctagttattttgctcgttagttgatgcagtttcttcctaccagcgatggtctttacaatttggcgtatttttgcagtggctggtacctgttattcctttccatgtttagtgcttcctttaggagcacttgtaaggcaggcctggtggtgacaaaatctcttagcattagcttgtctggtaaaggattttatttctccttcacttatgaaacttagtttggctggatatgaaattctgcattgaaaatttttttctttaagaatgttgaatattggcccccactctcttctggcttgtagagtttctgccgagagagtcgttgttattctgatgggcttccctttgaggataacccgacctttctctctggctgcccttaacattttttccttcatttcaactttggtgaacctgacaattgtgtgtcttggagttgctcttctcgaggagtatctttgtagcattgtctgtatttcctgaatttgaatgttggcctgccttgctaggttggggaagttctcctagataatatcctgcagagtgttttccaacttggttccattctccccgtcactttcaggtacaccagtcagacgtagatttggtctttttacctcgtcccatatttcttggaggctttgtttgtttctttttacacttttttctctaaacttctcttctcgcttcatttcattcatttgctcttcaatcactgataccctttcttccagttgatcgaatcggctactgaagcttgtacatgcgtcacgtagttcttgtgccatggtttttagctccatcaggtcatttaaggtcttctctacactgtttattctagttagccattcatgtaatattttttcaaggtttttagcttctttgcaatgggttcgaacattccctttagcttggagaagtttgttattaccaatcatctgaagccttcttctctcaattcatcaaagtcattctctgtccagctttgttccgttgctgacgaggaactgtgttcctttggaggagaagaggtgctctgatttttagaattttcagcttttcttctctggtttctccccatctttgtggttttatctacctttggtccttgatgatggtgaatacagatgggattttggtgtggatgtcctttctgtttgttagttttctttctaacagtcagaaccctcagctgcatgtctgttggagtttgctgagggtccactccagatcctgtttgcctgggtggcaccaccagaggctgcagaacagcaaatgttgctgcttgaaccttcctgtggaagctttgtctcagaggggcacccggctgtatgaggtgtcagtaagcccttactggtaggtatctcccagttaggctactcgagggtcagggacccacttgaggaggcagtctgtccattctcagatctcaaactctgtgctgggagaaccactactcttttcaaagctgtcagacagggacgttaaagtctgtagaagtttctgctgccttttgttcagctattccctgtccccagaggtggagtctacagaggcaggcaggcctccttgagctgcggtgggctccacccagttcgagcttccaggccactttgtttacctactcaagcctcagcaatggcggacgcccttcccccagccttgcagttcaatctcagactgctgtgctagcagtgagcgaggctctgtgggcgtgggaccctctgaaccaggtgcgggatataatctcctggtgtgccgtttgctaagacagttggaaaagcgcagtattggggtgggagtgtcccgattttccaggtaccatctgtcacggcttcccttgtctaggaaagggaattccccgaccccttgcgcttcccaggtgaggtgatgccccgccctgctccgtgggctgcacccactgtccgacaagccccagtgaaatgaacccggtacctcagttggaaatgcagaaatcacccgtcttctgtgttgctcacactgggagctgtagactggagcttttcctattcggccgtcttggaacctcttgtcttcttttttttcttagtttagttgagggtttgttgattttatctttctaaaaaatcaactcttggttttgttgatttttctattgcttttctattttctatttatttctgctgtaatctttattatttcctttcctccactaactttgagcttagtttggtttgttctgcctggtctcttgaggtataaagtcaggctgtttatttgagatctttctcctttaacgtatgcatttatcactataaactcctctcataacacagcttttactgcatcccctacattttagtatgttgtgtttaggtttttatttgtccaaagatatttcttgaatttccctttgatttcttctttgacccaatctttattagagtgtattgttttgttttcccatatttgtgaattttttcattttcttaccgtttttgatttctagtttcattccattgtggttagaaaagatgtttggtatgatttccattttttttacatttgttaagacttgttttgttactcaatgtatgatctatcctggagaatgttttgtgtacacatgagaagaatgtatattcttctgttggatggaaagttctatatttgtctgttaggtccatctggtccatagtgttgttcaagtcacctgtttttattttaattgattttctgtccagatgttgtatccattattgaaagcagggtgttgaagtctcctactgttattgtattactgtccatttctctctttagatagatcaatatttgcttttatatatctaggtacactgatgttggatgtgtatatatttataattgtatcttcttgttcagttggccctttcatcattatataatggtcttttttgtctctagttacagttttttacttaaagtctatttcatctggcatgagtatagccatccttgctctcttttaattaacatatgcatggaatatctttttccattgcttcactttcagcctatgtgtgtccttaaatctgaagtgagtctcttgtagacaacatacagttgaatcttctttttaaatccattcagttactctatgtcttttgactggggaggttaagtctatgtacagttaaagtaattatcaataggtaaaggcttactattgccagtctgataactgtttctggttattttgttgctctgtattcatttctccctctctttctgtcttcccttgtgatttgattatgttttgggcttgcataaagcatcttatagttacaactgtctatttcaagctgatactaacttcaatctcatacaaaaacgccattctccccaacctttgtgtccttgaattcagtgtttaattatctttatagtgtgtatccattaacaattgtgtgtgtgtgtgtgtgtgtgtgtgtgtgtgtgtgtgtgtgttttagacaaagtcttgctctgtcaccaggctggagtgcagtggcgtgatctcagctcactgcaacctccaactcccttggtttaagcgattctcctgcctcagcctcccaagtagctgggattacaggcatgtgccaccatgcccagctaattttggtatttttagtagagatggggtttcaccatgttggccaggatggtcttaatttcctgacctcatgatctgcctgcctcagcctcccaaagtgctggtattacaggtgtgagccactgggcccagccatcttttaacttttatattagggtaaaaagtaatttatgtactattatagtgttacattattctatatttccctgtatattttcttttaccagtgagatttatgttttcatgttgctatttaacatgttttcattttaatttgaagaatttccttaagtatttcttataagacaggtctagtggtaacaaacagattccctcagtttcatttgtcttgggatgactttatcctttatttttaaaggctatttttgcccaatgcaatattcttgtttggcagtttttttcttttagaattttgaatacatcatcctactctatccttggctgcagggattttgctgagcaatccactgagagtcttatggataattagccttttcccttaacaaaaactgattatttgaccctcatacaaagggaagaactgataagaatatgaaatatcttagccaggcatggtggcacacacctgtagtcctaggtacttaggaggctgaggtgggaggatcacttcagcctgcagtgagccgtgatcatgccactgcaccctagcctgggcaacagagtgagaccctgtctcaaaaaaaataaataaaataatataaaagtatatcatttcaatgatgataataccaaattatttactaatgaacaaataccctacttaaatgaatttggtttctaaaaacagtgcagcaaaaccgaagtagatttgagggtgccacattatttggtgttccccttgtttcactactgttatttcaccagaactcatactctggtggcacaagccacagtcactgaggcagcagacagaaaacaatgacaggtttgcctgtatcatcacctctgtagcctaaagggtcaaataatcaccttccatcaaactcacttaattcttcatttgcccaacatatttactgatgcctgctctgcataaggcatccactcttggtaagttatcagatatagtcagtccctgacatcctcatggagtggagatgagagagaggatagaaacagaaggtttaaataaacaaggtagttgcagttagtgatgagggctctggaggaaattgggtgatgagattagggagttctcaggaaaggccactttggatgtcatggtgggtgacctctctgaggagatgctgtctgagctgtgacttcaggaagaagagtaggctgtggaaaaagctggcagggggtccctccacaggcaaaatatggttcaagtatgaaaaagctctttttcctgagctgggaagtgtgagaccagcactcaatacttggccacagcaggctcatgtgggcccatctcctctcaactcaactcatgctgggcttctacagggaatataagacacctgggtggtgatgcagtcctctaaggaacttcactccatggggaaagttttaaagaattacccttgtttctaaggaccatgcttacattctattgtggacatctgtctttggaggcctattttcttttctttttacattaacttgacatctggtaaaacaaaattttgcgtagcaattaaatcaaaacaaaaaacagacatgacactttctcagttaaaatagtttaataaaagcaacaaaactgtgctaacgatgagaatcaaaaatgagatattaggtagacttataaaacaaagtatagttattttttgatttcaaataaaccatgtgcaaaattgtaaaatgccaatgtgtctgagaaaagcattaacagtccttttagcaatttatatataaagatgtttttaaagtgccacagcttaaggcattatattttaaagtttaataaacatctaatttcaacatctctccaagaacagacttcttctcaataagctataaactatttggttaggaatattgaaaatgcatgtataatttaaggagtaatatacttgttaatgctgagttattagtgcaattcaaaagcatatgaattccatatcaagaacaaactctcccgcccaaggtacagtgtaatccacactgtatcatctcatctaaaaatctatacagcagctaccccatccactcagttcctctgcagttaggctattagcttttctttttcaaaaagcaaaaattcctaagacacctaaagattagcctgtatttcatttatctatactgaaagtgcttagtaatatttctaaaaaggaaaagagcagtatggtagataaagaatgtagagtcaaaaatcaatcattttaaaatttttcttcttcctatgattatgttttggttaagcagatattattttcattttttgagcttgcaaaagtctgcctaggaatgtgctaaatcaaaggaaaaatctagcctcatgttcacaattgccctggaatgccattcccagactgagatctaactacacagagtatggctaacggcagaagtcagaggttagggagatctggtgtctccatttatctggaaaacagagcaaagaagggtgatcagttatagaaggccaaacagaagtgttttaagttcagaatttcatctttcgtttaattttcaaagtaacagccactctggatcttttcttgccctcttctctatcagtatgaacagcgtagctgcttctctcctttaggaaagtcagtgtgaggtccctggatatggcctagtctccaggtggtgggagtttacattctgttacctataaacagctaaggcatcgttctaagtttgtttaaaatggttgttttaaatggtaaacacaaaagtccagtgatttttttaaaaactggctttaatggacattaacaaataatatacactgatttatcacctttaagcaacaaaaacatgacttgtaattattcaaataaggtaggatttttctcttaagtacacttcttaaaagtcattcacaagacaactgggcatccactaagaccaaggcactgtgagggaggcaaacagcacaacatcctcacctcaaggagctcagcctgggatgaagacagacacacacaactccagcatgaggccaaggggtagcctgttatgggatcaagtggtggcagaatcaagaagtggttctgaaagtgttctttagtcacagagaccagtaggtttgaaacccagtgatgttactttttaactttgtgccttacctactataagcctcagcttcatccactacagtatgccaccctcttaatagaatttatgtaagaattacagaaacatacaagtaaaggattaagtgtagagccagcatgaggaaaggttcaggaatgtggttgctcatatgactgctatgcttacttctgtacatggagcactgtgggagaataaaagaaaggggtggtcattccttagagcatgttctcactgggggaaatcctggcatcttgagcaggacaattcttcactgtataggactaccttgacatttagcatcctggtcccatggcagcagaaggccagtagcacccccatcattatgataaccaagaatcatacccacacatttccaaactccctctgaaggggttaccacccctgctgaaaactgcttttccagagggaccatctacatttagaaacaatttagttctcttaataaaacgagagtgaattctttgagagtatattcaaggatcagatgcattgggcctggtggcagggacgctaggacaggaatgttcagtatagtttgaaagcagaatcctttcctgatgagtcccagtatcaaaatccccatgctcaccattcaggccttaagtaaaaaggtagagctgcatctttgcaaataactcagtagaaaggaattctgccaagaagctactaaagaagtagcaaggctcactgttcctgtagttgtagtagaagttagacatataaatttagataataatcctacaagggattttttaaaattataatttcttttttcctcaatatacacgacagagtccacttcatcccacatctacaaaaagtaaaaactaaaatcataaacaaaaagatagtgacagaacaagcccaaagaagctggcatagaatctgtgctgagaaaagttctcccccagtataacctaaatccacacccatctctgacaaagcattccaggctgaacaccactttcatgttgtcataggtctttaaaagccagctgatagatcttcttctcctgaagttcttctccagaacttaacagcattttagagatatcagaaaagatgtaaggcatttgaccatcattagtgctgagaggtagagagaagccaagttttattactctcattttacatatgggatgtctgaggcactaagaaatgagaagattggcccatcggtggaagccactgcaagtaaaatccagatatcccgtttcataggtactttgttttctatcccccaaatcacagacctgcattactaaaatggctgaagtatcctgctgaggaatcctccaagatgaaacctcccagtgtgctctaccctccttccgacctctgagcccagcttggatccactgagttgctggattcactgtcctttgcagtaggaagaatctgcttagaaggatatgtatttatatatctttttaccatctcttcttggagatgctcttcatctccttgaaatagttatattaaatggatgtggggctaaagttagaccaaatcttccagtcaggaggggcttcttagaatcctcaggtaaagcaaatgcgaaactctgcattaggctcacaaacattaggaataattccatctttgccagttgttctcccatacacacccgcttccctaaaaatgagatcagaaaaaaatgaaggattataataccatcccttctcctatttcaaaaagtacaaatttatatgaaatatctaaattgagtattctaaatgcagatggagtttttaagtcaaattgccatatataccacatagcctcccacagcatatgctgatcagaatttttacatatcaaatccacattttaactcttactcataaaaacctatccccattctagacaggaactagagacagatccagtgtggcagctatcctgtctcttctgtgcctacttacctctttcctccacttttgcctggaagattaaggcaaaaaaattcagataagaatccctagagcctccctttttccacagaaagaattctatgttcctaatctaggaacctaaaagaggaactgttgagattagagaacccatcctctcacccaccagaccagatcagaaattgctcttaagtagctccctgggaccccccaaaattacctgatcagctctgaaagtaaaatttctagtcacatttagagatttaaaaccaactgctttattaaaatctgaaatgaacataggggagatgccttgtctaaattattgatcccttctcttatattagcttgccaagaaagaaacatttttaaaacaacatgaaataataaaatattattttagttctacttcttttaaaaattatgcagtttaaaaaagtgtaactgacctatcccaaaaggaataaaggtttcttttttaattagttgtccttggtcatccagaaatcgattagggtagaaatcctccggtttctcccaaatggctgggtctctatgtactgaccacaggttgggtaagatcaatgtgcctttaggaatggtatacccttggagcactaaaacaaaaggaaacatatgggtgttattcagtatgcttttactagtagttctccctgaacatcattatctgaaaaaaaaatgtagttatatacctatcactttaattggctgagaccaaactgtgaggtacttgagggcagggataagatcttaccaactggcatccctctccccgcaccattccaggacccacctgagacagggagcaggcagtgctccaatgaatgctgaattaataaataaaaccataaaaacattacaatgaagagaaaacactaaaacagaaacaataggggatttcaagtaaacgttccctaaaaattaaaattcccaatatgtataaaacttagtttacagggtcatttatgtccatataacaatgtaatgaggaggtaaactaagataaagaaatatacagggtagccaaccctggctcttaccagctgacaagtcaatatgcgctttaaacaatgcccaaaagatactataaacagtctatctaacttgtaccaaaaaaaacccgttaagtgatccaatgtcatgtataagcaacggcatgctataactgtcttaaaactagatcaagcccattatactatgggctgactccagaggtaaattctttgttaatagggatgtttaaagagagtctggacatgaccaaatagacagagttcactgtagatgagaggctgaaacaggtgactttagaaaaagatgggctcttctaactatgatgatgatgatgatgatgattattattattattgagacaggctgtctcactctgtcacccaggctgaagtgcagaagtgcagtggtgcaatcttggctcaccgcaacctctgcctcccgggttcaagtgattcttctcctgcctcagcctcctgagtagctgggactacagaagtgtgccaccatgcctggctaatttttgtatttttagtagagatgggttttcgccatgttggctaggctggtctcgaactcctggcctcaagtgatccatcctcctgggcctcccaaagtgctgggattacaggtgtgagccaccgcacccagccctttttttttttttttttttgagacagaggcttgctccatcacccaggctagagtgcagtggtgcaatctcagctcactgcaacctcccaggtccaagcgattctcctgcctcagcctcctgagtagctgggactacagttgtgcaccaccatgcctggctaatttctgtatttttagtagggatggggtttcaccatcttgtccaggctggtctcgaactcatgacctcaagtgatccacctgcctcgtcctcccaaaattctgggattacaggtgtaggccaccacacccggccttctaattatttcatatttatgcttcagctgcagaattttaaaagttgaagtaagataattttcaggcaaaatccttaatccacatttagatgagctcttctgacctccatgaaaaggagggagaggtgcctttgtcaattctggagctttttctgccaacatagccaatttaaattttgacattatttgttttgccagtataattgtgtatgctctgaaggctacagaactactagtaaaactaagcagtagtagaattctagtacaaccagtgatgcacagcggagggctgaacattccctagacattttattacatatgacacaatgctaaatcttcagaaggatcaagctagacaacagtcaccacacccctcacctcaactaccctggattagagggccctgcttccgtcaagggcattcaaagaggaagaccctggacttgcctgtgttctctgaggtcatatgaggaatggcaagcggcaccaccacagttagcctctgcacttccatgatggtggcttctgtgtagggcatctgggccttgtctgtgagggaaggagctcggttggcgccaatgactctttcaatttcttcatgaaccttttctatgtaaaaagggaaaaataaaccagaagataagcatgggacagagtcgaacgtgccctcctctgcatactttccatcaccactaatgcataactggtcagttggcagttcaggcctgattctgcctcagtgaagttgaagcattggtcagacatttcaggtagagacagctagccccagggtgcctgtgccactctgtgacttagaagtgggatagtgtaacagatagaggggccaaagagaaaacactctggattcaggagcatcctaagtcatactctcattatgaacatgggattgactctctaagtagactgtgacaacatgtgatagcctaaggtggcccccaatattccctattcttgtggtttatatccttgtgcagtcccctcctactctgtaccaagttggtctgtgtgaccaagagagtaagggtgaagtgagggcatgtcacttctgagatcagagtggctcctcatctttgccactctctcctctctcaggttgctcacttttggggaaggaagccaccaagtgaagtggccttatggaagggtcacagtggtaaggaacttggccaatagtcatatggatagccaccctggacctggattctttggtctcattcaagtctgcagccccagccaacatcttaactctaacttcatgagaaaccctaaactggaagtcattcctggattcctgaccctcagaaactgggagataatgcctggtggcagtttttataaagctagcaaatttgggggtagtttgctacccagcaataattaactaatacaggtaggtataatcacatctatagtaggcagaatacgggcccatacaagatgtacacaccctaatcccctggagtctgtaaacataataccttacatggcaaaagggactaaactgatgtaattaacttaaggacacagagattggaagattatccttggtcacctgggtgtgcccaatctaagaacatgggtttttaagtcagataacctttccctgttgagttcagagtcagaggaagatgtgactatgaacaaatggttacagagatgcaacactgctgccttcaaagacagaagaaggaaaccatgagctgtggaagccagaaacagcatggaaattgattcttctttagaccctccagaaaggaatgcagccctgtcaacatcctgattttcagtccaatgagacatatatcagacttagaatgtatagaactctaagaaacttgtgttgttttaagccactcggtttgtggtaatttgttataacagcaacagaaactaataatacaccatcctccctgtattcttctactctgaactgccacagactttcagtatctattgggactaggtggtagaactagctgtgtgaccttgtgaaagtcacacaacttctctgagcctctgttctnnnnnnnnnnnnnnnnn

SEQ ID NO: 4 - DNA, 656 bp, Homo sapiens
ggccgccatttgggagaaaccggaggatttctaccctaatcgatttctggatgaccaaggacaactaattaaaaaagaaacctttattccttttgggatagggaagcgggtgtgtatgggagaacaactggcaaagatggaattattcctaatgtttgtgagcctaatgcagagtttcgcatttgctttacctgaggattctaagaagcccctcctgactggaagatttggtctaactttagccccacatccatttaatataactatttcaaggagatgaagagcatctccaagaagagatggtaaaaagatatataaatacatatccttctaagcagattcttcctactgcaaaggacagtgaatccagcaactcagtggatccaagctgggctcagaggtcggaaggagggtagagcacactgggaggtttcatcttggaggattcctcagcaggatacttcagccattttagtaatgcaggtctgtgatttgggggatagaaaacaaagtacctatgaaacgggatatctggattttacttgcagtggcttccaccgatgggccaatcttctcatttcttagtgcctcagacatcccatatgtaaaatgagagtaataaaacttggcttctctctaaaaaaaaaaaaaaaaaa

SEQ ID NO: 5 - DNA, 2256 bp, Homo sapiens
tgccgcttgccattcctcatatgacctcagagaacacagtgctccaagggtataccattcctaaaggcacattgatcttacccaacctgtggtcagtacatagagacccagccatttgggagaaaccggaggatttctaccctaatcgatttctggatgaccaaggacaactaattaaaaaagaaacctttattccttttgggatagggaagcgggtgtgtatgggagaacaactggcaaagatggaattattcctaatgtttgtgagcctaatgcagagtttcgcatttgctttacctgaggattctaagaagcccctcctgactggaagatttggtctaactttagccccacatccatttaatataactatttcaaggagatgaagagcatctccaagaagagatggtaaaaagatatataaatacatatccttctaagcagattcttcctactgcaaaggacagtgaatccagcaactcagtggatccaagctgggctcagaggtcggaaggagggtagagcacactgggaggtttcatcttggaggattcctcagcaggatacttcagccattttagtaatgcaggtctgtgatttgggggatagaaaacaaagtacctatgaaacgggatatctggattttacttgcagtggcttccaccgatgggccaatcttctcatttcttagtgcctcagacatcccatatgtaaaatgagagtaataaaacttggcttctctctacctctcagcactaatgatggtcaaatgccttacatcttttctgatatctctaaaatgctgttaagttctggagaagaacttcaggagaagaagatctatcagctggcttttaaagacctatgacaacatgaaagtggtgttcagcctggaatgctttgtcagagatgggtgtggatttaggttatactgggggagaacttttctcagcacagattctatgccagcttctttgggcttgttctgtcactatctttttgtttatgattttagtttttactttttgtagatgtgggatgaagtggactctgtcgtgtatattgaggaaaaaagaaattataattttaaaaaatcccttgtaggattattatctaaatttatatgtctaacttctactacaactacaggaacagtgagccttgctacttctttagtagcttcttggcagaattcctttctactgagttatttgcaaagatgcagctctacctttttacttaaggcctgaatggtgagcatggggattttgatactgggactcatcaggaaaggattctgctttcaaactatactgaacattcctgtcctagcgtccctgccaccaggcccaatgcatctgatccttgaatatactctcaaagaattcactctcgttttattaagagaactaaattgtttctaaatgtagatggtccctctggaaaagcagttttcagcaggggtggtaaccccttcagagggagtttggaaatgtgtgggtatgattcttggttatcataatgatgggggtgctactggccttctgctgccatgggaccaggatgctaaatgtcaaggtagtcctatacagtgaagaattgtcctgctcaagatgccaggatttcccccagtgagaacatgctctaaggaatgaccacccctttcttttattctcccacagtgctccatgtacagaagtaagcatagcagtcatatgagcaaccacattcctgaacctttcctcatgctggctctacacttaatcctttacttgtatgtttctgtaattcttacataaattctattaagagggtggcatactgtagtggatgaagctgaggcttatagtaggtaaggcacaaagttaaaaagtaacatcactgggtttcaaacctactggtctctgtgactaaagaacactttcagaaccacttcttgattctgccaccacttgatcccataacaggctaccccttggcctcatgctggagttgtgtgtgtctgtcttcatcccaggctgagctccttgaggtgaggatgttgtgctgtttgcctccctcacagtgccttggtcttagtggatgcccagttgtcttgtgaatgacttttaagaagtgtacttaagagaaaaatcctaccttatttgaataattacaagtcatgtttttgttgcttaaaggtgataaatcagtgtatattatttgttaatgtccattaaagccagtttttaaaaaaaaaaaaaaaaaaaaaaaaaa

SEQ ID NO: 6 - DNA, 44 bp, Artificial Sequence
  Description of Artificial Sequence synthetic oligdeoxyribonucleotide
agttgctggattcactgtccttaaggacagtgaatccagcaact

SEQ ID NO: 7 - DNA, 30 bp, Artificial Sequence
  Description of Artificial Sequence synthetic oligdeoxyribonucleotide
aagcagtggtaacaacgcagagtacgcggg

SEQ ID NO: 8 - DNA, 26 bp, Artificial Sequence
  Description of Artificial Sequence synthetic oligodeoxyribonucleotide
ggtttctcccaaatggctgggtctct

SEQ ID NO: 9 - DNA, 43 bp, Artificial Sequence
  Description of Artificial Sequence synthetic oligodeoxyribonucleotide
ctaatacgactcactatagggcaagcagtggtaacaaccagat

SEQ ID NO: 10 - DNA, 22 bp, Artificial Sequence
  Description of Artificial Sequence synthetic oligodeoxyribonucleotide
ctaatacgactcactatagggc

SEQ ID NO: 11 - DNA, 26 bp, Artificial Sequence
  Description of Artificial Sequence synthetic oligodeoxyribonucleotide
ccaccacagttagcctctgcacttcc

SEQ ID NO: 12 - DNA, 23 bp, Artificial Sequence
  Description of Artificial Sequence synthetic oligodeoxyribonucleotide
aagcagtggtaacaacgcagagt

SEQ ID NO: 13 - DNA, 1363 bp, Homo sapiens
tggtttctcccaaatggctgggtctcgatctcctaacctcatgatccgcccacctcagcctcccaaattgctgggattacagacgtgagccaccgtgcctggcccatgtacatgtatttttagaaatatatttgcctccttaaaatggaataattttcataattttaaaaattgctttttatcacttaaagtattatgagcatattttcatattattaaatattctcctgtaaagttcttaggagctgtataatatctgtttgtgagaatgtatcaatgtcaatttagtctatactttatttggtttgagttggatttttatgtttccactgtctagcacctgttccttggacaagaatacctgcttcttctgaggaattccttttttctgacctcagtcccttttacccttaactgtggttgaacggcagatgccatatctttacataaactcaccttcttggccacagtgataagggttgtgtttgcacattatggtcccgtctggagacaacaaaggaagttctctcattcaactcttcgtcattttgggttgggaaaacttagcttggagcccaagattattgaggagttcaaatatgtgaaagcagaaatgcaaaagcacggagaagaccccttctgccctttctccatcatcagcaatgccgtctctaacatcatttgctccttgtgctttggccagcgctttgattacactaatagtgagttcaagaaaatgcttggttttatgtcacgaggcctagaaatctgtctgaacagtcaagtcctcctggtcaacatatgcccttggcttttattaccttccctttggaccatttaaggaattaagacaaattgaaaaggatataaccagtttccttaaaaaaatcatcaaagaccatcaagagtctctggatagagagaaccctcaggacttcatagacatgtaccttctccacatggaagaggagaggaaaaataatagtaacagcagttttgatgaagagtacttattttatatcattggggatctctttattgctgggactgataccacaactaactctttgctctggtgcctgctgtatatgtcgctgaaccccgatgtacaagaaaaggttcatgaagaaattgaaagagtcattggcgccaaccgagctccttccctcacagacaaggcccagatgccctacacagaagccaccatcatggaagtgcagaggctaactgtggtggtgccgcttgccattcctcatatgacctcagagaacacagtgctccaagggtataccattcctaaaggcacattgatcttacccaacctgtggtcagtacatagagacccagccatttgggagaaacc

SEQ ID NO: 14 - DNA, 25 bp, Artificial Sequence
  Description of Artificial Sequence synthetic deoxyribonucleotide
gaggtcatatgaggaatggcaagcg

SEQ ID NO: 15 - DNA, 24 bp, Artificial Sequence
  Description of Artificial Sequence synthetic deoxyribonucleotide
tgcccttggctttattaccttccc

SEQ ID NO: 16 - DNA, 3544 bp, Homo sapiens
  CDS (40)..(1671)
ctcaagcaaggggaacccgaggccgccggcgcccggaccatgtcgtctccggggccgtcgcagccgccggccgaggacccgccctggcccgcgcgcctcctgcgtgcgcctctggggctgctgcggctggaccccagcgggggcgcgctgctgctatgcggcctcgtagcgctgctgggctggagctggctgcggaggcgccgggcgcggggcatcccgcccgggcccacgccctggcctctggtgggcaacttcggtcacgtgctgctgcctcccttcctccggcggcggagctggctgagcagcaggaccagggccgcagggattgatccctcggtcataggcccgcaggtgctcctggctcacctagcccgcgtgtacggcagcatcttcagcttctttatcggccactacctggtggtggtcctcagcgacttccacagcgtgcgcgaggcgctggtgcagcaggccgaggtcttcagcgaccgcccgcgggtgccgctcatctccatcgtgaccaaggagaagggggttgtgtttgcacattatggtcccgtctggagacaacaaaggaagttctctcattcaactcttcgtcattttgggttgggaaaacttagcttggagcccaagattattgaggagttcaaatatgtgaaagcagaaatgcaaaagcacggagaagaccccttctgccctttctccatcatcagcaatgccgtctctaacatcatttgctccttgtgctttggccagcgctttgattacactaatagtgagttcaagaaaatgcttggttttatgtcacgaggcctagaaatctgtctgaacagtcaagtcctcctggtcaacatatgcccttggctttattaccttccctttggaccatttaaggaattaagacaaattgaaaaggatataaccagtttccttaaaaaaatcatcaaagaccatcaagagtctctggatagagagaaccctcaggacttcatagacatgtaccttctccacatggaagaggagaggaaaaataatagtaacagcagttttgatgaagagtacttattttatatcattggggatctctttattgctgggactgataccacaactaactctttgctctggtgcctgctgtatatgtcgctgaaccccgatgtacaagaaaaggttcatgaagaaattgaaagagtcattggcgccaaccgagctccttccctcacagacaaggcccagatgccctacacagaagccaccatcatggaagtgcagaggctaactgtggtggtgccgcttgccattcctcatatgacctcagagaacacagtgctccaagggtataccattcctaaaggcacattgatcttacccaacctgtggtcagtacatagagacccagccatttgggagaaaccggaggatttctaccctaatcgatttctggatgaccaaggacaactaattaaaaaagaaacctttattccttttgggatagggaagcgggtgtgtatgggagaacaactggcaaagatggaattattcctaatgtttgtgagcctaatgcagagtttcgcatttgctttacctgaggattctaagaagcccctcctgactggaagatttggtctaactttagccccacatccatttaatataactatttcaaggagatgaagagcatctccaagaagagatggtaaaaagatatataaatacatatccttctaagcagattcttcctactgcaaaggacagtgaatccagcaactcagtggatccaagctgggctcagaggtcggaaggagggtagagcacactgggaggtttcatcttggaggattcctcagcaggatacttcagccattttagtaatgcaggtctgtgatttgggggatagaaaacaaagtacctatgaaacgggatatctggattttacttgcagtggcttccaccgatgggccaatcttctcatttcttagtgcctcagacatcccatatgtaaaatgagagtaataaaacttggcttctctctacctctcagcactaatgatggtcaaatgccttacatcttttctgatatctctaaaatgctgttaagttctggagaagaacttcaggagaagaagatctatcagctggcttttaaagacctatgacaacatgaaagtggtgttcagcctggaatgctttgtcagagatgggtgtggatttaggttatactgggggagaacttttctcagcacagattctatgccagcttctttgggcttgttctgtcactatctttttgtttatgattttagtttttactttttgtagatgtgggatgaagtggactctgtcgtgtatattgaggaaaaaagaaattataattttaaaaaatcccttgtaggattattatctaaatttatatgtctaacttctactacaactacaggaacagtgagccttgctacttctttagtagcttcttggcagaattcctttctactgagttatttgcaaagatgcagctctacctttttacttaaggcctgaatggtgagcatggggattttgatactgggactcatcaggaaaggattctgctttcaaactatactgaacattcctgtcctagcgtccctgccaccaggcccaatgcatctgatccttgaatatactctcaaagaattcactctcgttttattaagagaactaaattgtttctaaatgtagatggtccctctggaaaagcagttttcagcaggggtggtaaccccttcagagggagtttggaaatgtgtgggtatgattcttggttatcataatgatgggggtgctactggccttctgctgccatgggaccaggatgctaaatgtcaaggtagtcctatacagtgaagaattgtcctgctcaagatgccaggatttcccccagtgagaacatgctctaaggaatgaccacccctttcttttattctcccacagtgctccatgtacagaagtaagcatagcagtcatatgagcaaccacattcctgaacctttcctcatgctggctctacacttaatcctttacttgtatgtttctgtaattcttacataaattctattaagagggtggcatactgtagtggatgaagctgaggcttatagtaggtaaggcacaaagttaaaaagtaacatcactgggtttcaaacctactggtctctgtgactaaagaacactttcagaaccacttcttgattctgccaccacttgatcccataacaggctaccccttggcctcatgctggagttgtgtgtgtctgtcttcatcccaggctgagctccttgaggtgaggatgttgtgctgtttgcctccctcacagtgccttggtcttagtggatgcccagttgtcttgtgaatgacttttaagaagtgtacttaagagaaaaatcctaccttatttgaataattacaagtcatgtttttgttgcttaaaggtgataaatcagtgtatattatttgttaatgtccattaaagccagtttttaaaaaaaaaaaaaaaaaaaaaaaaaa

SEQ ID NO: 17 - protein, 544 aa, Homo sapiens
MSSPGPSQPPAEDPPWPARLLRAPLGLLRLDPSGGALLLCGLVALLGWSWLRRRRARGIPPGPTPWPLVGNFGHVLLPPFLRRRSWLSSRTRAAGIDPSVIGPQVLLAHLARVYGSIFSFFIGHYLVVVLSDFHSVREALVQQAEVFSDRPRVPLISIVTKEKGVVFAHYGPVWRQQRKFSHSTLRHFGLGKLSLEPKIIEEFKYVKAEMQKHGEDPFCPFSIISNAVSNIICSLCFGQRFDYTNSEFKKMLGFMSRGLEICLNSQVLLVNICPWLYYLPFGPFKELRQIEKDITSFLKKIIKDHQESLDRENPQDFIDMYLLHMEEERKNNSNSSFDEEYLFYIIGDLFIAGTDTTTNSLLWCLLYMSLNPDVQEKVHEEIERVIGANRAPSLTDKAQMPYTEATIMEVQRLTVVVPLAIPHMTSENTVLQGYTIPKGTLILPNLWSVHRDPAIWEKPEDFYPNRFLDDQGQLIKKETFIPFGIGKRVCMGEQLAKMELFLMFVSLMQSFAFALPEDSKKPLLTGRFGLTLAPHPFNITISRR

SEQ ID NO: 18 - DNA, 711 bp, Homo sapiens
tttttttagagagaagccaagttcttatattctcattttacatatgggatgtgtgaggcactaataaatgagaagattggcccatcggtggaagccactgcaagtaaaatccagatatcccgtttcataggtactttgttttctatcccccaaatcacagacctgcattactaaaatggctgaagtatcctgctgaggaatcctccaagatgaaacctcccagtgtgctctaccctccttccgacctctgagcccagcttggatccactgagttgctggattcactgtcctttgcagtaggaagaatctgcttataaggatatgtatttatatatctttttaccatctcttcttggagatgctcttcatctccttgaaatagttatattaaatggatgtggggctaaagttagaccaaatcttccagtcaggaggggcttcttataatcctcaggtaaagcaaatgccatactctgcattaggctcacacacattaggaataattccatctttgccagttgttctcccatacacactccgcttccctatcccaaaaggaataaaggctgctgtcttaattagttgtcccttgtcatccagaaatcgattatggtagaaatcctccggtttcttccaaatggctgggtctctatgtactgaccacatgctgggtgagatacatgcgcctcttggaaaggtatacccttcgag

SEQ ID NO: 19 - DNA, 463 bp, Homo sapiens
tttttttttttttgagagaagccaagttttattactctcattttacatatgggatgtctgaggcactaagaaatgagaagattggcccatcggtggaagccactgcaagtaaaatccagatatcccgtttcataggtactttgttttctatcccccaaatcacagacctgcattactaaaatggctgaagtatcctgctgaggaatcctccaagatgaaacctcccagtgtgctctaccctccttccgacctctgagcccagcttggatccactgagttgctggattcactgtcctttgcagtaggaagaatctgcttagaaggatatgtatttatatatctttttaccatctcttcttggagatgctcttcatctccttgaaatagttattaaatggatgtggggctaaagttagaccaaatcttccagtcaggaggggcttcttagaatcctcaggtaaag

SEQ ID NO: 20 - DNA, 551 bp, Homo sapiens
cctcaatatacacgacagagtccacttcatcccacatctacaaaaagtaaaaactaaaatcataaacaaaaagatagtgacagaacaagcccaaagaagctggcatagaatctgtgctgagaaaagttctcccccagtataacctaaatccacacccatctctgacaaagcattccaggctgaacaccactttcatgttgtcataggtctttaaaagccagctgatagatcttcttctcctgaagttcttctccagaacttaacagcattttagagatatcagaaaagatgtaaggcatttgaccatcattagtgctgagaggtagagagaagccaagttttattactctcattttacatatgggatgtctgaggcactaagaaatgagaagattggcccatcggtggaagccactgcaagtaaaatccagatatcccgtttcataggtactttgttttctatcccccaaatcacagacctgcattactaaaatggctgaagtatcctgctgaggaatcctccaagatgaaaacctccagtgtgctctacc

SEQ ID NO: 21 - DNA, 493 bp, Homo sapiens
cctcaatatacacgacagagtccacttcatcccacatctacaaaaagtaaaaactaaaatcataaacaaaaagatagtgacagaacaagcccaaagaagctggcatagaatctgtgctgagaaaagttctcccccagtataacctaaatccacacccatctctgacaaagcattccaggctgaacaccactttcatgttgtcataggtctttaaaagccagctgatagatcttcttctcctgaagttcttctccagaacttaacagcattttagagatatcagaaaagatgtaaggcatttgaccatcattagtgctgagaggtagagagaagccaagttttattactctcattttacatatgggatgtctgaggcactaagaaatgagaagattggcccatcggtggaagccactgcaagtaaaatccagatatcccgtttcataggtactttgttttctatcccccaaatcacagacctgcattactaaaa

SEQ ID NO: 22 - DNA, 499 bp, Homo sapiens
aaaaactggctttaatggacattaacaaataatatacactgatttatcacctttaagcaacaaaaacatgacttgtaattattcaaataaggtaggatttttctcttaagtacacgtcttaaaagtcattcacaagacgactgggcatccactaagaccaaggcactgtgagggaggcaaacagcacaacatcctcacctcaaggagctcagcctgggatgaagacagacacacacaactccagcatgaggccaaggggtagcctgttatgggatcaagtggtggcagaatcaagaagtggttctgaaagtgttctttagtcacagagaccagtaggtttgaaacccagtgatgttactttttaactttgtgccttacctactataagcctcagcttcatccactacagtatgccaccctcttaatagaatttatgtaagaattacagaaacatacaagtaaaggattaagtgtagagccagcatgaggaaaggttcag

SEQ ID NO: 23 - DNA, 493 bp, Homo sapiens
tttttaaaaactggctttaatggacattaacaaataatatacactgatttatcacctttaagcaacaaaaacatgacttgtaattattcaaataaggtaggatttttctcttaagtacacttcttaaaagtcattcacaagacaactgggcatccactaagaccaaggcactgtgagggaggcaaacagcacaacatcctcacctcaaggagctcagcctgggatgaagacagacacacacaactccagcatgaggccaaggggtagcctgttatgggatcaagtggtggcagaatcaagaagtggttctgaaagtgttctttagtcacagagaccagtaggtttgaaacccagtgatgttactttttaactttgtgccttacctactataagcctcagcttcatccactacagtatgccaccctcttaatagaatttatgtaagaattacagaaacatacaagtaaaggattaagtgtagagccagcatgag

SEQ ID NO: 24 - DNA, 488 bp, Homo sapiens
tttttttttaaaaactggctttaatggacattaacaaataatatacactgatttatcacctttaagcaacaaaaacatgacttgtaattattcaaataaggtaggatttttctcttaagtacacttcttaaaagtcattcacaagacaactgggcatccactaagaccaaggcactgtgagggaggcaaacagcacaacatcctcacctcaaggagctcagcctgggatgaagacagacacacacaactccagcatgaggccaaggggtagcctgttatgggatcaagtggtggcagaatcaagaagtggttctgaaagtgttctttagtcacagagaccagtaggtttgaaacccagtgatgttactttttaactttgtgccttacctactataagcctcagcttcatccactacagtatgccaccctcttaatagaatttatgtaagaattacagaaacatacaagtaaaggattaagtgtagagc

SEQ ID NO: 25 - DNA, 487 bp, Homo sapiens
aaaaactggctttaatggacattaacaaataatatacactgatttatcacctttaagcaacaaaaacatgacttgtaattattcaaataaggtaggatttttctcttaagtacacgtcttaaaagtcattcacaagacgactgggcatccactaagaccaaggcactgtgagggaggcaaacagcacaacatcctcacctcaaggagctcagcctgggatgaagacagacacacacaactccagcatgaggccaaggggtagcctgttatgggatcaagtggtggcagaatcaagaagtggttctgaaagtgttctttagtcacagagaccagtaggtttgaaacccagtgatgttactttttaactttgtgccttacctactataagcctcagcttcatccactacagtatgccaccctcttaatagaatttatgtaagaattacagaaacatacaagtaaaggattaagtgtagagccagcatga

SEQ ID NO: 26 - DNA, 485 bp, Homo sapiens
aaaactggctttaatggacattaacaaataatatacactgatttatcacctttaagcaacaaaaacatgacttgtaattattcaaataaggtaggatttttctcttaagtacacgtcttaaaagtcattcacaagacgactgggcatccactaagaccaaggcactgtgagggaggcaaacagcacaacatcctcacctcaaggagctcagcctgggatgaagacagacacacacaactccagcatgaggccaaggggtagcctgttatgggatcaagtggtggcagaatcaagaagtggttctgaaagtgttctttagtcacagagaccagtaggtttgaaacccagtgatgttactttttaactttgtgccttacctactataagcctcagcttcatccactacagtatgccaccctcttaatagaatttatgtaagaattacagaaacatacaagtaaaggattaagtgtagagccagcatg

SEQ ID NO: 27 - DNA, 483 bp, Homo sapiens
ttttaaaaactggctttaatggacattaacaaataatatacactgatttatcacctttaagcaacaaaaacatgacttgtaattattcaaataaggtaggatttttctcttaagtacacgtcttaaaagtcattcacaagacgactgggcatccactaagaccaaggcactgtgagggaggcaaacagcacaacatcctcacctcaaggagctcagcctgggatgaagacagacacacacaactccagcatgaggccaaggggtagcctgttatgggatcaagtggtggcagaatcaagaagtggttctgaaagtgttctttagtcacagagaccagtaggtttgaaacccagtgatgttactttttaactttgtgccttacctactataagcctcagcttcatccactacagtatgccaccctcttaatagaatttatgtaagaattacagaaacatacaagtaaaggattaagtgtagagc

SEQ ID NO: 28 - DNA, 469 bp, Homo sapiens
tttttaaaaactggctttaatggacattaacaaataatatacactgatttatcacctttaagcaacaaaaacatgacttgtaattattcaaataaggtaggatttttctcttaagtacacgtcttaaaagtcattcacaagacgactgggcatccactaagaccaaggcactgtgagggaggcaaacagcacaacatcctcacctcaaggagctcagcctgggatgaagacagacacacacaactccagcatgaggccaaggggtagcctgttatgggatcaagtggtggcagaatcaagaagtggttctgaaagtgttctttagtcacagagaccagtaggtttgaaacccagtgatgttactttttaactttgtgccttacctactataagcctcagcttcatccactacagtatgccaccctcttaatagaatttatgtaagaattacagaaacatacaagtaaag

SEQ ID NO: 29 - DNA, 463 bp, Homo sapiens
  unsure (461): n can be any nucleic acid
aaaaactggctttaatggacattaacaaataatatacactgatttatcacctttaagcaacaaaaacatgacttgtaattattcaaataaggtaggatttttctcttaagtacacgtcttaaaagtcattcacaagacgactgggcatccactaagaccaaggcactgtgagggaggcaaacagcacaacatcctcacctcaaggagctcagcctgggatgaagacagacacacacaactccagcatgaggccaaggggtagcctgttatgggatcaagtggtggcagaatcaagaagtggttctgaaagtgttctttagtcacagagaccagtaggtttgaaacccagtgatgttactttttaactttgtgccttacctactataagcctcagcttcatccactacagtatgccaccctcttaatagaatttatgtaagaattaccgaaacatacaagtnaa

SEQ ID NO: 30 - DNA, 452 bp, Homo sapiens
aaaaactggctttaatggacattaacaaataatatacactgatttatcacctttaagcaacaaaaacatgacttgtaattattcaaataaggtaggatttttctcttaagtacacgtcttaaaagtcattcacaagacgactgggcatccactaagaccaaggcactgtgagggaggcaaacagcacaacatcctcacctcaaggagctcagcctgggatgaagacagacacacacaactccagcatgaggccaaggggtagcctgttatgggatcaagtggtggcagaatcaagaagtggttctgaaagtgttctttagtcacagagaccagtaggtttgaaacccagtgatgttactttttaactttgtgccttacctactataagcctcagcttcatccactacagtatgccaccctcttaatagaatttatgtaagaattacagaaac

SEQ ID NO: 31 - DNA, 439 bp, Homo sapiens
gggaccaggatgctaaatgtcaaggtagtcctatacagtgaagaattgtcctgctcaagatgccaggatttccccagtgagaacatgctctaaggaatgaccacccctttcttttattctcccacagtgctccatgtacagaagtaagcatagcagtcatatgagcaaccacattcctgaacctttcctcatgctggctctacacttaatcctttacttgtatgtttctgtaattcttacataaattctattaagagggtggcatactgtagtggatgaagctgaggcttatagtaggtaaggcacaaagttaaaaagtaacatcactgggtttcaaacctactggtctctgtgactaaagaacactttcagaaccacttcttgattctgccaccacttgatcccataacaggctaccccttggcctcatgctggagtt

SEQ ID NO: 32 - DNA, 576 bp, Homo sapiens
  unsure (501): n can be any nucleic acid
ttttttaaaaactggctttaatggacattaacaaataatatacactgatttatcacctttaagcaacaaaaacatgacttgtaattattcaaataaggtaggatttttctcttaagtacacttcttaaaagtcattcacaagacaactgggcatccactaagaccaaggcactgtgagggaggcaaacagcacaacatcctcacctcaaggagctcagcctgggatgaagacagacacacaactccagcatgaggccaaggggtagcctgttatgggatcaagtggtggcagaatcaagaagtggttctgaaagtgttctttagtcacagagaccagtaggtttgaaacccagtgatgttactttttaactttgtgccttacctactataagcgtcagcttcatccactacagtatgccacctcttaatagatttatgtaagattacagaacatacagtaaggattagtgtagagcagcatgaggcaaggtcaggatgtgntgctcaatgatgctatcttactctgtcatggacactgtggagatnaagaaggggtgcatcttagacatgtccact

SEQ ID NO: 33 - DNA, 495 bp, Homo sapiens
ccagagggaccatctacatttagaaacaatttagttctcttaataaaaagagagtgaattctttgagagtatattcaaggatcagatgcattgggcctggtggcagggacgctaggacaggaatgttcagtatagtttgaaagcagaatcctttcctgatgagtcccagtatcaaaatccccatgctcaccattcaggccttaagtaaaaaggtagagctgcatctttgcaaataactcagtagaaaggaattctgccaagaagctactaaagaagtagcaaggctcactgttcctgtagttgtagtagaagttagacatataaatttagataataatcctacaagggattttttaaaattataatttcttttttcctcaatatacacgacagagtccacttcatcccacatctacaaaaagtaaaaactaaaatcataaacaaaaagatagtgacagaacaagcccaaagaagctggcatagaatctgtgctga

SEQ ID NO: 34 - DNA, 443 bp, Homo sapiens
  unsure (386): n can be any nucleic acid
ctatacagtgaagaattgtcctgctcaagatgccaggatttcccccagtgagaacatgctctaaggaatgaccacccctttcttttattctcccacagtgctccatgtacagaagtaagcatagcagtcatatgagcaaccacattcctgaacctttcctcatgctggctctacacttaatcctttacttgtatgtttctgtaattcttacataaattctattaagagggtggcatactgtagtggatgaagctgaggcttatagtaggtaaggcacaaagttaaaaagtaacatcactgggtttcaaacctactggtctctgtgactaaagaacactttcagaaccacttcttgattctgccaccacttgatcccataacagggntaccccttgggcctcatggctgggagttgtgtgtgtctgtctttcatcccngggttg

SEQ ID NO: 35 - DNA, 395 bp, Homo sapiens
taaaaactggctttaatggacattaacaaataatatacactgatttatcacctttaagcaacaaaaacatgacttgtaattattcaaataaggtaggatttttctcttaagtacacttcttaaaagtcattcacaagacaactgggcatccactaagaccaaggcactgtgggggaggcaaacagcacaacatcctcacctcaaggagctcagcctgggatgaagacagacacacacaactccagcatgaggccaaggggtagcctgttatgggatcaagtggtggcagaatcaagaagtggttctgaaagtgttctttagtcacagagaccagtaggtttgaaacccagtgatgttactttttaactttgtgccttacctactataagcctcag

SEQ ID NO: 36 - DNA, 632 bp, Homo sapiens
  unsure (561): n can be any nucleic acid
gcgatgaagctgaggcttatagtaggtaaggcacaaagttaaaaagtaacatcactgggtttcaaacctactggtctctgtgactaaagaacactttcagaaccacttcttgattctgccaccacttgatcccataacaggctaccccttggcctcatgctggagttgtgtgtgtctgtcttcatcccaggctgagctccttgaggtgaggatgttgtgctgtttgcctccctcacagtgccttggtcttaagtggatgcccagttgtcttgtgaatgacttttaaagaagtgtacttaagaagaaaaatcctaccttatttgaataattacaagtcatgtttttgttgcttaaaggtgataaatcagtgtatattatttgttaatgtccattaaagccagtttttaaaaaaatcactgggacttttgtggtttaccatttaaaacaaccattttaaacaaacttagaacgatgccttagctggtttataggtaacagaatgtaaactccaccacctggagactaggccatatccagggacctcacactgactttcctaangagagaagcagctacgccgttccataccggatagagaaganggcaagaaaagatccagagtngctgttacn

SEQ ID NO: 37 - DNA, 395 bp, Homo sapiens
tttttttttaaaaactggctttaatggacattaacaaataatatacactgatttatcacctttaagcaacaaaaacatgacttgtaattattcaaataaggtaggatttttctcttaagtacacgtcttaaaagtcattcacaagacgactgggcatccactaagaccaaggcactgtgagggaggcaaacagcacaacatcctcacctcaaggagctcagcctgggatgaagacagacacacacaactccagcatgaggccaaggggtagcctgttatgggatcaagtggtggcagaatcaagaagtggttctgaaagtgttctttagtcacagagaccagtaggtttgaaacccagtgatgttactttttaactttgtgccttacctactata

SEQ ID NO: 38 - DNA, 428 bp, Homo sapiens
ttttttttttttttttttttttttttttttttttttttttttttttttttaaaaactggctttaatggacattaacaaataatatacactgatttatcacctttaagcaacaaaaacatgacttgtaattattcaaataaggtaggatttttctcttaagtacacgtcttaaaagtcattcacaagacgactgggcatccactaagaccaaggcactgtgagggaggcaaacagcacaacatcctcacctcaaggagctcagcctgggatgaagacagacacacacaactccagcatgaggccaaggggtagcctgttatgggatcaagtggtggcagaatcaagaagtggttctgaaagtgttctttagtcacagagaccagtaggtttgaaacccagtgatgttactttttaactttgtgccttac

SEQ ID NO: 39 - DNA, 427 bp, Homo sapiens
ctcaatatacacgacagagtccacttcatcccacatctacaaaaagtaaaaactaaaatcataaacaaaaagatagtgacagaacaagcccaaagaagctggcatagaatctgtgctgagaaaagttctcccccagtataacctaaatccacacccatctctgacaaagcattccaggctgaacaccactttcatgttgtcataggtctttaaaagccagctgatagatcttcttctcctgaagttcttctccagaacttaacagcattttagagatatcagaaaagatgtaaggcatttgaccatcattagtgctgagaggtagagagaagccaagttttattactctcattttacatatgggatgtctgaggcactaagaaatgagaagattggcccatcggtggaagccactgcaagtaaaatc

SEQ ID NO: 40 - DNA, 442 bp, Homo sapiens
  unsure (404): n can be any nucleic acid
tttttttttttttttttttttttttttttttttaaaaactggctttaatggacattaacaaataatatacactgatttatcacctttaagcaacaaaaacatgacttgtaattattcaaataagggaggattttttttttaagtccactttttaaaagtcattcacaaaacaactgggcatccactaaaaccaaggcactgggagggaggcaaacagcacaacatcctcacctcaaggagctcagcctgggatgaaaacagacacacacaactccagcatgaggccaaggggtagcctgttatgggatcaagtgggggcaaaatcaaaaaggggttctgaaagtgttctttagtcacagagaccagtaggtttgaaacccagggatgttactttttaactttgngccttacctactataagcctcagcttcatccactacag

SEQ ID NO: 41 - DNA, 385 bp, Homo sapiens
  unsure (95): n can be any nucleic acid
aagaattcactctcgttttattaagagaactaaattgtttctaaatgtagatggtccctctggaaaagcagttttcagcaggggtggtaaccttncagagggagtttggaaatgtgtgggtatgattcttggttatcataatgatgggggtgctactggccttctgctgccatgggaccaggatgctaaatgtcaaggtagtcctatacagtgaagaattgtcctgctcaagatgccaggatttcccccagtgagaacatgctctaaggaatgaccacccctttcttttattctcccacagtgctccatgtacagaagtaaggcatagcagtcatatgaggcaaccacattcctgaacctttnctcatgctgggctctacattta

SEQ ID NO: 42 - DNA, 331 bp, Homo sapiens
tttttttttaaaaactggctttaatggacattaacaaataatatacactgatttatcacctttaagcaacaaaaacatgacttgtaattattcaaataaggtaggatttttctcttaagtacacttcttaaaagtcattcacaagacaactgggcatccactaagaccaaggcactgtgagggaggcaaacagcacaacatcctcacctcaaggagctcagcctgggatgaagacagacacacacaactccagcatgaggccaaggggtagcctgttatgggatcaagtggtggcagaatcaagaagtggttctgaaagtgttctttagtc

SEQ ID NO: 43 - DNA, 336 bp, Homo sapiens
tttttttttttttttttaaaaactggctttaatggacattaacaaataatatacactgatttatcacctttaagcaacaaaaacatgacttgtaattattcaaataaggtaggatttttctcttaagtacacgtcttaaaagtcattcacaagacgactgggcatccactaagaccaaggcactgtgagggaggcaaacagcacaacatcctcacctcaaggagctcagcctgggatgaagacagacacacacaactccagcatgaggccaaggggtagcctgttatgggatcaagtggtggcagaatcaagaagtggttctgaaagtgttcttta

SEQ ID NO: 44 - DNA, 271 bp, Homo sapiens
cataacaggctaccccttggcctcatgctggagttgtgtgtgtctgtcttcatcccaggctgagctccttgaggtgaggatgttgtgctgtttgcctccctcacagtgccttggtcttagtggatgcccagttgtcttgtgaatgacttttaagaagtgtacttaagagaaaaatcctaccttatttgaataattacaagtcatgtttttgttgcttaaaggtgataaatcagtgtatattatttgttaatgtccattaaagccagttttt

SEQ ID NO: 45 - DNA, 452 bp, Homo sapiens
gccccccgtggtgccgcttttaaagacttatgacaacatgaaagtggtgttcagcctggaatgctttgtcagagatgggtgtggatttaggttatactgggggagaacttttctcagcacagattctatgccagcttctttgggcttgttctgtcactatctttttgtttatgattttagtttttactttttgtagatgtgggatgaagtggactctgtcgtgtatattgaggaaaaaagaaattataattttaaaaaatcccttgtaggattattatctaaatttatatgtctaacttctactacaactacaggaacagtgagccttgctacttctttagtagcttcttggcagaattcctttctactgagttatttgcaaagatgcagctctacctttttacttaaggcctgaatggtgagcatggggattttgatactgggactcatca

SEQ ID NO: 46 - DNA, 284 bp, Homo sapiens
ttttttttttttttttttttttttttttttaaaaactggctttaatggacattaacaaataatatacactgatttatcacctttaagcaacaaaaacatgacttgtaattattcaaataaggtaggatttttctcttaagtacacttcttaaaagtcattcacaagacaactgggcatccactaagaccaaggcactgtgagggaggcaaacagcacaacatcctcacctcaaggagctcagcctgggatgaagacagacacacacaactccagcatgaggcca

SEQ ID NO: 47 - DNA, 277 bp, Homo sapiens
  unsure (187): n can be any nucleic acid
gatcccataacaggctaccccttggcctcatgctggagttgtgtgtgtctgtcttcatcccaggctgagctccttgaggtgaggatgttgtgctgtttgcctccctcacagtgccttggtcttagtggatgcccagttgtcttgtgaatgacttttaagaagtgtacttaagagaaaaatcctaccntatnnnnngtaattacaagtcatgtttttgtngcttaaaggtgataaatcagtgtatatnatnngntaatgtccattaaagccagttttt

SEQ ID NO: 48 - DNA, 446 bp, Homo sapiens
  unsure (361): n can be any nucleic acid
gacttttaagaagtgtacttaagagaaaaatcctaccttatttgaataattacaagtcatgtttttgttgcttaaaggtgataaatcagtgtatattatttgttaatgtccattaaagccagtttttaaaaaaatcactggacttttgtgtttaccatttaaaacaaccattttaaacaaacttagaacgatgccttagctgtttataggtaacagaatgtaaactcccaccacctggagactaggccatatccagggacctcacactgactttcctaaaggagagaagcagctacgctgttcatactgatagagaagagggcaagaaaagatccagagtggctgttactttgaaaattanccganagatgaatntgacttaaaacactctgtttggctcctataactgatgacctctttntctgtttncagataatggagcacca

SEQ ID NO: 49 - DNA, 308 bp, Homo sapiens
  unsure (306): n can be any nucleic acid
aagacgtgtacttaagagaaaaatcctaccttatttgaataattacaagtcatgtttttgttgcttaaaggtgataaatcagtgtatattatttgttaatgtccattaaagccagtttttaaaaaaatcactggacttttgtgtttaccatttaaaacaaccattttaaacaaacttagaacgatgccttagctgtttataggtaacagaatgtaaactcccaccacctggagactaggccatatccagggacctcacactgactttcctaaaggagagaagcagctacgctgtgtcatactgatnga

SEQ ID NO: 50 - DNA, 361 bp, Homo sapiens
acgtgtacttaagagaaaaatcctaccttatttgaataattacaagtcatgtttttgttgcttaaaggtgataaatcagtgtatattatttgttaatgtccattaaagccagtttttaaaaaaatcactggacttttgtgtttaccatttaaaacaaccattttaaacaaacttagaacgatgccttagctgtttataggtaacagaatgtaaactcccaccacctggagactaggccatatccagggacctcacactgactttcctaaaggagagaagcagctacgctgttcatactgatagagaagagggcaagaaaagatccagagtggctgttactttgaaaattaaacgaaagatg

SEQ ID NO: 51 - DNA, 164 bp, Homo sapiens
cctcaatatacacgacagagtccacttcatcccacatctacaaaaagtaaaaactaaaatcataaacaaaaagatagtgacagaacaagcccaaagaagctggcatagaatctgtgctgagaaaagttctcccccagtataacctaaatccacacccatctctg

SEQ ID NO: 52 - DNA, 350 bp, Homo sapiens
  unsure (216): n can be any nucleic acid
aatcagtgtatattatttgttaatgtccattaaagccagtttttaaaaaaatcactggacttttgtgtttaccatttaaaacaaccattttaaacaaacttagaacgatgccttagctgtttataggtaacagaatgtaaactcccaccacctggagactaggccatatccagggacctcacactgactttcctaaaggagagaagcagctacgcntttcatactgatagagaagagggcaagaaaagatccagagtggctgttactttgaaaattaaacgaaagatgaaattctgaacttaaaacacttctgtttggccttctataactgatcacccttctttgctctg

SEQ ID NO: 53 - protein, 497 aa, Fundulus heteroclitus
MWLYNFLLVLDLKAILLFIFSFLLIADFLRNRKPANFPPGPKALPFVGNMLNLDSQHPHIFFSKLADIYGNVFSFRLGKESMVVVSGHKLVKEAIVTQGENFVDRPPNAIAERFYTEPSGGLFFNNGEIWKRQRRFALSTLRTFGLGKNTLELSICEEIRHLQEEIENEKGKPFSPAGLFNNAVSNIICQLVMGRRFDYHDQSFQTMLKYMSEALWLEGSIWGQLYQAFPQVMKYIPGPHNKLFSNFTAIKELLQEEIEKHKKDLDHSNPRDYIDTFLIKMENQQEAELGFTERNLAFCSLDLFLAGTETTATTLLWALLFLIKYPEVQEKVHAEIDRVIGQTRLPSMADRPNLPYTDAVIHEIQRMSNIVPLNGLRVASKDTTLGGYFIPKGTAVMPMLTSVLFDKTEWETPDTFNPGHFLDANGKFVKKEAFLPFSAGKRVCLGEGLAKMELFLFLVALLQKFSFSAPEGVELSTEGITGITLVPHPYKVSAKAR

SEQ ID NO: 54 - protein, 497 aa, Fundulus heteroclitus
MWFYNLLLSLDVKGLFLFIFLFLLIADFYKSRKPANFPPGPKALPFVGNFFSLDSKHPHVYFQKLAEIYGNVFSFRLGRDSIVFLNGYKAVREALVTQAENFVDRPFNAITDRFYTEPSAGIFMSNGEKWKKQRRFALSTLRNFGLGKNSLEQSVSEEIQHLQEEMEIEKGKPFNPSGLFTNAVSNIICQLVMGKRYDYTDHRFQMMLRCMSEAVLLEGNVWGQLYMAFPSVMRYMPGPHNKIFSHFSSVEQFLYEEVEQHKKDLDRDNPRDYIDTFLIEMENHKESDLGFTEANLVYCAIDLFLAGTETTATTLLWALVFLVKYPEVQEKVQAEIDSVIEQARLPSMADRSSMPYTDAVIHEIQRIGNILPLNGMRVAAKDTTLGGYFIPKGTSLMPVLTSVLFDKAEWACPDTFNPGHFLDDNGKFVKRDAFLPFSAGKRACIGESLAKMELFLFLVALLQKFTFSVPEGVELSTEGITGTTRVPHPYKVSAKIR

SEQ ID NO: 55 - DNA, 2542 bp, Homo sapiens
  unsure (855): n can be any nucleic acid
gaattcagcccagatttcctatctgccccgcacagctattaacctagtgaaaatgagagatttaggaaaacactacacgtgcagttacatcctgtacaatataaaagggtggctgttggagttgttaaattagtcacagcttcaactattttaaaatcatttatgaatttaaattgcttgtatgggatgatgtatatagtggctactgtttcagagagcaaagccctagaatcagatgacatccagaatgtcatgttgaagatcgtatctgcaaacaagtagtttaaatttttaaaaacttctcttaattcaaaaattcaaaaaaaatgagaaaagtaatctcacaagtagtgggttaattatagaagatgagaggtgtgggaggttttttttttttgtttggggtgcataagcttaaaataacatgaaaagcatgtcaaaaagaatatgagaacagagtggacttgaaatttaacatatcctactggctcacttgttatagaaaaataaaatggatctgtatttatttgaggactcctatgctttctttgatccatttttataggtacagcgtggtgttcaaacttttaatgtatggagaaaaatgaaacggctagaaaaatggcccagtacagacaattctatgctcaatccaacttgaacagaaactacaggatttggcctaaacaagactgaaagggaagcccttatcaggaaagccattatctgagaagataaaataaagtaatactttgtttttaaagtattatacaattcatatctaatatttcaacctcagcctttcaagtgttgtctaacaccaataataatattgccacccctccctcaaccggntatcttcttttcatatttctagctacttaaatccaccaagcaaattctggtttcgctatttctggatttaagtacattttctaatgcacccaggccacacgcaggaatggacacagctttcgctctccgcataagttttctagcccagggcaagctctcagaacgtgactcctcgtgcctcgctgcccgcccctcgctccccccactcccgccatcatcccttccgcagagttggctgccaagccgacacccacagattctgcattccacggcttcccagcatcctgctgtctctgcgtggaggaaagggtgctgcgaatcccagtttagataaggaaccccgtcttttcactactacaccgaagcatgcaggaatagggcgttgaccccattctcccagctcagggcacaggtgctaaggccgacccatctcacgggcctgttcaccagtttcccacactcagtacagatcctaacacggtttatgcacttcataaccatgcgtttgtgtttgtcaaactgacaaacgagtgataatgacaaacgaatgacacaagggaaaccaacagccaatcagagcgcaaggttgtgttgccagcatcccagtttcagggacagtcaccgccaagcccgaccctgcaaagggtcgtaaccggacttcggggcaaacttcaggtcccccgcctgctagcccgccaaccccctccgccaggcagccggcgcgcctccaggttccgcccaggcgtcgctgagtggcggcggcggggagaatgcgcgcggcgcagccaatccgggggcgttcctacaccccctgcccgcccccgaccttccagagcagagcaggacactggcgccgcgggtcaggcagctgcgtgcgcgtctcctccaggcagcaaggggaacccgaggccgccggcgcccggaccatgtcgtctccggggccgtcgcagccgccggccgaggacccgccctggcccgcgcgcctcctgcgtgcgcctctggggctgctgcggctggaccccagcgggggcgcgctgctgctatgcggcctcgtagcgctgctgggctggagctggctgcggaggcgccgggcgcggggcatcccgcccgggcccacgccctggcctctggtgggcaacttcggtcacgtgttgctgcctcccttcctccggcggcgaagttggctgagcagcaggaccagggccgcagggattgatccctcggtcataggcccgcaggtgctcctggctcacctagcccgcgtgtacggcagcatcttcagcttctttatcggccactacctggtggtggtcctcagcgacttccacagcgtgcgcgaggcgctggtgcagcaggccgaggtcttcagcgaccgcccgcgggtgccgctcatctccatcgtgaccaaggagaagggtgagcgggaggtcgtgggctgtgggtacgcggatgccgcggatgagtctccaggtgcgtgggggctgcagttcctgtgccccttccggccgcccgcgcccccaggctgcctcacacctgcatcctgaactaacaggtgatggtggtggcggcgcttctcctttctgatgcctttaagatcccatcaagtagtgacggacgcgtgacttgctcatactccaagatcataagtagaaaagtcatccggagattgagggagagtaatagggag

SEQ ID NO: 56 - DNA, 733 bp, Homo sapiens
gggatccggagcccaaatcttctgacaaaactcacacatgcccaccgtgcccagcacctgaattcgagggtgcaccgtcagtcttcctcttccccccaaaacccaaggacaccctcatgatctcccggactcctgaggtcacatgcgtggtggtggacgtaagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaacccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccgcgactctagaggat